SEQUENCE LISTING (141 sequences)

SEQ ID NO: 1 - protein, 39 aa, synthetic construct
  MOD_RES 39: note = C-terminal amidation (-NH2)
HAQGTFTSDFSKYLDGSWAPDFVLWLINGGPSSGAPPPS

SEQ ID NO: 2 - protein, 39 aa, synthetic construct
  MOD_RES 39: note = C-terminal amidation (-NH2)
HAQGTFTSDYSKYLDYMMARDFVQWLIEGGPSSGAPPPS

SEQ ID NO: 3 - protein, 39 aa, synthetic construct
  MOD_RES 39: note = C-terminal amidation (-NH2)
HAQGTFTSDWSKYLDQQMAQDFVDWLINGGPSSGAPPPS

SEQ ID NO: 4 - protein, 39 aa, synthetic construct
  MOD_RES 39: note = C-terminal amidation (-NH2)
HAQGTFTSDHSWYLDKWTAHDFVQWLINGGPSSGAPPPS

SEQ ID NO: 5 - protein, 39 aa, synthetic construct
  MOD_RES 39: note = C-terminal amidation (-NH2)
HAQGTFTSDWSKYLDKPWAGDFVLWLLGGGPSSGAPPPS

SEQ ID NO: 6 - protein, 39 aa, synthetic construct
  MOD_RES 39: note = C-terminal amidation (-NH2)
HAQGTFTSDSSWYLDSYWANDFVNWLMGGGPSSGAPPPS

SEQ ID NO: 7 - protein, 39 aa, synthetic construct
  MOD_RES 39: note = C-terminal amidation (-NH2)
HAQGTFTSDLSKYLDGPWAGDFVWWLIEGGPSSGAPPPS

SEQ ID NO: 8 - protein, 39 aa, synthetic construct
  MOD_RES 39: note = C-terminal amidation (-NH2)
HAQGTFTSDASKYLDRDWTGDFVQWLIDGGPSSGAPPPS

SEQ ID NO: 9 - protein, 39 aa, synthetic construct
  MOD_RES 39: note = C-terminal amidation (-NH2)
HAQGTFTSDESWYLDFQDAPDFVQWLIHGGPSSGAPPPS

SEQ ID NO: 10 - protein, 39 aa, synthetic construct
  MOD_RES 39: note = C-terminal amidation (-NH2)
HAQGTFTSDHSKYLDGAWAADFVMWLLDGGPSSGAPPPS

SEQ ID NO: 11 - protein, 38 aa, synthetic construct
  MOD_RES 38: note = C-terminal amidation (-NH2)
HAQGTFTSDFSKYLDPFYAGLRGLADGGGPSSGAPPPS

SEQ ID NO: 12 - protein, 39 aa, synthetic construct
  MOD_RES 39: note = C-terminal amidation (-NH2)
HAQGTFTSDESWYLDPWDADDFVRWLMQGGPSSGAPPPS

SEQ ID NO: 13 - protein, 38 aa, synthetic construct
  MOD_RES 38: note = C-terminal amidation (-NH2)
HAQGTFTSDDSWYLDPFYAGLRGLADPGGPSSGAPPPS

SEQ ID NO: 14 - protein, 38 aa, synthetic construct
  MOD_RES 38: note = C-terminal amidation (-NH2)
HAQGTFTSDHSKYLDPFYAGLRDLALMGGPSSGAPPPS

SEQ ID NO: 15 - protein, 39 aa, synthetic construct
  MOD_RES 39: note = C-terminal amidation (-NH2)
HAQGTFTSDMSKYLDGPWAGDFVQWLIDGGPSSGAPPPS

SEQ ID NO: 16 - protein, 39 aa, synthetic construct
  MOD_RES 39: note = C-terminal amidation (-NH2)
HAQGTFTSDQSKYLDAPWAGDFVMWLIDGGPSSGAPPPS

SEQ ID NO: 17 - protein, 39 aa, synthetic construct
  MOD_RES 39: note = C-terminal amidation (-NH2)
HAQGTFTSDDSKYLDKPWAPDFVMWLLDGGPSSGAPPPS

SEQ ID NO: 18 - protein, 39 aa, synthetic construct
  MOD_RES 39: note = C-terminal amidation (-NH2)
HAQGTFTSDKSWYLDGPWAGDFVLWLIEGGPSSGAPPPS

SEQ ID NO: 19 - protein, 40 aa, synthetic construct
  VARIANT 2: note = X is 2-aminoisobutyric acid
  MOD_RES 40: note = C-terminal amidation (-NH2)
HXQGTFTSDFSKYLDGSWAPDFVLWLINGGPSSGAPPPSC

SEQ ID NO: 20 - protein, 40 aa, synthetic construct
  VARIANT 2: note = X is 2-aminoisobutyric acid
  MOD_RES 40: note = C-terminal amidation (-NH2)
HXQGTFTSDYSKYLDYMMARDFVQWLIEGGPSSGAPPPSC

SEQ ID NO: 21 - protein, 40 aa, synthetic construct
  VARIANT 2: note = X is 2-aminoisobutyric acid
  MOD_RES 40: note = C-terminal amidation (-NH2)
HXQGTFTSDWSKYLDQQMAQDFVDWLINGGPSSGAPPPSC

SEQ ID NO: 22 - protein, 40 aa, synthetic construct
  VARIANT 2: note = X is 2-aminoisobutyric acid
  MOD_RES 40: note = C-terminal amidation (-NH2)
HXQGTFTSDHSWYLDKWTAHDFVQWLINGGPSSGAPPPSC

SEQ ID NO: 23 - protein, 40 aa, synthetic construct
  VARIANT 2: note = X is 2-aminoisobutyric acid
  MOD_RES 40: note = C-terminal amidation (-NH2)
HXQGTFTSDWSKYLDKPWAGDFVLWLLGGGPSSGAPPPSC

SEQ ID NO: 24 - protein, 40 aa, synthetic construct
  VARIANT 2: note = X is 2-aminoisobutyric acid
  MOD_RES 40: note = C-terminal amidation (-NH2)
HXQGTFTSDSSWYLDSYWANDFVNWLMGGGPSSGAPPPSC

SEQ ID NO: 25 - protein, 39 aa, synthetic construct
  VARIANT 2: note = X is 2-aminoisobutyric acid
  MOD_RES 39: note = C-terminal amidation (-NH2)
HXQGTFTSDLSKYLDGPWAGDFVWWLIEGGPSSGAPPPS

SEQ ID NO: 26 - protein, 40 aa, synthetic construct
  VARIANT 2: note = X is 2-aminoisobutyric acid
  MOD_RES 40: note = C-terminal amidation (-NH2)
HXQGTFTSDASKYLDRDWTGDFVQWLIDGGPSSGAPPPSC

SEQ ID NO: 27 - protein, 40 aa, synthetic construct
  VARIANT 2: note = X is 2-aminoisobutyric acid
  MOD_RES 40: note = C-terminal amidation (-NH2)
HXQGTFTSDESWYLDFQDAPDFVQWLIHGGPSSGAPPPSC

SEQ ID NO: 28 - protein, 40 aa, synthetic construct
  VARIANT 2: note = X is 2-aminoisobutyric acid (Aib)
  MOD_RES 40: note = C-terminal amidation (-NH2)
HXQGTFTSDHSKYLDGAWAADFVMWLLDGGPSSGAPPPSC

SEQ ID NO: 29 - protein, 39 aa, synthetic construct
  VARIANT 2: note = X is 2-aminoisobutyric acid
  MOD_RES 39: note = C-terminal amidation (-NH2)
HXQGTFTSDFSKYLDPFYAGLRGLADGGGPSSGAPPPSC

SEQ ID NO: 30 - protein, 40 aa, synthetic construct
  VARIANT 2: note = X is 2-aminoisobutyric acid
  MOD_RES 40: note = C-terminal amidation (-NH2)
HXQGTFTSDESWYLDPWDADDFVRWLMQGGPSSGAPPPSC

SEQ ID NO: 31 - protein, 39 aa, synthetic construct
  VARIANT 2: note = X is 2-aminoisobutyric acid
  MOD_RES 39: note = C-terminal amidation (-NH2)
HXQGTFTSDDSWYLDPFYAGLRGLADPGGPSSGAPPPSC

SEQ ID NO: 32 - protein, 39 aa, synthetic construct
  VARIANT 2: note = X is 2-aminoisobutyric acid
  MOD_RES 39: note = C-terminal amidation (-NH2)
HXQGTFTSDHSKYLDPFYAGLRDLALMGGPSSGAPPPSC

SEQ ID NO: 33 - protein, 40 aa, synthetic construct
  VARIANT 2: note = X is 2-aminoisobutyric acid
  MOD_RES 40: note = C-terminal amidation (-NH2)
HXQGTFTSDMSKYLDGPWAGDFVQWLIDGGPSSGAPPPSC

SEQ ID NO: 34 - protein, 40 aa, synthetic construct
  VARIANT 2: note = X is 2-aminoisobutyric acid
  MOD_RES 40: note = C-terminal amidation (-NH2)
HXQGTFTSDQSKYLDAPWAGDFVMWLIDGGPSSGAPPPSC

SEQ ID NO: 35 - protein, 40 aa, synthetic construct
  VARIANT 2: note = X is 2-aminoisobutyric acid
  MOD_RES 40: note = C-terminal amidation (-NH2)
HXQGTFTSDDSKYLDKPWAPDFVMWLLDGGPSSGAPPPSC

SEQ ID NO: 36 - protein, 40 aa, synthetic construct
  VARIANT 2: note = X is 2-aminoisobutyric acid
  MOD_RES 40: note = C-terminal amidation (-NH2)
HXQGTFTSDKSWYLDGPWAGDFVLWLIEGGPSSGAPPPSC

SEQ ID NO: 37 - protein, 40 aa, synthetic construct
  VARIANT 2: note = X is 2-aminoisobutyric acid
  MOD_RES 40: note = C-terminal amidation (-NH2)
YXQGTFTSDYSKYLDYMMARDFVQWLIEGGPSSGAPPPSC

SEQ ID NO: 38 - protein, 40 aa, synthetic construct
  VARIANT 2: note = X is 2-aminoisobutyric acid
  MOD_RES 40: note = C-terminal amidation (-NH2)
YXQGTFTSDYSKYLDYMMQRDFVQWLIEGGPSSGAPPPSC

SEQ ID NO: 39 - protein, 40 aa, synthetic construct
  VARIANT 2: note = X is 2-aminoisobutyric acid
  MOD_RES 40: note = C-terminal amidation (-NH2)
YXQGTFTSDYSKLLDYMMQRDFVQWLLEGGPSSGAPPPSC

SEQ ID NO: 40 - protein, 40 aa, synthetic construct
  VARIANT 2: note = X is 2-aminoisobutyric acid
  MOD_RES 40: note = C-terminal amidation (-NH2)
HXQGTFTSDYSKYLDYMMQRDFVQWLIEGGPSSGAPPPSC

SEQ ID NO: 41 - protein, 40 aa, synthetic construct
  VARIANT 2: note = X is 2-aminoisobutyric acid
  MOD_RES 40: note = C-terminal amidation (-NH2)
HXQGTFTSDYSKLLDYMMQRDFVQWLLEGGPSSGAPPPSC

SEQ ID NO: 42 - protein, 40 aa, synthetic construct
  VARIANT 2: note = X is 2-aminoisobutyric acid
  MOD_RES 40: note = C-terminal amidation (-NH2)
HXQGTFTSDKSRYLDYMMARDFVQWLIEGGPSSGAPPPSC

SEQ ID NO: 43 - protein, 40 aa, synthetic construct
  VARIANT 2: note = X is 2-aminoisobutyric acid
  MOD_RES 40: note = C-terminal amidation (-NH2)
YXQGTFTSDWSKYLDQQMAQDFVDWLINGGPSSGAPPPSC

SEQ ID NO: 44 - protein, 40 aa, synthetic construct
  VARIANT 2: note = X is 2-aminoisobutyric acid
  MOD_RES 40: note = C-terminal amidation (-NH2)
YXQGTFTSDWSKYLDQQMQQDFVDWLINGGPSSGAPPPSC

SEQ ID NO: 45 - protein, 40 aa, synthetic construct
  VARIANT 2: note = X is 2-aminoisobutyric acid
  MOD_RES 40: note = C-terminal amidation (-NH2)
YXQGTFTSDWSKYLDQQMQQDFVDWLLNGGPSSGAPPPSC

SEQ ID NO: 46 - protein, 40 aa, synthetic construct
  VARIANT 2: note = X is 2-aminoisobutyric acid
  MOD_RES 40: note = C-terminal amidation (-NH2)
HXQGTFTSDWSKYLDQQMQQDFVDWLINGGPSSGAPPPSC

SEQ ID NO: 47 - protein, 40 aa, synthetic construct
  VARIANT 2: note = X is 2-aminoisobutyric acid
  MOD_RES 40: note = C-terminal amidation (-NH2)
HXQGTFTSDWSKYLDQQMQQDFVDWLLNGGPSSGAPPPSC

SEQ ID NO: 48 - protein, 40 aa, synthetic construct
  VARIANT 2: note = X is 2-aminoisobutyric acid
  MOD_RES 40: note = C-terminal amidation (-NH2)
HXQGTFTSDKSRYLDQQMAQDFVDWLINGGPSSGAPPPSC

SEQ ID NO: 49 - protein, 40 aa, synthetic construct
  VARIANT 2: note = X is 2-aminoisobutyric acid
  MOD_RES 40: note = C-terminal amidation (-NH2)
HXEGTFTSDWSKYLDQQMAQDFVDWLINGGPSSGAPPPSC

SEQ ID NO: 50 - protein, 40 aa, synthetic construct; identical to SEQ ID NO: 37
  VARIANT 2: note = X is 2-aminoisobutyric acid
  MOD_RES 40: note = (8-amino-3,6-dioxaoctanoic acid)2-gamma glutamic acid-C18
  MOD_RES 40: note = C-terminal amidation (-NH2)

SEQ ID NO: 51 - protein, 40 aa, synthetic construct; identical to SEQ ID NO: 38
  VARIANT 2: note = X is 2-aminoisobutyric acid
  MOD_RES 40: note = (8-amino-3,6-dioxaoctanoic acid)2-gamma glutamic acid-C18
  MOD_RES 40: note = C-terminal amidation (-NH2)

SEQ ID NO: 52 - protein, 40 aa, synthetic construct; identical to SEQ ID NO: 39
  VARIANT 2: note = X is 2-aminoisobutyric acid
  MOD_RES 40: note = (8-amino-3,6-dioxaoctanoic acid)2-gamma glutamic acid-C18
  MOD_RES 40: note = C-terminal amidation (-NH2)

SEQ ID NO: 53 - protein, 40 aa, synthetic construct; identical to SEQ ID NO: 40
  VARIANT 2: note = X is 2-aminoisobutyric acid
  MOD_RES 40: note = (8-amino-3,6-dioxaoctanoic acid)2-gamma glutamic acid-C18
  MOD_RES 40: note = C-terminal amidation (-NH2)

SEQ ID NO: 54 - protein, 40 aa, synthetic construct; identical to SEQ ID NO: 41
  VARIANT 2: note = X is 2-aminoisobutyric acid
  MOD_RES 40: note = (8-amino-3,6-dioxaoctanoic acid)2-gamma glutamic acid-C18
  MOD_RES 40: note = C-terminal amidation (-NH2)

SEQ ID NO: 55 - protein, 40 aa, synthetic construct; identical to SEQ ID NO: 42
  VARIANT 2: note = X is 2-aminoisobutyric acid
  MOD_RES 40: note = (8-amino-3,6-dioxaoctanoic acid)2-gamma glutamic acid-C18
  MOD_RES 40: note = C-terminal amidation (-NH2)

SEQ ID NO: 56 - protein, 40 aa, synthetic construct; identical to SEQ ID NO: 43
  VARIANT 2: note = X is 2-aminoisobutyric acid
  MOD_RES 40: note = (8-amino-3,6-dioxaoctanoic acid)2-gamma glutamic acid-C18
  MOD_RES 40: note = C-terminal amidation (-NH2)

SEQ ID NO: 57 - protein, 40 aa, synthetic construct; identical to SEQ ID NO: 44
  VARIANT 2: note = X is 2-aminoisobutyric acid
  MOD_RES 40: note = (8-amino-3,6-dioxaoctanoic acid)2-gamma glutamic acid-C18
  MOD_RES 40: note = C-terminal amidation (-NH2)

SEQ ID NO: 58 - protein, 40 aa, synthetic construct; identical to SEQ ID NO: 45
  VARIANT 2: note = X is 2-aminoisobutyric acid
  MOD_RES 40: note = (8-amino-3,6-dioxaoctanoic acid)2-gamma glutamic acid-C18
  MOD_RES 40: note = C-terminal amidation (-NH2)

SEQ ID NO: 59 - protein, 40 aa, synthetic construct; identical to SEQ ID NO: 46
  VARIANT 2: note = X is 2-aminoisobutyric acid
  MOD_RES 40: note = (8-amino-3,6-dioxaoctanoic acid)2-gamma glutamic acid-C18
  MOD_RES 40: note = C-terminal amidation (-NH2)

SEQ ID NO: 60 - protein, 40 aa, synthetic construct; identical to SEQ ID NO: 47
  VARIANT 2: note = X is 2-aminoisobutyric acid
  MOD_RES 40: note = (8-amino-3,6-dioxaoctanoic acid)2-gamma glutamic acid-C18
  MOD_RES 40: note = C-terminal amidation (-NH2)

SEQ ID NO: 61 - protein, 40 aa, synthetic construct; identical to SEQ ID NO: 48
  VARIANT 2: note = X is 2-aminoisobutyric acid
  MOD_RES 40: note = (8-amino-3,6-dioxaoctanoic acid)2-gamma glutamic acid-C18
  MOD_RES 40: note = C-terminal amidation (-NH2)

SEQ ID NO: 62 - protein, 40 aa, synthetic construct; identical to SEQ ID NO: 49
  VARIANT 2: note = X is 2-aminoisobutyric acid
  MOD_RES 40: note = (8-amino-3,6-dioxaoctanoic acid)2-gamma-glutamic acid-C18
  MOD_RES 40: note = C-terminal amidation (-NH2)

SEQ ID NO: 63 - protein, 39 aa, synthetic construct
  VARIANT 2: note = X is 2-aminoisobutyric acid
  MOD_RES 12: note = (8-amino-3,6-dioxaoctanoic acid)2-gamma-glutamic acid-C18
HXQGTFTSDYSKYLDYMMARDFVQWLIEGGPSSGAPPPS

SEQ ID NO: 64 - protein, 39 aa, synthetic construct
  VARIANT 2: note = X is 2-aminoisobutyric acid
  MOD_RES 12: note = (8-amino-3,6-dioxaoctanoic acid)2-gamma-glutamic acid-C18
YXQGTFTSDYSKYLDYMMARDFVQWLIEGGPSSGAPPPS

SEQ ID NO: 65 - protein, 39 aa, synthetic construct
  VARIANT 2: note = X is 2-aminoisobutyric acid
  MOD_RES 12: note = (8-amino-3,6-dioxaoctanoic acid)2-gamma-glutamic acid-C18
YXQGTFTSDYSKYLDYMMQRDFVQWLIEGGPSSGAPPPS

SEQ ID NO: 66 - protein, 39 aa, synthetic construct
  VARIANT 2: note = X is 2-aminoisobutyric acid
  MOD_RES 12: note = (8-amino-3,6-dioxaoctanoic acid)2-gamma-glutamic acid-C18
  MOD_RES 39: note = C-terminal amidation (-NH2)
YXQGTFTSDYSKYLDYMMQRDFVQWLLEGGPSSGAPPPS

SEQ ID NO: 67 - protein, 39 aa, synthetic construct
  VARIANT 2: note = X is 2-aminoisobutyric acid
  MOD_RES 12: note = (8-amino-3,6-dioxaoctanoic acid)2-gamma-glutamic acid-C18
YXQGTFTSDYSKLLDYMMQRDFVQWLLEGGPSSGAPPPS

SEQ ID NO: 68 - protein, 39 aa, synthetic construct
  VARIANT 2: note = X is 2-aminoisobutyric acid
  MOD_RES 12: note = (8-amino-3,6-dioxaoctanoic acid)2-gamma glutamic acid-C18
HXQGTFTSDYSKYLDYMMQRDFVQWLIEGGPSSGAPPPS

SEQ ID NO: 69 - protein, 39 aa, synthetic construct
  VARIANT 2: note = X is 2-aminoisobutyric acid
  MOD_RES 12: note = (8-amino-3,6-dioxaoctanoic acid)2-gamma-glutamic acid-C18
  MOD_RES 39: note = C-terminal amidation (-NH2)
HXQGTFTSDYSKYLDYMMQRDFVQWLLEGGPSSGAPPPS

SEQ ID NO: 70 - protein, 39 aa, synthetic construct
  VARIANT 2: note = X is 2-aminoisobutyric acid
  MOD_RES 12: note = (8-amino-3,6-dioxaoctanoic acid)2-gamma glutamic acid-C18
HXQGTFTSDYSKLLDYMMQRDFVQWLLEGGPSSGAPPPS

SEQ ID NO: 71 - protein, 39 aa, synthetic construct
  VARIANT 2: note = X is 2-aminoisobutyric acid
  MOD_RES 10: note = (8-amino-3,6-dioxaoctanoic acid)2-gamma glutamic acid-C18
YXQGTFTSDKSKYLDYMMARDFVQWLIEGGPSSGAPPPS

SEQ ID NO: 72 - protein, 39 aa, synthetic construct
  VARIANT 2: note = X is 2-aminoisobutyric acid
  MOD_RES 17: note = (8-amino-3,6-dioxaoctanoic acid)2-gamma glutamic acid-C18
YXQGTFTSDYSKYLDYKMARDFVQWLIEGGPSSGAPPPS

SEQ ID NO: 73 - protein, 39 aa, synthetic construct
  VARIANT 2: note = X is 2-aminoisobutyric acid
  MOD_RES 17: note = (8-amino-3,6-dioxaoctanoic acid)2-gamma glutamic acid-C18
  MOD_RES 39: note = C-terminal amidation (-NH2)
YXQGTFTSDYSKLLDYKMQRDFVQWLLEGGPSSGAPPPS

SEQ ID NO: 74 - protein, 39 aa, synthetic construct; identical to SEQ ID NO: 63
  VARIANT 2: note = X is 2-aminoisobutyric acid
  MOD_RES 12: note = (8-amino-3,6-dioxaoctanoic acid)2-gamma glutamic acid-C18
  MOD_RES 39: note = C-terminal amidation (-NH2)

SEQ ID NO: 75 - protein, 39 aa, synthetic construct; identical to SEQ ID NO: 64
  VARIANT 2: note = X is 2-aminoisobutyric acid
  MOD_RES 12: note = (8-amino-3,6-dioxaoctanoic acid)2-gamma glutamic acid-C18
  MOD_RES 39: note = C-terminal amidation (-NH2)

SEQ ID NO: 76 - protein, 39 aa, synthetic construct; identical to SEQ ID NO: 65
  VARIANT 2: note = X is 2-aminoisobutyric acid
  MOD_RES 12: note = (8-amino-3,6-dioxaoctanoic acid)2-gamma glutamic acid-C18
  MOD_RES 39: note = C-terminal amidation (-NH2)

SEQ ID NO: 77 - protein, 39 aa, synthetic construct; identical to SEQ ID NO: 67
  VARIANT 2: note = X is 2-aminoisobutyric acid
  MOD_RES 12: note = (8-amino-3,6-dioxaoctanoic acid)2-gamma glutamic acid-C18
  MOD_RES 39: note = C-terminal amidation (-NH2)

SEQ ID NO: 78 - protein, 39 aa, synthetic construct; identical to SEQ ID NO: 68
  VARIANT 2: note = X is 2-aminoisobutyric acid
  MOD_RES 12: note = (8-amino-3,6-dioxaoctanoic acid)2-gamma glutamic acid-C18
  MOD_RES 39: note = C-terminal amidation (-NH2)

SEQ ID NO: 79 - protein, 39 aa, synthetic construct; identical to SEQ ID NO: 70
  VARIANT 2: note = X is 2-aminoisobutyric acid
  MOD_RES 12: note = (8-amino-3,6-dioxaoctanoic acid)2-gamma glutamic acid-C18
  MOD_RES 39: note = C-terminal amidation (-NH2)

SEQ ID NO: 80 - protein, 39 aa, synthetic construct; identical to SEQ ID NO: 71
  VARIANT 2: note = X is 2-aminoisobutyric acid
  MOD_RES 10: note = (8-amino-3,6-dioxaoctanoic acid)2-gamma glutamic acid-C18
  MOD_RES 39: note = C-terminal amidation (-NH2)

SEQ ID NO: 81 - protein, 39 aa, synthetic construct; identical to SEQ ID NO: 72
  VARIANT 2: note = X is 2-aminoisobutyric acid
  MOD_RES 17: note = (8-amino-3,6-dioxaoctanoic acid)2-gamma glutamic acid-C18
  MOD_RES 39: note = C-terminal amidation (-NH2)

SEQ ID NO: 82 - protein, 40 aa, synthetic construct; identical to SEQ ID NO: 20
  VARIANT 2: note = X is 2-aminoisobutyric acid
  MOD_RES 12: note = N-Biotinoyl-N prime-(6-maleimidohexanoyl)hydrazide
  MOD_RES 40: note = (8-amino-3,6-dioxaoctanoic acid)2-gamma glutamic acid-C18
  MOD_RES 40: note = C-terminal amidation (-NH2)

SEQ ID NO: 83 - protein, 40 aa, synthetic construct; identical to SEQ ID NO: 20
  VARIANT 2: note = X is 2-aminoisobutyric acid
  MOD_RES 12: note = (8-amino-3,6-dioxaoctanoic acid)2-gamma glutamic acid-C18
  MOD_RES 40: note = N-Biotinoyl-N prime-(6-maleimidohexanoyl)hydrazide
  MOD_RES 40: note = C-terminal amidation (-NH2)

SEQ ID NO: 84 - protein, 39 aa, synthetic construct
  VARIANT 2: note = X is 2-aminoisobutyric acid
  VARIANT 20: note = X is 2-aminoisobutyric acid
  MOD_RES 12: note = (8-amino-3,6-dioxaoctanoic acid)2-gamma glutamic acid-C18
  MOD_RES 39: note = C-terminal amidation (-NH2)
YXQGTFTSDYSKLLDYMMQXDFVQWLLEGGPSSGAPPPS

SEQ ID NO: 85 - protein, 39 aa, synthetic construct
  VARIANT 2: note = X is 2-aminoisobutyric acid
  VARIANT 20: note = X is 2-aminoisobutyric acid
  MOD_RES 12: note = (8-amino-3,6-dioxaoctanoic acid)2-gamma glutamic acid-C18
  MOD_RES 39: note = C-terminal amidation (-NH2)
YXQGTFTSDYSKLLDYMMQXDFVQYLLEGGPSSGAPPPS

SEQ ID NO: 86 - protein, 39 aa, synthetic construct
  VARIANT 2: note = X is 2-aminoisobutyric acid
  MOD_RES 16: note = N-Biotinoyl-Nprime-(6-maleimidohexanoyl)hydrazide
  MOD_RES 12: note = (8-amino-3,6-dioxaoctanoic acid)2-gamma glutamic acid-C18
  MOD_RES 39: note = C-terminal amidation (-NH2)
YXQGTFTSDYSKLLDKMMQRDFVQWLLEGGPSSGAPPPS

SEQ ID NO: 87 - protein, 39 aa, synthetic construct; identical to SEQ ID NO: 73
  VARIANT 2: note = X is 2-aminoisobutyric acid
  MOD_RES 12: note = (8-amino-3,6-dioxaoctanoic acid)2-gamma glutamic acid-C18
  MOD_RES 17: note = N-Biotinoyl-Nprime-(6-maleimidohexanoyl)hydrazide
  MOD_RES 39: note = C-terminal amidation (-NH2)

SEQ ID NO: 88 - protein, 39 aa, synthetic construct
  VARIANT 2: note = X is 2-aminoisobutyric acid
  MOD_RES 12: note = (8-amino-3,6-dioxaoctanoic acid)2-gamma glutamic acid-C18
  MOD_RES 20: note = N-Biotinoyl-Nprime-(6-maleimidohexanoyl)hydrazide
  MOD_RES 39: note = C-terminal amidation (-NH2)
YXQGTFTSDYSKLLDYMMQKDFVQWLLEGGPSSGAPPPS

SEQ ID NO: 89 - protein, 39 aa, synthetic construct
  VARIANT 2: note = X is 2-aminoisobutyric acid
  MOD_RES 24: note = N-Biotinoyl-Nprime-(6-maleimidohexanoyl)hydrazide
  MOD_RES 12: note = (8-amino-3,6-dioxaoctanoic acid)2-gamma glutamic acid-C18
  MOD_RES 39: note = C-terminal amidation (-NH2)
YXQGTFTSDYSKLLDYMMQRDFVKWLLEGGPSSGAPPPS

SEQ ID NO: 90 - protein, 40 aa, synthetic construct
  VARIANT 2: note = X is 2-aminoisobutyric acid
  MOD_RES 12: note = (8-amino-3,6-dioxaoctanoic acid)2-gamma glutamic acid-C18
  MOD_RES 40: note = N-Biotinoyl-Nprime-(6-maleimidohexanoyl)hydrazide
  MOD_RES 40: note = C-terminal amidation (-NH2)
YXQGTFTSDYSKLLDYMMQRDFVQWLLEGGPSSGAPPPSK

SEQ ID NO: 91 - protein, 39 aa, synthetic construct
  VARIANT 2: note = X is 2-aminoisobutyric acid
  VARIANT 20: note = X is 2-aminoisobutyric acid
  MOD_RES 12: note = (8-amino-3,6-dioxaoctanoic acid)2-gamma glutamic acid-C18
  MOD_RES 17: note = N-Biotinoyl-Nprime-(6-maleimidohexanoyl)hydrazide
  MOD_RES 39: note = C-terminal amidation (-NH2)
YXQGTFTSDYSKLLDYKMQXDFVQWLLEGGPSSGAPPPS

SEQ ID NO: 92 - protein, 40 aa, synthetic construct
  VARIANT 2: note = X is 2-aminoisobutyric acid
  VARIANT 20: note = X is 2-aminoisobutyric acid
  MOD_RES 12: note = (8-amino-3,6-dioxaoctanoic acid)2-gamma glutamic acid-C18
  MOD_RES 40: note = N-Biotinoyl-Nprime-(6-maleimidohexanoyl)hydrazide
  MOD_RES 40: note = C-terminal amidation (-NH2)
YXQGTFTSDYSKLLDYMMQXDFVQYLLEGGPSSGAPPPSK

SEQ ID NO: 93 - protein, 39 aa, synthetic construct
  VARIANT 2: note = X is 2-aminoisobutyric acid
  MOD_RES 12: note = (8-amino-3,6-dioxaoctanoic acid)2-gamma glutamic acid-C18
  MOD_RES 39: note = C-terminal amidation (-NH2)
YXQGTFTSDYSKLLDYYAQRDFVQWLLEGGPSSGAPPPS

SEQ ID NO: 94 - protein, 39 aa, synthetic construct
  VARIANT 2: note = X is 2-aminoisobutyric acid
  MOD_RES 12: note = (8-amino-3,6-dioxaoctanoic acid)2-gamma glutamic acid-C18
  MOD_RES 39: note = C-terminal amidation (-NH2)
  MOD_RES 39: note = C-terminal amidation (-NH2)
YXQGTFTSDYSKLLDYYIQRDFVQWLLEGGPSSGAPPPS

SEQ ID NO: 95 - protein, 39 aa, synthetic construct
  VARIANT 2: note = X is 2-aminoisobutyric acid
  MOD_RES 12: note = (8-amino-3,6-dioxaoctanoic acid)2-gamma glutamic acid-C18
  MOD_RES 39: note = C-terminal amidation (-NH2)
YXQGTFTSDYSKLLDQQAQRDFVQWLLEGGPSSGAPPPS

SEQ ID NO: 96 - protein, 39 aa, synthetic construct
  VARIANT 2: note = X is 2-aminoisobutyric acid
  VARIANT 17: note = X is methoxinine
  VARIANT 18: note = X is Methoxinine
  MOD_RES 12: note = (8-amino-3,6-dioxaoctanoic acid)2-gamma glutamic acid-C18
  MOD_RES 39: note = C-terminal amidation (-NH2)
YXQGTFTSDYSKLLDYXXQRDFVQWLLEGGPSSGAPPPS

SEQ ID NO: 97 - protein, 39 aa, synthetic construct
  VARIANT 2: note = X is 2-aminoisobutyric acid
  MOD_RES 12: note = (8-amino-3,6-dioxaoctanoic acid)2-gamma glutamic acid-C18
  MOD_RES 24: note = N-Biotinoyl-Nprime-(6-maleimidohexanoyl)hydrazide
  MOD_RES 39: note = C-terminal amidation (-NH2)
YXQGTFTSDYSKYLDYMMQRDFVKWLLEGGPSSGAPPPS

SEQ ID NO: 98 - protein, 40 aa, synthetic construct
  VARIANT 2: note = X is 2-aminoisobutyric acid
  MOD_RES 12: note = (8-amino-3,6-dioxaoctanoic acid)2-gamma glutamic acid-C18
  MOD_RES 40: note = N-Biotinoyl-Nprime-(6-maleimidohexanoyl)hydrazide
  MOD_RES 40: note = C-terminal amidation (-NH2)
YXQGTFTSDYSKYLDYMMQRDFVQWLLEGGPSSGAPPPSK

SEQ ID NO: 99 - protein, 39 aa, synthetic construct
  VARIANT 2: note = X is 2-aminoisobutyric acid
  MOD_RES 12: note = (8-amino-3,6-dioxaoctanoic acid)2-gamma glutamic acid-C18
  MOD_RES 39: note = C-terminal amidation (-NH2)
YXQGTFTSDYSKLLDYMMQHDFVQWLLEGGPSSGAPPPS

SEQ ID NO: 100 - protein, 39 aa, synthetic construct
  VARIANT 2: note = X is 2-aminoisobutyric acid
  MOD_RES 12: note = (8-amino-3,6-dioxaoctanoic acid)2-gamma glutamic acid-C18
  MOD_RES 39: note = C-terminal amidation (-NH2)
YXQGTFTSDYSKLLDYMMQQDFVQWLLEGGPSSGAPPPS

SEQ ID NO: 101 - protein, 39 aa, synthetic construct; identical to SEQ ID NO: 67
  VARIANT 2: note = X is 2-aminoisobutyric acid
  MOD_RES 12: note = (8-amino-3,6-dioxaoctanoic acid)2-gamma glutamic acid-C18
  MOD_RES 20: note = alpha-methyl-arginine
  MOD_RES 39: note = C-terminal amidation (-NH2)

SEQ ID NO: 102 - protein, 39 aa, synthetic construct; identical to SEQ ID NO: 67
  VARIANT 2: note = X is 2-aminoisobutyric acid
  MOD_RES 12: note = (8-amino-3,6-dioxaoctanoic acid)-gamma glutamic acid-C20
  MOD_RES 39: note = C-terminal amidation (-NH2)

SEQ ID NO: 103 - protein, 39 aa, synthetic construct; identical to SEQ ID NO: 67
  VARIANT 2: note = X is 2-aminoisobutyric acid
  MOD_RES 12: note = (8-amino-3,6-dioxaoctanoic acid)2-gamma glutamic acid-C20
  MOD_RES 39: note = C-terminal amidation (-NH2)

SEQ ID NO: 104 - protein, 39 aa, synthetic construct
  VARIANT 2: note = X is 2-aminoisobutyric acid
  MOD_RES 12: note = (8-amino-3,6-dioxaoctanoic acid)2-gamma glutamic acid-C20
  MOD_RES 39: note = C-terminal amidation (-NH2)
YXQGTFTSDYSKLLDYMMQRDFVBWLLEGGPSSGAPPPS

SEQ ID NO: 105 - protein, 40 aa, synthetic construct; identical to SEQ ID NO: 90
  VARIANT 2: note = X is 2-aminoisobutyric acid
  MOD_RES 12: note = (8-amino-3,6-dioxaoctanoic acid)2-gamma glutamic acid-C20
  MOD_RES 40: note = N-Biotinoyl-Nprime-(6-maleimidohexanoyl)hydrazide
  MOD_RES 40: note = C-terminal amidation (-NH2)

SEQ ID NO: 106 - protein, 39 aa, synthetic construct; identical to SEQ ID NO: 66
  VARIANT 2: note = X is 2-aminoisobutyric acid
  MOD_RES 12: note = (8-amino-3,6-dioxaoctanoic acid)2-gamma glutamic acid-C18
  MOD_RES 20: note = alpha-methyl-arginine
  MOD_RES 39: note = C-terminal amidation (-NH2)

SEQ ID NO: 107 - protein, 39 aa, synthetic construct; identical to SEQ ID NO: 66
  VARIANT 2: note = X is 2-aminoisobutyric acid
  MOD_RES 12: note = (8-amino-3,6-dioxaoctanoic acid)2-gamma glutamic acid-C20
  MOD_RES 20: note = alpha-methyl arginine
  MOD_RES 39: note = C-terminal amidation (-NH2)

SEQ ID NO: 108 - protein, 39 aa, synthetic construct; identical to SEQ ID NO: 67
  VARIANT 2: note = X is 2-aminoisobutyric acid
  MOD_RES 12: note = (8-amino-3,6-dioxaoctanoic acid)2-gamma glutamic acid-C20
  MOD_RES 20: note = alpha-methyl arginine
  MOD_RES 39: note = C-terminal amidation (-NH2)

SEQ ID NO: 109 - protein, 39 aa, synthetic construct; identical to SEQ ID NO: 97
  MOD_RES 12: note = (8-amino-3,6-dioxaoctanoic acid)2-gamma glutamic acid-C18
  VARIANT 2: note = X is 2-aminoisobutyric acid
  MOD_RES 20: note = alpha-methyl-arginine
  MOD_RES 24: note = N-Biotinoyl-Nprime-(6-maleimidohexanoyl)hydrazide
  MOD_RES 39: note = C-terminal amidation (-NH2)

SEQ ID NO: 110 - protein, 39 aa, synthetic construct; identical to SEQ ID NO: 97
  VARIANT 2: note = X is 2-aminoisobutyric acid
  MOD_RES 12: note = (8-amino-3,6-dioxaoctanoic acid)2-gamma glutamic acid-C20
  MOD_RES 20: note = alpha-methyl-arginine
  MOD_RES 24: note = N-Biotinoyl-Nprime-(6-maleimidohexanoyl)hydrazide
  MOD_RES 39: note = C-terminal amidation (-NH2)

SEQ ID NO: 111 - protein, 39 aa, synthetic construct; identical to SEQ ID NO: 89
  VARIANT 2: note = X is 2-aminoisobutyric acid
  MOD_RES 12: note = (8-amino-3,6-dioxaoctanoic acid)2-gamma glutamic acid-C18
  MOD_RES 20: note = alpha-methyl-arginine
  MOD_RES 24: note = N-Biotinoyl-Nprime-(6-maleimidohexanoyl)hydrazide
  MOD_RES 39: note = C-terminal amidation (-NH2)

SEQ ID NO: 112 - protein, 39 aa, synthetic construct
  VARIANT 2: note = X is 2-aminoisobutyric acid
  MOD_RES 12: note = (8-amino-3,6-dioxaoctanoic acid)2-gamma glutamic acid-C20
  MOD_RES 20: note = alpha-methyl-arginine
  MOD_RES 24: note = N-Biotinoyl-Nprime-(6-maleimidohexanoyl)hydrazide
  MOD_RES 39: note = C-terminal amidation (-NH2)
YXQGTFTSDYSKLLDYMMQRDFVRWLLEGGPSSGAPPPS

SEQ ID NO: 113 - protein, 40 aa, synthetic construct; identical to SEQ ID NO: 19
  VARIANT 2: note = X is 2-aminoisobutyric acid
  MOD_RES 40: note = (8-amino-3,6-dioxaoctanoic acid)2-gamma glutamic acid-C18
  MOD_RES 40: note = C-terminal amidation (-NH2)

SEQ ID NO: 114 - protein, 40 aa, synthetic construct; identical to SEQ ID NO: 20
  VARIANT 2: note = X is 2-aminoisobutyric acid
  MOD_RES 40: note = (8-amino-3,6-dioxaoctanoic acid)2-gamma glutamic acid-C18
  MOD_RES 40: note = C-terminal amidation (-NH2)

SEQ ID NO: 115 - protein, 40 aa, synthetic construct; identical to SEQ ID NO: 21
  VARIANT 2: note = X is 2-aminoisobutyric acid
  MOD_RES 40: note = (8-amino-3,6-dioxaoctanoic acid)2-gamma glutamic acid-C18
  MOD_RES 40: note = C-terminal amidation (-NH2)

SEQ ID NO: 116 - protein, 40 aa, synthetic construct; identical to SEQ ID NO: 22
  VARIANT 2: note = X is 2-aminoisobutyric acid
  MOD_RES 40: note = (8-amino-3,6-dioxaoctanoic acid)2-gamma glutamic acid-C18
  MOD_RES 40: note = C-terminal amidation (-NH2)

SEQ ID NO: 117 - protein, 40 aa, synthetic construct; identical to SEQ ID NO: 23
  VARIANT 2: note = X is 2-aminoisobutyric acid
  MOD_RES 40: note = (8-amino-3,6-dioxaoctanoic acid)2-gamma glutamic acid-C18
  MOD_RES 40: note = C-terminal amidation (-NH2)

SEQ ID NO: 118 - protein, 40 aa, synthetic construct; identical to SEQ ID NO: 24
  VARIANT 2: note = X is 2-aminoisobutyric acid
  MOD_RES 40: note = (8-amino-3,6-dioxaoctanoic acid)2-gamma glutamic acid-C18
  MOD_RES 40: note = C-terminal amidation (-NH2)

SEQ ID NO: 119 - protein, 40 aa, synthetic construct
  VARIANT 2: note = X is 2-aminoisobutyric acid
  MOD_RES 40: note = (8-amino-3,6-dioxaoctanoic acid)2-gamma glutamic acid-C18
  MOD_RES 40: note = C-terminal amidation (-NH2)
HXQGTFTSDLSKYLDGPWAGDFVWWLIEGGPSSGAPPPSC

SEQ ID NO: 120 - protein, 40 aa, synthetic construct; identical to SEQ ID NO: 26
  VARIANT 2: note = X is 2-aminoisobutyric acid
  MOD_RES 40: note = (8-amino-3,6-dioxaoctanoic acid)2-gamma glutamic acid-C18
  MOD_RES 40: note = C-terminal amidation (-NH2)

SEQ ID NO: 121 - protein, 40 aa, synthetic construct; identical to SEQ ID NO: 27
  VARIANT 2: note = X is 2-aminoisobutyric acid
  MOD_RES 40: note = (8-amino-3,6-dioxaoctanoic acid)2-gamma glutamic acid-C18
  MOD_RES 40: note = C-terminal amidation (-NH2)

SEQ ID NO: 122 - protein, 40 aa, synthetic construct; identical to SEQ ID NO: 28
  VARIANT 2: note = X is 2-aminoisobutyric acid
  MOD_RES 40: note = (8-amino-3,6-dioxaoctanoic acid)2-gamma glutamic acid-C18
  MOD_RES 40: note = C-terminal amidation (-NH2)

SEQ ID NO: 123 - protein, 39 aa, synthetic construct; identical to SEQ ID NO: 29
  VARIANT 2: note = X is 2-aminoisobutyric acid
  MOD_RES 39: note = (8-amino-3,6-dioxaoctanoic acid)2-gamma glutamic acid-C18
  MOD_RES 39: note = C-terminal amidation (-NH2)

SEQ ID NO: 124 - protein, 40 aa, synthetic construct; identical to SEQ ID NO: 30
  VARIANT 2: note = X is 2-aminoisobutyric acid
  MOD_RES 40: note = (8-amino-3,6-dioxaoctanoic acid)2-gamma glutamic acid-C18
  MOD_RES 40: note = C-terminal amidation (-NH2)

SEQ ID NO: 125 - protein, 39 aa, synthetic construct; identical to SEQ ID NO: 31
  VARIANT 2: note = X is 2-aminoisobutyric acid
  MOD_RES 39: note = (8-amino-3,6-dioxaoctanoic acid)2-gamma glutamic acid-C18
  MOD_RES 39: note = C-terminal amidation (-NH2)

SEQ ID NO: 126 - protein, 39 aa, synthetic construct; identical to SEQ ID NO: 32
  MOD_RES 39: note = (8-amino-3,6-dioxaoctanoic acid)2-gamma glutamic acid-C18
  VARIANT 2: note = X is 2-aminoisobutyric acid
  MOD_RES 39: note = C-terminal amidation (-NH2)

SEQ ID NO: 127 - protein, 40 aa, synthetic construct; identical to SEQ ID NO: 33
  VARIANT 2: note = X is 2-aminoisobutyric acid
  MOD_RES 40: note = (8-amino-3,6-dioxaoctanoic acid)2-gamma glutamic acid-C18
  MOD_RES 40: note = C-terminal amidation (-NH2)

SEQ ID NO: 128 - protein, 40 aa, synthetic construct; identical to SEQ ID NO: 34
  VARIANT 2: note = X is 2-aminoisobutyric acid
  MOD_RES 40: note = (8-amino-3,6-dioxaoctanoic acid)2-gamma glutamic acid-C18
  MOD_RES 40: note = C-terminal amidation (-NH2)

SEQ ID NO: 129 - protein, 40 aa, synthetic construct; identical to SEQ ID NO: 35
  VARIANT 2: note = X is 2-aminoisobutyric acid
  MOD_RES 40: note = (8-amino-3,6-dioxaoctanoic acid)2-gamma glutamic acid-C18
  MOD_RES 40: note = C-terminal amidation (-NH2)

SEQ ID NO: 130 - protein, 40 aa, synthetic construct; identical to SEQ ID NO: 36
  VARIANT 2: note = X is 2-aminoisobutyric acid
  MOD_RES 40: note = (8-amino-3,6-dioxaoctanoic acid)2-gamma glutamic acid-C18
  MOD_RES 40: note = C-terminal amidation (-NH2)

SEQ ID NO: 131 - protein, 40 aa, synthetic construct
  VARIANT 1: note = X is H or Y
  VARIANT 2: note = X is A or 2-aminoisobutyric acid
  VARIANT 10: note = X is A or R or N or D or Q or E or G or H or I or L or K or M or F or P or S or T or W or Y or V
  VARIANT 12: note = X is R or W or K
  VARIANT 13: note = X is L or Y
  VARIANT 16: note = X is A or R or N or D or Q or E or G or H or I or L or K or M or F or P or S or T or W or Y or V
  VARIANT 17: note = X is A or R or N or D or Q or E or G or H or I or L or K or M or F or P or S or T or W or Y or V or methoxinine or Aib or alpha-methyl-arginine
  VARIANT 18: note = X is A or R or N or D or Q or E or G or H or I or L or K or M or F or P or S or T or W or Y or V or methoxinine or Aib or alpha-methyl-arginine
  VARIANT 19: note = X is Q or A or T
  VARIANT 20: note = X is A or R or N or D or Q or E or G or H or I or L or K or M or F or P or S or T or W or Y or V or methoxinine or Aib or alpha-methyl-arginine
  VARIANT 21: note = X is D or L
  VARIANT 22: note = X is F or R
  VARIANT 23: note = X is V or G or D
  VARIANT 24: note = X is A or R or N or D or Q or E or G or H or I or L or K or M or F or P or S or T or W or Y or V
  VARIANT 25: note = X is W or Y or A
  VARIANT 26: note = X is L or D
  VARIANT 27: note = X is I or L or M or G or P
  VARIANT 28: note = X is A or R or N or D or Q or E or G or H or I or L or K or M or F or P or S or T or W or Y or V
  VARIANT 30: note = X is G or P
  VARIANT 31: note = X is P or S
  VARIANT 33: note = X is S or G
  VARIANT 34: note = X is G or A
  VARIANT 38: note = X is P or S
  VARIANT 35: note = X is A or P
  VARIANT 39: note = X is S or C or absent
  VARIANT 40: note = X is C or K or absent
XXZGTFTSDXSXXLDXXXXXXXXXXXXXGXXSXXXPPXXX

SEQ ID NO: 132 - protein, 30 aa, synthetic construct
  VARIANT 2: note = X is 2-aminoisobutyric acid
HXQGTFTSDYSKYLDEQAAKEFVQWLMNTC

SEQ ID NO: 133 - protein, 39 aa, synthetic construct
  VARIANT 2: note = X is 2-aminoisobutyric acid
  VARIANT 20: note = X is 2-aminoisobutyric acid
  VARIANT 29: note = X is 2-aminoisobutyric acid
  VARIANT 34: note = X is 2-aminoisobutyric acid
  MOD_RES 13: note = alpha-methyl-leucine
  MOD_RES 17: note = ((2-[2-(2-amino-ethoxy)-ethoxy]-acetyl)-(gammaGlu)- CO-(CH2)18-CO2H)
  MOD_RES 39: note = C-terminal amidation (-NH2)
YXQGTFTSDYSILLDKKAQXAFIEYLLEXGPSSXAPPPS

SEQ ID NO: 134 - protein, 40 aa, synthetic construct; identical to SEQ ID NO: 90
  VARIANT 2: note = X is A or R or N or D or C or Q or E or G or H or I or L or K or M or F or P or S or T or W or Y or V

SEQ ID NO: 135 - protein, 40 aa, synthetic construct; identical to SEQ ID NO: 90
  VARIANT 2: note = X is 2-aminoisobutyric acid

SEQ ID NO: 136 - protein, 39 aa, synthetic construct
  VARIANT 2: note = X is 2-aminoisobutyric acid
  VARIANT 13: note = X is 2-aminoisobutyric acid
  MOD_RES 20: note = (8-amino-3,6-dioxaoctanoic acid)2-gamma glutamic acid-C20
YXEGTFTSDYSIXLDKIAQKAFVQWLIAGGPSSGAPPPS

SEQ ID NO: 137 - protein, 31 aa, synthetic construct
HAEGTFTSDVSSYLEGQAAKEFIAWLVKGRG

SEQ ID NO: 138 - protein, 30 aa, synthetic construct
  VARIANT 30: note = X is O or H
HSQGTFTSDYSKYLDSRRAQDFVQWLMNTX

SEQ ID NO: 139 - protein, 42 aa, synthetic construct
YAEGTFISDYSIAMDKIHQQDFVNWLLAQKGKKNDWKHNITQ

SEQ ID NO: 140 - protein, 39 aa, synthetic construct
  VARIANT 10: note = X is any amino acid
  VARIANT 12: note = Xis K or W
  VARIANT 16: note = X is any amino acid
  VARIANT 17: note = X is any amino acid
  VARIANT 18: note = X is any amino acid
  VARIANT 20: note = X is any amino acid
  VARIANT 24: note = X is any amino acid
  VARIANT 27: note = X is L or M or I
  VARIANT 28: note = X is any amino acid
HAQGTFTSDXSXYLDXXXAXDFVXWLXXGGPSSGAPPPS

SEQ ID NO: 141 - protein, 40 aa, synthetic construct
  VARIANT 1: note = X is H or Y
  VARIANT 2: note = X is A or Aib
  VARIANT 10: note = Xis Y or K or W
  VARIANT 13: note = X is L or Y
  VARIANT 16: note = X is Y or Q or K
  VARIANT 17: note = X is M or Y or Q or K
  VARIANT 18: note = X is A or I or M
  VARIANT 19: note = X is A or Q
  VARIANT 20: note = X is R or Q or Aib or K
  VARIANT 24: note = X is Q or D or K
  VARIANT 27: note = X is I or L
  VARIANT 28: note = X is N or E
  VARIANT 40: note = X is K or absent
XXQGTFTSDXSKXLDXXXXXDFVXWLXXGGPSSGAPPPSX

(19) United States
(12) Patent Application Publication
(10) Pub. No.: US 2025/0296960 A1
(43) Pub. Date: Sep. 25, 2025

(54) PEPTIDE COMPOSITIONS AND METHODS OF USE THEREOF

(71) Applicant: D&D Pharmatech Inc., Gyeonggi-do (KR)

(72) Inventors: Eun Ji PARK, Seoul (KR); Hyegyeong YANG, Gyeonggi-do (KR); Kyungsub SHIN, Gyeonggi-do (KR)

(21) Appl. No.: 18/869,214

(22) PCT Filed: May 26, 2023
(86) PCT No.: PCT/IB2023/055433

§ 371 Date: Nov. 25, 2024

Related U.S. Application Data
(60) Provisional application No. 63/346,605, filed on May 27, 2022.

(51) Int. Cl. C07K 14/00; A61K 38/00; A61P 3/04

(52) U.S. Cl. CPC C07K 14/001; A61P 3/04; A61K 38/00

(57) ABSTRACT

Triple agonist peptides having activities at each of GLP-1, Glucagon and GIP receptors are provided. Triple agonist analogs having one or more biotin moieties and/or fatty acid moieties conjugated thereto for improved bioavailability and pharmacokinetics are also described. Compositions and formulations of these triple agonist peptides are particularly suited for treating, alleviating, and/or preventing one or more metabolic diseases such as obesity, diabetes mellitus, or non-alcoholic fatty liver disease. Compositions and methods of use thereof are also described for treating, alleviating, and/or preventing one or more neurodegenerative disease such as Alzheimer's disease (AD) and Parkinson's disease (PD).

### CROSS-REFERENCE TO RELATED APPLICATION

This application claims the benefit of and priority to U.S. Ser. No. 63/346,605 filed May 27, 2022, and which is incorporated by referenced herein in its entirety.

### REFERENCE TO SEQUENCE LISTING

The Sequence Listing submitted as a text file named “DDP_107_PCT_ST26.xml” created on Mar. 25, 2023, and having a size of 287,384 bytes is hereby incorporated by reference pursuant to 37 C.F.R. § 1.834(c)(1).

### FIELD OF THE INVENTION

The invention is generally in the field of peptides and analogs thereof having activities to all of glucagon, GLP-1, and GIP receptors and uses thereof.

### BACKGROUND OF THE INVENTION

Recently, with economic development and medical advancement, aging populations are rapidly growing. Aging increases the risk of chronic diseases such as dementias, heart disease, type 2 diabetes, arthritis, and cancer. Mortality rates of heart and cerebrovascular diseases, which are complications accompanying obesity, are ranked as first and second. Obesity is suggested as a cause of various adult diseases such as diabetes mellitus, and non-alcoholic fatty liver disease.

Obesity refers to a state in which fat is accumulated at higher amounts than normal, and the most accurate method for evaluation of obesity is to measure body fat mass. However, accurate measurement of fat mass is costly, and thus it is evaluated using indirect methods. The most commonly used indirect methods are to measure body mass index (BMI) and waist circumference. The World Health Organization (WHO) announced classifications based on data relating BMI to mortality risk, which are based on normal weight: 18.5 to 24.9 kg/m², overweight: 25 to 29.9 kg/m², and obese: 30 kg/m² or more.

The causes of obesity are known as energy imbalances due to excessive calorie intake and relatively decreased activities, and the resulting increase in body fat. However, it is difficult to assign only one factor because various risk factors such as eating habit, lifestyle, age, race, genetic factors, etc. are involved in obesity. Obese patients are primarily advised to control their weight through healthier diet and physical activity, but when these methods are not effective, patients can be treated with medication or surgery. These often provide limited efficacy.

Diabetes is classified into insulin-dependent diabetes (type I diabetes), insulin-independent diabetes (type II diabetes), and malnutrition-related diabetes mellitus (MRDM). The type II diabetes which accounts for more than 90% of diabetic patients are metabolic diseases characterized by hyperglycemia and are reported to be caused by decreased insulin secretion of pancreatic beta cells or increased insulin resistance in peripheral tissues due to genetic, metabolic, and environmental factors. In this regard, when body fat increases, the insulin sensitivity is decreased. Accumulation of abdominal fat is known especially to be related to glucose intolerance. Also, it is known that insulin resistance is closely correlated with obesity in patients suffering from type II diabetes, with the more severe the obesity, the greater the insulin resistance.

Non-alcoholic fatty liver diseases (NAFLD) refer to a series of diseases including simple steatosis with excessive accumulation of fat in the liver cells independent of alcohol consumption, non-alcoholic steatohepatitis (NASH) including hepatocellular injury (hepatocellular ballooning), inflammation, fibrosis, and, in more advanced cases, cirrhosis. The prevalence rate of non-alcoholic fatty liver disease is rapidly increasing with the increase in the prevalence rate of obesity all over the world, and although the prevalence rate of diabetes varies from country to country, it accounts for about 20 to 30% of the total populations in Western countries, and the incidence rate thereof reaches about 16% in Korea.

GLP-1 is a hormone secreted by the small intestine stimulated by food intake. GLP-1 promotes insulin secretion in the pancreas in a blood glucose-dependent manner and inhibits the secretion of glucagon, thus helping the action of lowering blood glucose levels. Additionally, GLP-1 slows digestive action in the gastrointestinal tract by acting as a satiety factor and reduces the amount of food intake by delaying the time for emptying digested food in the gastrointestinal tract. Administration of GLP-1 to rats was reported to have effects of inhibiting food intake and reducing body weight, and these effects were confirmed to occur equally both in normal and obese states, thus showing the potential of GLP-1 as an agent for treating obesity.

GIP, one of the gastrointestinal hormones secreted by the stimulation of food intake, as is the case of GLP-1, is a hormone consisting of 42 amino acids secreted by the intestinal K-cells. GIP was reported to perform the functions of promoting the secretion of insulin in the pancreas in a blood glucose-dependent manner and helping to lower the blood glucose levels, thereby exhibiting the effects of increasing the activation of GLP-1, anti-inflammation, etc.

Glucagon is produced in the pancreas when the blood glucose levels fall due to reasons such as medications, diseases, deficiency in hormones or enzymes, etc. Glucagon sends a signal for glycogen breakdown in the liver to induce the release of glucose and increases blood glucose levels to a normal level. In addition to the effect of increasing the blood glucose levels, glucagon suppresses appetite in animals and humans and activates hormone-sensitive lipase of adipocytes to promote lipolysis and energy expenditure, thereby showing an anti-obesity effect.

GLP-1 is being developed as a therapeutic agent for treating diabetes and obesity, based on the effects of GLP-1 controlling blood glucose levels and reducing body weight. Exendin-4, prepared from lizard venom and having an amino acid homology of about 50% with GLP-1, is under development as a therapeutic agent for treating the same kinds of diseases. However, the therapeutic agents containing GLP-1 and exendin-4 were reported to show side-effects such as vomiting and nausea (Syed Y Y., Drugs, 2015 July; 75 (10): 1141-52).

For maximization of body weight reduction and as an alternative to GLP-1-based therapeutic material, studies have been focused on dual agonists binding to both GLP-1 receptors and glucagon receptors. These were shown to be more effective in body weight reduction due to the activation of glucagon receptors, compared to when GLP-1 was used alone (Jonathan W et al., Nat Chem Bio., 2009 October (5); 749-757).

In a study related to triple agonists, which bind to GLP-1, GIP, and glucagon receptors simultaneously, efforts have been made to increase the half-life of the triple agonists by substituting an amino acid sequence to increase the resistance to dipeptidyl peptidase-IV (DPP-IV), which decomposes gastrointestinal hormones to get rid of their activities, followed by adding an acyl group to a particular region thereof (Finan B et al., Nat Med., 2015 21 (1): 27-36). However, their effects of activating three different kinds of receptors were not significant and no triple agonist showed various active ratios thereto.

Therefore, it is an object of the invention to provide compositions and methods for treating or preventing one or more symptoms of metabolic diseases including type 2 diabetes mellitus, dyslipidemia, metabolic syndrome, non-alcoholic fatty liver disease, non-alcoholic steatohepatitis, and/or obesity.

It is also an object of the invention to provide compositions and methods suitable for activating GLP-1, GIP, and glucagon receptors to control blood glucose levels and reduce body weight without causing side effects.

It is a further object of the invention to provide compositions and methods that provide effective glucose control, with weight loss benefits and a favorable side effect profile.

It is a further object of the present invention to provide therapeutic agents with extended duration of action.

### SUMMARY OF THE INVENTION

Triple agonist peptides having activities at each of GLP-1, glucagon and GIP receptors are provided. Generally, the triple agonist peptides have the following Polypeptide Formula I

(SEQ ID NO: 131)
X₁X₂X₃GTFTSDX₁₀SX₁₂X₁₃LDX₁₆X₁₇X₁₈X₁₉X₂₀X₂₁X₂₂X₂₃X₂₄X₂₅X₂₆X₂₇
X₂₈G X₃₀X₃₁SX₃₃X₃₄X₃₅PP X₃₈ X₃₉X₄₀,

wherein X₁ is H or Y;
X₂ is A or 2-aminoisobutyric acid (Aib);
X₃ is Q or E;
X₁₂ is R, W or K;
X₁₃ is L or Y;
X₁₉ is Q, A or T;
X₂₁ is D or L;
X₂₂ is F or R;
X₂₃ is V, G or D;
X₂₅ is W, Y or A;
X₂₆ is L or D;
X₂₇ is I, L, M, G or P;
X₃₀ is G or P;
X₃₁ is P or S;
X₃₃ is S or G;
X₃₄ is G or A;
X₃₅ is A or P;
X₃₈ is P or S;
X₃₉ is absent, S, or C;
X₄₀ is absent, C, or K;
with an optional amide modification of the C-terminus and
X₁₀, X₁₆, X₁₇, X₁₈, X₂₀, X₂₄, and X₂₈, is independently any one of the 20 amino acids except cysteine. In some embodiments, X₁₇, X₁₈, X₂₀ are non-natural amino acids. In some embodiments, where X₁₇, X₁₈, X₂₀ are non-natural amino acids, X₁₇, X₁₈, X₂₀ is independently one of the following: methoxinine, 2-aminoisobutyric acid, and alpha-methyl-arginine.

In other embodiments:
X₁₀ is Y, W, K, F, H, S, L, A, E, M, Q or D;
X₁₆ is Y, Q, G, K, S, R, F, P or A;
X₁₇ is M, Y, Q, K, S, W, P, D, A, F or methoxinine;
X₁₈ is A, I, M, W, T, D, Y or methoxinine;
X₂₀ is R, Q, H, G, A, P, N, K, Aib or alpha-methyl-arginine;
X₂₄ is Q, D, K, L, N, W or M; and
X₂₈ is N, E, G, D, H or Q.

In preferred embodiments, the triple agonist peptides have amino acid sequence of any one of SEQ ID NOs. 1-130.

In one embodiment, the triple agonist peptide does not have the amino acid sequence of SEQ ID NO: 134: YXQGTFTSDYSKLLDYMMQRDFVQWLLEGGPSSGAPPPSK (SEQ ID NO: 134), where X is any one of the 20 amino acids.

In one embodiment, the triple agonist peptide does not have the amino acid sequence of SEQ ID NO: 134:

(SEQ ID NO: 135)
YAibQGTFTSDYSKLLDYMMQRDFVQWLLEGGPSSGAPPPSK.

Triple agonist analogs having one or more biotin moieties and/or fatty acid moieties conjugated thereto for improved bioavailability and pharmacokinetics are also described. In some embodiments, the one or more biotin moieties and/or one or more fatty acids, or derivatives thereof, are conjugated to the amino acid sequence of any one of SEQ ID NOs. 1-130 via one or more amino acid residues selected from the group consisting of cysteine and lysine. In other embodiments, one or more amino acid residues of cysteine and lysine are introduced to the amino acid sequence of any one of SEQ ID NOs. 1-130 by substitution or insertion to allow conjugation to the one or more biotin moieties and/or one or more fatty acids, or derivatives thereof. In preferred embodiments, one or more amino acid residues of lysine at position 10, lysine at position 12, lysine at position 17, one or more C-terminal cysteine residues are introduced to the amino acid sequence of any one of SEQ ID NOs. 1-130 by substitution or insertion to allow conjugation to the one or more biotin moieties and/or one or more fatty acids, or derivatives thereof. Exemplary biotin moieties suitable for conjugation are N-Biotinoyl-N′-(6-maleimidohexanoyl) hydrazide, 3-Maleimidopropionate-Lys(Biotin)-Lys(Biotin)-CONH₂, 3-Maleimidopropionate-Lys(Biotin)-Lys(Biotin)-Lys(Biotin)-CONH₂, propionate-N-hydroxysuccinimide ester-PEG-Lys(Biotin)-Lys(Biotin)-Lys(Biotin)-CONH₂ and 3-Maleimidopropionate-PEG-Lys(Biotin)-Lys(Biotin)-Lys(Biotin)-CONH₂.

Exemplary fatty acids suitable for conjugation are C16-C22 fatty acids, optionally via one or more hydrophilic spacers such as γGlu or 8-amino-3,6-dioxaoctanoic acid. In some embodiments, the fatty acids or derivatives thereof suitable for conjugation are C16-NHS, C16-MAL, C18-NHS, C18-MAL, C16-γGlu-NHS, C16-γGlu-MAL, C18-γGlu-NHS, C18-γGlu-MAL, C18-γGlu-OEG-NHS, C18-γGlu-OEG-MAL, C18-γGlu-2OEG-NHS, C18-γGlu-2OEG-MAL, C20-γGlu-2OEG-NHS, C20-γGlu-2OEG-MAL, C18-γGlu-2OEG-TFP, C18-γGlu-2OEG-NPC, and C20-γGlu-2OEG-NPC. Pharmaceutical formulations the triple agonist peptide or analog thereof, and methods of use thereof are also described.

Methods of treating one or more diseases selected from the group consisting of obesity, diabetes, and non-alcoholic fatty liver disease in a subject in need thereof are provided. The methods include administering an effective amount of the pharmaceutical formulation of the triple agonist peptide or analog thereof to treat or alleviate one or more symptom of the one or more diseases. In preferred embodiments, the pharmaceutical formulation is administered in an amount effective to induce weight loss, reduce body fat, reduce food intake, improve glucose homeostasis, or combinations thereof, in a normal or obese patient. In some embodiments, the subject is suffering from non-alcoholic fatty liver disease (NAFLD), for example, non-alcoholic fatty liver, non-alcoholic steatohepatitis, liver cirrhosis, and liver cancer. In the case of NAFLD, the pharmaceutical formulation is administered in an amount effective to inhibit or reduce serum levels of one or more of alanine aminotransferase, aspartate aminotransferase, triglyceride, gamma-glutamyl transferase, total cholesterol, low density lipoprotein, fasting blood sugar or combinations thereof. In preferred embodiments, the pharmaceutical formulation is administered in an amount effective to reduce one or more of steatosis, inflammation, ballooning, fibrosis, cirrhosis, or combinations thereof, in a subject with NAFLD.

Typically, the pharmaceutical formulation is administered via enteral administration and parenteral administration, for example, oral administration or subcutaneous administration. In some embodiments, the pharmaceutical formulation is administered in a form of pills, capsules, tablets, liquids, and suspensions. In some embodiments, the pharmaceutical formulation is administered at an interval of once a month, once every two weeks, once a week, once every three days, once every two days, once daily, or twice daily. In other embodiments, the pharmaceutical formulation is administered the subject once a week for up to 6 months, or for a duration of between one and 10 days, weeks, months, or years, inclusive. In some embodiments, the pharmaceutical formulation is administered to a human subject at a dose of between 0.001 mg/kg body weight of the subject and 10 mg/kg body weight of the subject, inclusive. In preferred embodiments, the pharmaceutical formulation is administered to a human subject at a dose of between 0.01 mg/kg body weight of the subject and 1 mg/kg body weight of the subject, inclusive. In further embodiments, the pharmaceutical formulation is administered to a human subject at a dose of between 1.0 mg and 100 mg, inclusive.

### BRIEF DESCRIPTION OF THE DRAWINGS

FIG. 1 is amino acid sequence used for peptide design of phage display library.

FIG. 2 is a bar graph showing luminescence in three target cell lines GLP-1R+ cells, GCGR+ cells, GIPR+ cells and two control cell lines (cells HEK293 and CHO cells) in the presence of 1st and 2nd rounds of enriched phage pools (1st-1-P enriched pool, 1st-2-P enriched pool, 1st-3-P enriched pool, 2nd-1-P enriched pool, 2nd-2-P enriched pool, and 2nd-3-P enriched pool) using cAMP activity assay. The unscreened library phage and helper phage M13KO7 were also used as control phages. A PBS control was also included.

FIGS. 3A-3C are bar graph showing luminescence in three target cell lines GLP-1R+ cells (FIG. 3A), GCGR+ cells (FIG. 3B), and GIPR+ cells (FIG. 3C) using 18 unique peptides identified from phage display, with C-terminal cysteine.

FIGS. 4A-4C are schematic illustration of conjugations of a biotin moiety, a fatty acid moiety, or a biotin and fatty acid moiety to triple agonist peptides. These modifications are termed A type (lipid at Cys40, FIG. 4A), B type (lipid at Cys40 and biotin at Lys12, FIG. 4B) and C type (lipid at Lys12 and biotin at Cys40, FIG. 4C).

FIGS. 5A-5C are bar graphs showing luminescence in three target cell lines GLP-1R+ cells (FIG. 5A), GCGR+ cells (FIG. 5B), and GIPR+ cells (FIG. 5C) using 18 unique peptides, identified from phage display, with lipidated at C-terminal cysteine at four different concentrations of 1 nM, 10 nM, 100 nM, and 300 nM.

FIGS. 6A-6B are line graphs showing body weight change over a period of 4 days in grams (FIG. 6A) or as a percentage (FIG. 6B) of starting body weight in mice treated with vehicle, peptide 5A, 12A administered at 20 nmol/kg, s.c. QD or 30 nmol/kg, s.c. Q2D, peptide DD01 (administered at 40 nmol/kg, s.c. Q2D) and Semaglutide (30 nmol/kg, s.c. Q2D) as positive controls.

FIGS. 7A-7C are bar graphs showing percentage (%) cAMP release after incubation with peptide Nos. 5-1, 5-2, 5-3, 5-4, 5-5, 5-6, 12-1, 12-2, 12-3, 12-4, 12-5, 12-6, and 12-7 as well as the respective native ligands (GLP-1, glucagon, and GIP) as a positive control at five different concentrations of 0.3 nM, 1 nM, 10 nM, 100 nM, and 1000 nM, in GLP-1R+ cells (FIG. 7A), GCGR+ cells (FIG. 7B), and GIPR+ cells (FIG. 7C). FIG. 7A shows percentage (%) cAMP release relative cAMP release of GLP-1 at 10 nM and EC₅₀ values for GLP-1 is 0.227 nM. FIG. 7B shows percentage (%) cAMP release relative cAMP release of glucagon at 100 nM and EC₅₀ values for glucagon is 1.271 nM. FIG. 7C shows percentage (%) cAMP release relative cAMP release of GIP at 33 nM and EC₅₀ values for GIP is 1.784 nM.

FIGS. 8A-8C are line graphs showing c-AMP release at a concentration range between 0.1 nM and 1000 nM of peptide Nos. 5-1A, 5-2A, 5-3A, 5-4A, 5-5A, and 5-6A as well as the respective native ligands (GLP-1, glucagon, and GIP) as a positive control in three target cell lines GLP-1R+ cells (FIG. 8A), GCGR+ cells (FIG. 8B), and GIPR+ cells (FIG. 8C). EC₅₀ values for GLP-1, glucagon, and GIP in GLP-1R+ cells, GCGR+ cells, and GIPR+ cells, are 0.09 nM, 0.99 nM, and 0.24 nM, respectively.

FIGS. 9A-9C are line graphs showing c-AMP release at a concentration range between 0.1 nM and 1000 nM of peptide Nos. 12-1A, 12-2A, 12-3A, 12-4A, 12-5A, 12-6A, and 12-7A as well as with their respective native ligands (GLP-1, glucagon, and GIP) as a positive control in three target cell lines GLP-1R+ cells (FIG. 9A), GCGR+ cells (FIG. 9B), and GIPR+ cells (FIG. 9C). EC₅₀ values for GLP-1, glucagon, and GIP in GLP-1R+ cells, GCGR+ cells, and GIPR+ cells, are 0.09 nM, 0.99 nM, and 0.24 nM, respectively.

FIGS. 10A-10B are line graphs showing body weight change over a period of 4 days in grams (FIG. 10A) or as a percentage of starting body weight (FIG. 10B) in mice treated with peptides 5A, 5-1A, 5-2A, 5-3A, 5-4A, and 5-5A administered at 20 nmol/kg, s.c. QD. Peptide DD01 (administered at 40 nmol/kg, s.c. Q2D) as positive controls.

FIGS. 11A-11D are line graphs showing changes in body weight over a period of 3 days in grams (FIG. 11A) or as a percentage of starting body weight (FIG. 11B), changes in blood glucose % (FIG. 11C), and changes in food intake (FIG. 11D), in mice treated with peptides 5A, 12-1A, 12-2A, 12-3A, 12-4A, 12-5A, and 12-7A administered at 20 nmol/kg, s.c. QD.

FIGS. 12A-12C are bar graphs showing cAMP release after incubation with peptide Nos. 5A, 5-1A, 5D, 5-1D, 5-2D, 5-3D, 5-4D, 5-5D, 5-1E, and 5-1F, as well as the respective native ligands (GLP-1, glucagon, and GIP) as a positive control at five different concentrations of 0.3 nM, 1 nM, 10 nM, 100 nM, and 300 nM or 1000 nM, in GLP-1R+ cells (FIG. 12A), GCGR+ cells (FIG. 12B), and GIPR+ cells (FIG. 12C).

FIGS. 13A-13D are line graphs showing changes in body weight over a period of 4 days in grams (FIG. 13A) or as a percentage of starting body weight (FIG. 13B), changes in blood glucose % (FIG. 13C), and changes in food intake (FIG. 13D), in mice treated with peptides 5D, 5-1D, 5-2D, 5-3D, 5-4D, 5-5D, 5-1E, and 5-1F administered at 20 nmol/kg, s.c. QD.

FIGS. 14A-14G are line graphs showing changes in body weight over a period of 2 weeks in grams (FIG. 14A) or as a percentage of starting body weight (FIG. 14B), changes in blood glucose % (FIG. 14C), liver weight (FIG. 14D), triglyceride content (FIG. 14E), as well as mRNA levels of TGF-β1 (FIG. 14F) and ACTA2 (FIG. 14G) in mAMLN mice treated with peptides 5-1A, 5D, 5-2D, 5-1D, and 5-3D as well as positive controls DD01 and EL.EX.14. CHOW and vehicle controls are also included. Mean SEM, *p vs. G1, ^{#}p vs. G2. One-way ANOVA, Turkey's multiple comparison test.

FIG. 15A-15C are bar graphs showing cAMP release after incubation with SEQ ID NOs. 77, 84, 85, 86, 87, 88, 89, 90, 91, 92, 93, 94, 95, and 96, as well as the respective native ligands (GLP-1, glucagon, and GIP) as a positive control at five different concentrations between 0.01 nM and 100 nM in GLP-1R+ cells (FIG. 15A), GCGR+ cells (FIG. 15B), and GIPR+ cells (FIG. 15C).

FIG. 16A-16C are bar graphs showing cAMP release after incubation with SEQ ID NOs. 66, 97, and 98, as well as the respective native ligands (GLP-1, glucagon, and GIP) as a positive control at five different concentrations between 0.01 nM and 100 nM in GLP-1R+ cells (FIG. 16A), GCGR+ cells (FIG. 16B), and GIPR+ cells (FIG. 16C).

FIG. 17A-17C are graphs showing changes in body weight over a period of 2 weeks in grams (FIG. 17A), changes in blood glucose % (FIG. 17B), and cumulative food intake from baseline following 2 weeks (FIG. 17C) in HFD mice treated with SEQ ID NOs. 77, 86, 87, 89, and 90. Vehicle control is also included. Mean SEM, ^{#}p vs. G1. One-way ANOVA, Dunnett's multiple comparisons test.

FIG. 18 is graph showing half-lives determined by remaining amount of SEQ ID NOs. 77, 86, 87, 89, 90, and 91 against FaSSIF/P.

FIG. 19A-19D are graphs showing changes in body weight over a period of 4 weeks in grams (FIG. 19A), and levels of ALT (FIG. 19B), AST (FIG. 19C), and LDL (FIG. 19D) in CDA-HFD mice treated with SEQ ID NOs. 77, 87, 89, 90, 95, and 96. CHOW and vehicle controls are also included. Mean SEM, *p vs. G2. One-way ANOVA, Dunnett's multiple comparisons test.

FIG. 20A-20C are bar graphs showing cAMP release after incubation with SEQ ID NOs. 77, 89, 90, 99, 100, 101, 102, 103, 104, and 105 as well as with the respective native ligands (GLP-1, glucagon, and GIP) as a positive control at five different concentrations between 0.01 nM and 100 nM in GLP-1R+ cells (FIG. 20A), GCGR+ cells (FIG. 20B), and GIPR+ cells (FIG. 20C).

FIG. 21A-21B are graphs showing half-lives determined by remaining amount of SEQ ID NOs. 77, 99, 100, and 101 against FaSSIF/P (FIG. 21A) and trypsin (FIG. 21B).

FIG. 22A-22F are graphs showing changes in body weight over a period of 4 weeks in grams (FIG. 22A), levels of serological parameters including ALT (FIG. 22B), AST (FIG. 22C), and LDL (FIG. 22D), triglyceride content (FIG. 22E), as well as NAFLD activity score (NAS) (FIG. 22F) in CDA-HFD mice treated with SEQ ID NOs. 77, 99, 100, 101, 102, and 103. CHOW and vehicle controls are also included. Mean SEM, *p vs. G2. One-way ANOVA, Dunnett's multiple comparisons test; ^{%}p, G7 vs G8, ^{$}p, G3 vs G7, ^{&}p, G3 vs G8, Unpaired two-tailed Student's test.

FIG. 23A-23H are graphs showing changes in body weight over a period of 4 weeks in grams (FIG. 23A), liver weight and liver to body weight ratio (FIG. 23B), visceral fat weight and visceral fat to body weight ratio (FIG. 23C), levels of serological parameters including ALT (FIG. 23D), AST (FIG. 23E), and LDL (FIG. 23F), triglyceride content (FIG. 23G), as well as histology score of steatosis, lobular inflammation, and ballooning (FIG. 23H) in mAMLN mice treated with SEQ ID NOs. 77, 89, 97, 104, and 101 as well as tirzepatide as a positive control. CHOW and vehicle controls are also included. Mean SEM, *p vs. G2, #p vs. G8, One-way ANOVA, Dunnett's multiple comparisons test.

FIG. 24A-24C are graphs showing cAMP release after incubation with SEQ ID NOs. 66, 97, 101, 106, 107, 108, 109, 110, 111, and 112 as well as with the respective native ligands (GLP-1, glucagon, and GIP) and tirzepatide as a positive control at six different concentrations between 0.001 nM and 100 nM in GLP-1R+ cells (FIG. 24A), GCGR+ cells (FIG. 24B), and GIPR+ cells (FIG. 24C).

FIG. 25A-25G are graphs showing changes in body weight over a period of 4 weeks in grams (FIG. 25A), liver weight and liver to body weight ratio (FIG. 25B), levels of serological parameters including ALT (FIG. 25C), AST (FIG. 25D), and LDL) (FIG. 25E), triglyceride content (FIG. 25F) as well as NAFLD activity score (FIG. 25G) in CDA-HFD mice treated with SEQ ID NOs. 97, 110, 108, 111 and 112 as well as tirzepatide as a positive control. CHOW and vehicle controls are also included. Mean SEM, *p vs. G2, ^{$}p vs. G8, One-way ANOVA, Dunnett's multiple comparisons test.

### DETAILED DESCRIPTION OF THE INVENTION

### I. Definitions

The term “therapeutic agent” refers to an agent that can be administered to treat one or more symptoms of a disease or disorder. The term “prophylactic agent” generally refers to an agent that can be administered to prevent disease or to prevent certain conditions.

The term “pharmaceutically acceptable salt”, as used herein, refers to derivatives of the compounds defined herein, wherein the parent compound is modified by making acid or base salts thereof. Example of pharmaceutically acceptable salts include, but are not limited to, mineral or organic acid salts of basic residues such as amines; and alkali or organic salts of acidic residues such as carboxylic acids. Pharmaceutically acceptable salts include the conventional non-toxic salts or the quaternary ammonium salts of the parent compound formed, for example, from non-toxic inorganic or organic acids. Such conventional non-toxic salts include those derived from inorganic acids such as hydrochloric, hydrobromic, sulfuric, sulfamic, phosphoric, and nitric acids; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, tolunesulfonic, naphthalenesulfonic, methanesulfonic, ethane disulfonic, oxalic, and isethionic salts.

The phrase “pharmaceutically acceptable” or “biocompatible” refers to compositions, polymers, and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The phrase “pharmaceutically acceptable carrier” refers to pharmaceutically acceptable materials, compositions, or vehicles, such as a liquid or solid filler, diluent, solvent, or encapsulating material involved in carrying or transporting any subject composition, from one organ, or portion of the body, to another organ, or portion of the body. Each carrier must be “acceptable” in the sense of being compatible with the other ingredients of a subject composition and not injurious to the patient.

The term “therapeutically effective amount” refers to an amount of the therapeutic agent that produces some desired effect at a reasonable benefit/risk ratio applicable to any medical treatment. The effective amount may vary depending on such factors as the disease or condition being treated, the size of the subject, or the severity of the disease or condition. One of ordinary skill in the art may empirically determine the effective amount of a particular compound without necessitating undue experimentation. In some embodiments, the term “effective amount” refers to an amount of a prophylactic agent or therapeutic agent to reduce or diminish the risk of developing a liver disease/disorder or to reduce or diminish one or more symptoms of a liver disease/disorder, such as reducing inflammation in the liver. Additional desired results also include reducing and/or inhibiting serum levels of alanine aminotransferase (ALT), aspartate aminotransferase (AST), triglyceride (TG) and total cholesterol (TC), fat accumulation or steatosis, inflammation, ballooning, fibrosis, long-term morbidity and mortality. An effective amount can be administered in one or more administrations.

The terms “inhibit” or “reduce” in the context of inhibition, mean to reduce or decrease in activity and quantity. This can be a complete inhibition or reduction in activity or quantity, or a partial inhibition or reduction. Inhibition or reduction can be compared to a control or to a standard level. Inhibition can be 5, 10, 25, 50, 75, 80, 85, 90, 95, 99, or 100%. For example, long-lasting GLP-1r agonists may inhibit or reduce the activity and/or quantity of activated microglia by about 10%, 20%, 30%, 40%, 50%, 75%, 85%, 90%, 95%, or 99% from the activity and/or quantity of the same cells in equivalent tissues of subjects that did not receive or were not treated with long-lasting GLP-1r agonists. In some embodiments, the inhibition and reduction are compared at mRNAs, proteins, cells, tissues, and organs levels.

The term “treating” or “treatment” refers to amelioration, alleviation or reduction of one or more symptoms of a disease, disorder, or condition in a person who may be predisposed to the disease, disorder and/or condition but has not yet been diagnosed as having it; reducing disease symptoms, inhibiting the disease, disorder or condition, e.g., impeding its progress; and relieving the disease, disorder, or condition, e.g., causing regression of the disease, disorder and/or condition. Treating the disease or condition includes ameliorating at least one symptom of the particular disease or condition, even if the underlying pathophysiology is not affected, such as treating the pain of a subject by administration of an analgesic agent even though such agent does not treat the cause of the pain. Desirable effects of treatment include decreasing the rate of disease progression, ameliorating, or palliating the disease state, and remission or improved prognosis. For example, an individual is successfully “treated” if one or more symptoms associated with liver diseases/disorders are mitigated or eliminated, including, but not limited to, reducing and/or inhibiting elevations of the transaminases including alanine transaminase (ALT) and aspartate transaminase (AST), reducing the proliferation of cancerous cells in the case of liver cancer, increasing the quality of life of those suffering from the disease, decreasing the dose of other medications required to treat the disease, delaying the progression of the disease, and/or prolonging survival of individuals. The term “ameliorate” refers to a decrease, suppression, attenuation, diminish, arrest, or stabilization of the development or progression of a disease.

The terms “prevent”, “prevention” or “preventing” mean to administer a composition or method to a subject or a system at risk for or having a predisposition for one or more symptom caused by a disease or disorder, to decrease the likelihood the subject will develop one or more symptoms of the disease or disorder, or to reduce the severity, duration, or time of onset of one or more symptoms of the disease or disorder.

The term “biodegradable” generally refers to a material that will degrade or erode under physiologic conditions to smaller units or chemical species that are capable of being metabolized, eliminated, or excreted by the subject. The degradation time is a function of composition and morphology.

The terms “protein” or “polypeptide” or “peptide” refer to any chain of more than two natural or unnatural amino acids, regardless of post-translational modification (e.g., glycosylation or phosphorylation), constituting all or part of a naturally occurring or non-naturally occurring polypeptide or peptide.

The terms “biotinylation” and “biotinylated” refer to the process and product of both covalent attachment of one or more biotin moieties or derivatives thereof to molecules and macrostructures, such as a therapeutic protein.

The terms “lipidation” and “lipidated” refer to the process and product of both covalent attachment of one or more fatty acid moieties or derivatives thereof to molecules and macrostructures, such as a therapeutic protein.

The term “PEGylation” refers to a process of both covalent and non-covalent attachment or amalgamation of polyethylene glycol (PEG) polymer chains to molecules and macrostructures, such as a drug, therapeutic protein or vesicle.

Use of the term “about” is intended to describe values either above or below the stated value in a range of approx. +/−10%; in other embodiments the values may range in value either above or below the stated value in a range of approx. +/−5%.

### II. Compositions

Compositions including isolated peptides having activities to a glucagon receptor, a glucagon-like peptide-1 (GLP-1) receptor, and a glucose-dependent insulinotropic polypeptide (GIP) receptor, are provided.

### A. Triple Agonist Peptides

In some embodiments, the triple agonist peptides have the amino acid sequence of the following Polypeptide Formula (I):

(SEQ ID NO: 131)
X₁X₂X₃GTFTSDX₁₀SX₁₂X₁₃LDX₁₆X₁₇X₁₈X₁₉X₂₀X₂₁X₂₂X₂₃X₂₄X₂₅X₂₆X₂₇
X₂₈G X₃₀X₃₁SX₃₃X₃₄X₃₅PP X₃₈ X₃₉X₄₀,

wherein X₁ is H or Y;
X₂ is A or 2-aminoisobutyric acid (Aib);
X₃ is Q or E;
X₁₂ is R, W or K;
X₁₃ is L or Y;
X₁₉ is Q, A or T;
X₂₁ is D or L;
X₂₂ is F or R;
X₂₃ is V, G or D;
X₂₅ is W, Y or A;
X₂₆ is L or D;
X₂₇ is I, L, M, G or P;
X₃₀ is G or P;
X₃₁ is P or S;
X₃₃ is S or G;
X₃₄ is G or A;
X₃₅ is A or P;
X₃₈ is P or S;
X₃₉ is absent, S, or C;
X₄₀ is absent, C, or K;
with an optional amide modification of the C-terminus and
X₁₀, X₁₆, X₁₇, X₁₈, X₂₀, X₂₄, and X₂₈, any one of the 20 amino acids except cysteine. In some embodiments, X₁₇, X₁₈, X₂₀ are non-natural amino acids. In some embodiments, where X₁₇, X₁₈, X₂₀ are non-natural amino acids, X₁₇, X₁₈, X₂₀ is independently one of the following: methoxinine, 2-aminoisobutyric acid, and alpha-methyl-arginine.

In other embodiments,
X₁₀ is Y, W, K, F, H, S, L, A, E, M, Q or D;
X₁₆ is Y, Q, G, K, S, R, F, P or A;
X₁₇ is M, Y, Q, K, S, W, P, D, A, F or methoxinine;
X₁₈ is A, I, M, W, T, D, Y or methoxinine;
X₂₀ is R, Q, H, G, A, P, N, K, Aib or alpha-methyl-arginine;
X₂₄ is Q, D, K, L, N, W or M; and
X₂₈ is N, E, G, D, H or Q.

In one embodiment, the triple agonist peptide does not have the amino acid sequence of SEQ ID NO: 134: YXQGTFTSDYSKLLDYMMQRDFVQWLLEGGPSSGAPPPSK (SEQ ID NO: 134), where X is any one of the 20 amino acids.

In one embodiment, the triple agonist peptide does not have the amino acid sequence of SEQ ID NO: 135:

(SEQ ID NO: 135)
YAibQGTFTSDYSKLLDYMMQRDFVQWLLEGGPSSGAPPPSK.

In some embodiments, Polypeptide Formula (I) has the amino acid sequence of any one of SEQ ID NOs: 1-130.

### 1. Modifications to Triple Agonist Peptides

The direct use of native polypeptides as biopharmaceuticals is often limited by their very short systemic half-lives resulting from a rapid metabolism, enzymatic degradation, and, for smaller proteins and peptides, effective renal clearance. Modifications to exenatide such as semaglutide, liraglutide, and NLY01, have extended the half-life and pharmacokinetics of the active agent. Thus, further modifications are made to further improve the oral bioavailability, stability, and/or pharmacokinetics.

In some embodiments, the triple agonist peptides have the amino acid sequence of any one of SEQ ID NOs: 1-130. In preferred embodiments, the triple agonist peptides are triple agonist analogs modified with one or more biotin moieties and/or one or more fatty acids, optionally with one or more spacers, to achieve desired pharmacokinetics, stability, and bioavailability. In other embodiments, the triple agonist peptides are triple agonist analogs modified with one or more biotin moieties, one or more fatty acids, and/or one or more PEG moieties, optionally with one or more spacers, to achieve desired pharmacokinetics, stability, and bioavailability. In further embodiments, the triple agonist peptides disclosed herein are modified with C-terminal amidation.

The choice of the suitable functional group for modifications is based on the type of available reactive group on the molecule that will be coupled to the biotin moieties and/or fatty acids. Typical reactive amino acids include lysine, cysteine, histidine, arginine, aspartic acid, glutamic acid, serine, threonine, tyrosine. The N-terminal amino group and the C-terminal carboxylic acid can also be used as a site-specific conjugation. In preferred embodiments, the reactive amino acids are lysine and cysteine.

In preferred embodiments, one or more biotin moieties and/or one or more fatty acids, or derivative thereof, are conjugated to the amino acid sequence of any one of SEQ ID NOs: 1-130 via one or more of amino acid residues of lysine at position 10, lysine at position 12, lysine at position 17, lysine at position 20, lysine at position 24, and one or more C-terminal inserted cysteine or lysine residues. In other embodiments, one or more cysteine and lysine residues are introduced via substitution or insertion into the amino acid sequence of any one of SEQ ID NOs: 1-130 to facilitate conjugation to biotin moieties and/or fatty acids, or derivatives thereof. In particular embodiments, the triple agonist peptides have the amino acid sequence of any one of SEQ ID NOs: 1-130.

a. Biotinylation

Biotin modifications to exendin derivatives have been previously described, for example, in International Publication Nos. WO2009107900A1, WO2020242268A1, and WO2021107519A1.

Korean Patent Registration No. 10-0864584 describes an exendin-4 derivative in which biotin is modified in a lysine residue of exendin-4 may be administered orally and has improved bioavailability in the intestine. However, in this case, there is a problem in that biotin is conjugated to various lysine positions of exendin-4 to form various isomers, thereby lowering the reaction rate and yield, and biotin is conjugated to a lysine position of an N-terminal which is an active site of exendin-4 to inhibit the activity of exendin-4.

Accordingly, in preferred embodiments, one or more biotin moieties are conjugated to amino acids (e.g., cysteine or lysine) at suitable positions to provide an excellent oral bioavailability without inhibiting the activity of triple agonist peptides.

In some embodiments, the triple agonist peptides have an improved in vivo oral bioavailability compared to the same triple agonist peptides without the one or more biotin moieties conjugated thereto. In preferred embodiments, the biotin-conjugated triple agonist peptides retain most of the activity of the same triple agonist peptides without the one or more biotin moieties conjugated thereto.

In some embodiments, the biotin moiety conjugated to one or more amino acid residues (e.g., cysteine or lysine) of a triple agonist peptide is represented by the following General Formula A.

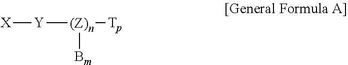

wherein,
X is a functional group capable of being conjugated to the polypeptide,
Y is a spacer,
Z is a binding unit,
B may be represented by the following Chemical Formula A-1,

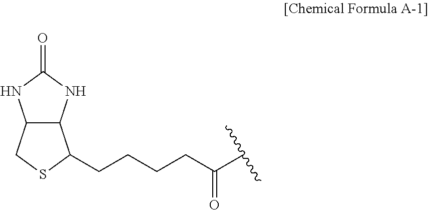 [SMILES: *C(=O)CCCCC1SCC2NC(=O)NC21]

T is a terminal group,
m is an integer of 1 to 10,
n is an integer of 1 to 10, and
p is an integer of 0 or 1.

In some embodiments, when n=0, Y can link directly to B or T.

In some embodiments, the biotin moiety-conjugated polypeptide is a peptide having the amino acid sequence of any one of SEQ ID NOs: 1-130. Alternatively, one or more cysteine or lysine residues are inserted internally at any position within the amino acid sequence of any one of SEQ ID NOs: 1-130 to facilitate the conjugation to biotin.

In some embodiments, the biotin moiety is conjugated to the triple agonist peptides via one or more additional cysteine or lysine residues added to the C-terminus of the polypeptide. In one embodiment, the biotin moiety is conjugated to triple agonist peptides via one additional cysteine residue added to the C-terminus of the amino acid sequence of any one of SEQ ID NOs: 1-130. In another embodiment, the biotin moiety is conjugated to the triple agonist polypeptide via one additional lysine residue added to the C-terminus of the amino acid sequence of any one of SEQ ID NOs: 1-130. In some embodiments, the amino acid of the second position of SEQ ID NOs: 1-18 is substituted with 2-aminoisobutyric acid (Aib). In some embodiments, the biotin moiety is conjugated to the triple agonist polypeptide via one or more internal lysine residues, for example, lysine position 10, lysine at position 12, lysine at position 17, lysine at position 20, and/or lysine at position 24, of any of SEQ ID NOs: 1-130.

In General Formula A representing the biotin moiety, X is a functional group capable of being conjugated with cysteine of the polypeptide. Although not limited thereto, for example, the functional group may be maleimide, amine, succinimide, N-hydroxysuccinimide, aldehyde or carboxyl group, and more specifically maleimide.

In one embodiment, when the functional group X in General Formula A is conjugated with cysteine or lysine of the polypeptide, the structure may be maintained, or removed or modified.

In General Formula A, the Y may be a spacer and may have a structure having cleavability in the body. Although not limited thereto, for example, the Y is a direct-bonded, or substituted or unsubstituted alkylene, wherein the alkylene may include at least one of —O—, —C(═O)NR—, —C(═O)O— or —C(═O)—, —NR—, and —NOR—, and the R may be hydrogen, and substituted or unsubstituted alkyl or aryl.

In one embodiment, the spacer may include a structure represented by the following Formula.

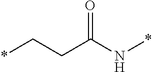 [SMILES: *CCC(=O)N*]

In some embodiments, in General Formula A, the Z is a binding unit capable of binding to B, and may include, for example, an amino acid, a polypeptide, an alkylene amine, or a polyamidoamine structure, but not limited thereto.

Although not limited thereto, for example, the amino acid may be lysine, 5-hydroxylysine, 4-oxallysine, 4-thialysine, 4-selenalysine, 4-thiahomolysine, 5,5-dimethyllysine, 5,5-difluorolysine, trans-4-dehydrolysine, 2,6-diamino-4-hexynoic acid, cis-4-dehydrolysine, 6-N-methyllysine, diminopimelic acid, ornithine, 3-methylornithine, α-methylornithine, citrulline or homocitrulline, arginine, aspartate, asparagine, glutamate, glutamine, histidine, ornithine, proline, serine, or threonine.

When the n is 0, B may directly bind to Y (spacer).

In some embodiments, in General Formula A, the T is a terminal group, and although not limited thereto, may be, for example, hydrogen or NH₂.

When the p is 0, the B may be a terminal.

In one embodiment, in General Formula A above, “m” may be an integer of 1 to 10, and specifically, may be an integer of 1 to 8, 1 to 5, and 1 to 4.

In one embodiment, the biotin moiety may be represented by the following General Formula 1A:

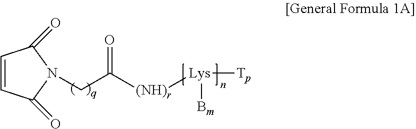

wherein,
Lys is lysine,
T is hydrogen or NH₂,
q is an integer of 1 to 5,
r is an integer of 0, 1 to 3, and
B, n, m, and p are as defined in General Formula A above.

In one embodiment, the biotin moiety may be represented by the following General Formula 2A or 3A:

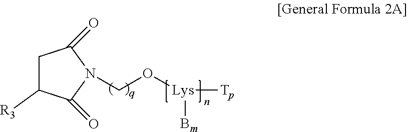

wherein,
Lys is lysine,
T is hydrogen or NH₂,
R₃ is hydrogen or —SO₃—,
q is an integer of 0, or 1 to 4, and
B, n, m, and p are as defined in General Formula A above.

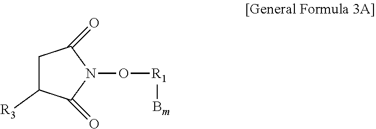

wherein,
R₁ is a direct bond or NH,
R₃ is hydrogen or —SO₃—, and
B and m are as defined in General Formula A above.

In one embodiment, the biotin moiety may be represented by the following structures I-III.

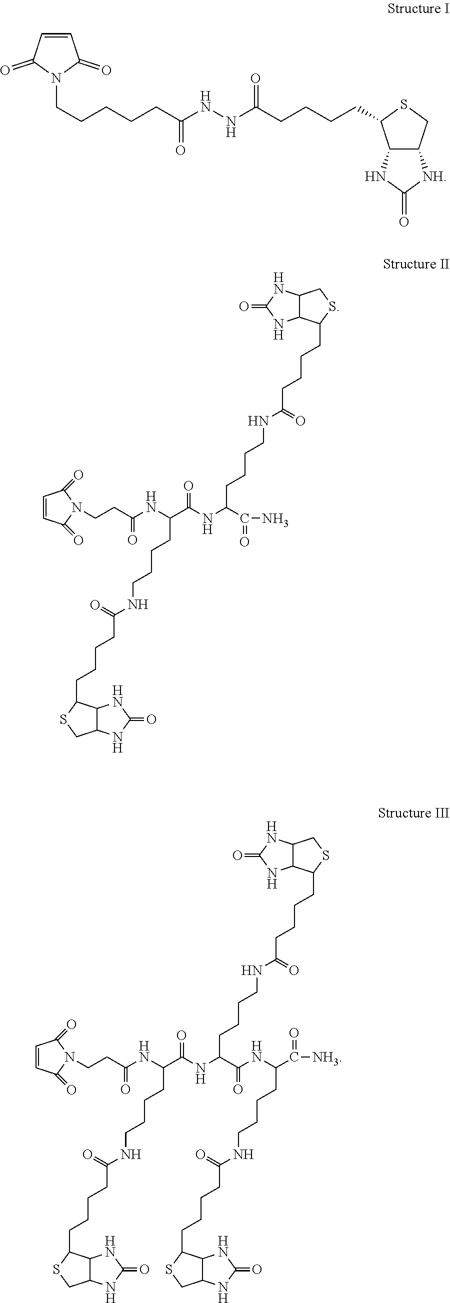

Exemplary biotin derivatives are shown in Tables 1 and 2, below.

TABLE 1
Examples of biotin derivatives.

| Biotin | | |
|---|---|---|
| No. | Structure | Abbreviation |
| B1 | N-Biotinoyl-N′-(6-maleimidohexanoyl)hydrazide | B1-MAL |
| B2 | 3-Maleimidopropionate-Lys(Biotin)-Lys(Biotin)- | B2-MAL |
| | CONH₂ | |
| B3 | 3-Maleimidopropionate-Lys(Biotin)-Lys(Biotin)- | B3-MAL |
| | Lys(Biotin)-CONH₂ | |
| B4 | propionate-N-hydroxysuccinimide ester- PEG- | NHS-PEG- |
| | Lys(Biotin)-Lys(Biotin)-Lys(Biotin)-CONH₂ | B3 |
| B5 | 3-Maleimidopropionate-PEG-Lys(Biotin)- | MAL-PEG- |
| | Lys(Biotin)-Lys(Biotin)-CONH₂ | B3 |

TABLE 2
Examples of biotin derivatives.

| | | | | Number |
|---|---|---|---|---|
| Biotin | | | | of |
| No. | X | Y | Z | Biotin |
| B6 | Aldehyde | propane | Lysine | 2 |
| B7 | Maleimide | butyrate | Glycerol and PEG | 2 |
| B8 | Maleimide | butyrate | Glycerol and PEG | 2 |
| B9 | N-hydroxysuccinimide | butyrate | Lysine | 2 |
| B10 | N-hydroxysuccinimide | glutarate | Glycerol and PEG | 2 |
| B11 | Maleimide | PEG12 | Lysine | 3 |
| B12 | N-hydroxysuccinimide | PEG12 | Lysine | 3 |
| B13 | amine | — | Lysine | 3 |
| B14 | Aldehyde | pentane | Lysine | 2 |
| B15 | Maleimide | adipate | Glycerol and PEG | 2 |
| B16 | Maleimide | suberate | Glycerol and PEG | 2 |
| B17 | Maleimide | sebacate | Glycerol and PEG | 2 |
| B18 | N-hydroxysuccinimide | adipate | Glycerol and PEG | 2 |
| B19 | N-hydroxysuccinimide | suberate | Lysine | 4 |
| B20 | N-hydroxysuccinimide | sebacate | Lysine | 4 |
| B21 | N-hydroxysuccinimide | PEG6 | Glycerol and PEG | 2 |
| B22 | Succinimidyl carbonate | PEG6 | Lysine | 2 |
| B23 | Succinimidyl carbonate | PEG12 | Lysine | 3 |
| B24 | Succinimidyl carbonate | pentane | Lysine | 3 |
| B25 | Succinimidyl carbonate | hexane | Lysine | 3 |
| B26 | p-nitrophenyl carbonate | PEG6 | Lysine | 3 |
| B27 | p-nitrophenyl carbonate | PEG12 | Lysine | 4 |
| B28 | p-nitrophenyl carbonate | propane | Glycerol and PEG | 2 |
| B29 | p-nitrophenyl carbonate | pentane | Glycerol and PEG | 2 |
| B30 | amine | — | Glycerol and PEG | 2 |
| B31 | thiol | butyrate | Lysine | 2 |
| B32 | thiol | glutarate | Lysine | 3 |
| B33 | aminoxy | PEG6 | Lysine | 3 |
| B34 | iodoacetamide | PEG6 | Lysine | 3 |
| B35 | Maleimide | C18-EG2-Glu | Lysine | 3 |
| B36 | Maleimide | C18-EG2-Glu | Lysine | 3 |
| B37 | Amine | EG2-NH—(CH₂)₅—COOH | Lysine | 3 |
| B38 | N-hydroxysuccinimide | — | Lysine | 1 |
| B39 | N-hydroxysuccinimide | — | Lysine | 1 |
| B40 | Maleimide | — | | 1 |

B39 and B40 use desthiobiotin, an analog of biotin, as shown in Structure IV.

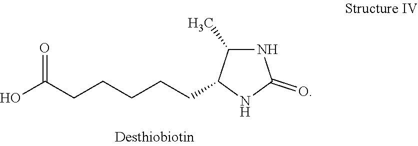

In some embodiments, biotin analog NHS-desthiobiotin as shown in Structure V, is used for conjugation.

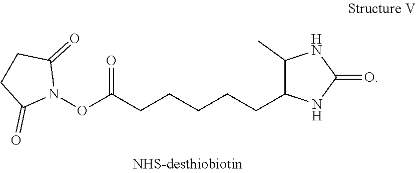 [SMILES: CC1NC(=O)NC1CCCCCC(=O)ON1C(=O)CCC1=O]

b. Lipidation

Lipidated peptides have an increased lipophilicity, an increased in vivo half-life (enabling once daily oral administration), and a reduced variability in pharmacokinetics at steady state. In some embodiments, an additional amino acid is added to the C-terminus of the triple agonist peptide to enable the conjugation of one or more fatty acid molecules with increased linker stability. In preferred embodiments, the amino acid is either cysteine or lysine.

In some embodiments, the fatty acid moiety is conjugated to the triple agonist peptides via one or more additional cysteine or lysine residues added to the C-terminus of the polypeptide. In one embodiment, the fatty acid moiety is conjugated to the triple agonist peptides via one additional cysteine residue added to the C-terminus of the amino acid sequence of SEQ ID NOs: 1-130. In some embodiments, the amino acid of the second position of SEQ ID NOs: 1-18 is substituted with 2-aminoisobutyric acid (Aib). In some embodiments, the fatty acid moiety is conjugated to the triple agonist polypeptide via one or more internal lysine residues, for example lysine at position 10, lysine at position 12, lysine at position 17, and/or lysine at position 20, lysine at position 24 of any of SEQ ID NOs: 1-130.

The first lipidated biopharmaceutical to obtain regulatory approval was insulin detemir in 2004. Insulin detemir, a basal insulin for the treatment of diabetes, includes desB30 human insulin conjugated to myristic acid (C14) through the Ne-amine of LysB29.

Current lipidated biopharmaceuticals have hydrophilic spacers, typically γGlu and/or OEG (8-amino-3,6-dioxaoctanoic acid), in between the lipid and peptide moieties to increase parameters such as albumin affinity, potency, water-solubility, and oligomerization. One example is liraglutide, a once-daily glucagon-like peptide 1 (GLP-1) analog marketed for treatment of diabetes and obesity. The liraglutide sequence is identical to that of native GLP-1 except for a Lys34Arg substitution, which enables selective palmitoylation through the Na of Lys26 via a γGlu spacer (Lau J.; et al., J. Med. Chem. 2015, 58 (18), 7370-7380). Because of albumin binding and slow absorption, liraglutide has a significantly extended half-life (11-15 h, s.c.) compared to native GLP-1 (1-1.5 h, s.c.). From the dietary fatty acids used in insulin detemir and liraglutide, the preferred fatty acid for lipidation has advanced to the nondietary dicarboxylic fatty acids used in insulin degludec, a once-daily basal insulin, and semaglutide, a once-weekly GLP-1 analog. Insulin degludec is lipidated at LysB29 with a γGlu-spaced palmitic diacid. The peptide backbone of Semaglutide is similar to liraglutide's, except for a substitution of Alanine8 to 2-aminoisobutyric acid (Aib), which reduces degradation by dipeptidyl peptidase IV (DPP-4) (Lau, J. et al., Journal of Medicinal Chemistry (2015), 58 (18), 7370-7380). Semaglutide is lipidated at Lys26 with an octadecanoic diacid through a spacer including γGlu and two OEG units, which elicits an albumin affinity 5.6-fold larger than liraglutide's. The high albumin affinity as well as the DPP-4 resistance gives Semaglutide a half-life of approximately 1 week in humans (s.c.) (van Witteloostuijn, S. B.; Pedersen, S. L.; Jensen, K. J. Chem Med Chem 2016, 11, 1-23). Impressively, this prolonged half-life is obtained without decreasing the GLP-1 receptor potency compared to the native ligand. Recently, lipidation has also been shown as a viable strategy for half-life extension of larger proteins as demonstrated by somapacitan, a once-weekly human growth hormone. The lipidation of somapacitan includes a significantly longer spacer region and a noncarboxylic fatty acid with a tetrazole headgroup.

Thus, in some embodiments, the triple agonist peptide is conjugated to one or more of fatty acid chains, preferably with one or more hydrophilic spacers, such as γGlu or OEG (8-amino-3,6-dioxaoctanoic acid), in between the lipid and peptide. Exemplary fatty acids can include dietary fatty acids such as those used in insulin detemir and liraglutide, and the preferred fatty acids for lipidation are non-dietary dicarboxylic fatty acids such as those used in insulin degludec and semaglutide.

Exemplary fatty acid derivatives are shown in Table 3, below.

TABLE 3
Examples of fatty acid derivatives

| Fatty acid No. | Structure |
|---|---|
| F1 | C16-NHS |
| F2 | C16-MAL |
| F3 | C18-NHS |
| F4 | C18-MAL |
| F5 | C16-γGlu-NHS |
| F6 | C16-γGlu-MAL |
| F7 | C18-γGlu-NHS |
| F8 | C18-γGlu-MAL |
| F9 | C18-γGlu-OEG-NHS |
| F10 | C18- γGlu-OEG-MAL |
| F11 | C18-γGlu-2OEG-NHS |
| F12 | C18-γGlu-2OEG-MAL |
| F13 | C20-γGlu-2OEG-NHS |
| F14 | C20-γGlu-2OEG-MAL |
| F15 | C18-γGlu-2OEG-TFP |
| F16 | C18-γGlu-2OEG-NPC |
| F17 | C20-γGlu-2OEG-NPC |

In preferred embodiments, the triple agonist peptides are lipidated and/or biotinylated. In particular embodiments, the triple agonist peptides have the amino acid sequence of any one of SEQ ID NOs: 1-130 and modified with at one or more sites as described listed in Table 23 (A, B, C, D, E, and F) using one or more biotin derivatives listed in Tables 1 and 2, and/or one or more fatty acid derivatives listed in Table 3.

c. Pegylation

In some embodiments, triple agonist peptides are modified with polyethylene glycol, polyethyl imine, or derivatives thereof. In preferred embodiments, triple agonist peptides are modified by PEGylation at similar sites to biotinylation and lipidation sites. Modifications can alter pharmacokinetics, pharmacodynamics, stability, and bioavailability.

Polyethylene glycol (PEG) is a polyether compound with many applications from industrial manufacturing to medicine. The structure of PEG is (note the repeated element in parentheses): H—(O—CH₂—CH₂)ₙ—OH. PEG is also known as polyethylene oxide (PEO) or polyoxyethylene (POE), depending on its molecular weight. PEG, PEO, or POE refers to an oligomer or polymer of ethylene oxide. The three names are chemically synonymous, but historically PEG is preferred in the biomedical field, whereas PEO is more prevalent in the field of polymer chemistry. Because different applications require different polymer chain lengths, PEG has tended to refer to oligomers and polymers with a molecular mass below 20,000 g/mol, PEO to polymers with a molecular mass above 20,000 g/mol, and POE to a polymer of any molecular mass. PEG and PEO are liquids or low-melting solids, depending on their molecular weights. PEGs are prepared by polymerization of ethylene oxide and are commercially available over a wide range of molecular weights from 300 g/mol to 10,000,000 g/mol. While PEG and PEO with different molecular weights find use in different applications, and have different physical properties (e.g., viscosity) due to chain length effects, their chemical properties are nearly identical. Different forms of PEG are also available, depending on the initiator used for the polymerization process 2—the most common initiator is a monofunctional methyl ether PEG, or methoxypoly(ethylene glycol), abbreviated mPEG. Lower-molecular-weight PEGs are also available as purer oligomers, referred to as monodisperse, uniform, or discrete. Very high purity PEG has recently been shown to be crystalline, allowing determination of a crystal structure by x-ray diffraction. Since purification and separation of pure oligomers is difficult, the price for this type of quality is often 10-10,00 fold that of polydisperse PEG.

PEGs are also available with different geometries. Branched PEGs have three to ten PEG chains emanating from a central core group. Star PEGs have 10 to 100 PEG chains emanating from a central core group. Comb PEGs have multiple PEG chains normally grafted onto a polymer backbone. The numbers that are often included in the names of PEGs indicate their average molecular weights (e.g., a PEG with n=9 would have an average molecular weight of approximately 400 Daltons and would be labeled PEG 400. Most PEGs include molecules with a distribution of molecular weights (i.e., they are polydisperse). The size distribution can be characterized statistically by its weight average molecular weight (Mw) and its number average molecular weight (Mn), the ratio of which is called the polydispersity index (Mw/Mn). MW and Mn can be measured by mass spectrometry.

In some embodiments, the polyethylene glycol or a derivative thereof is a linear type or a branched type, and for the branched type, preferably a dimeric type or a trimeric type may be used, and more preferably a trimeric type may be used. Specifically, the polyethylene glycol derivative is, for example, methoxypolyethylene glycol succinimidylpropionate, methoxypolyethylene glycol N-hydroxysuccinimide, methoxypolyethylene glycol propionaldehyde, methoxypolyethylene glycol maleimide, or multiple branched types of these derivatives. Preferably, the polyethylene glycol derivative is linear methoxypolyethylene glycol maleimide, branch type methoxypolyethylene glycol maleimide or trimeric methoxypolyethylene glycol maleimide, and more preferably is trimeric methoxypolyethylene glycol maleimide.

PEG is a particularly attractive polymer for conjugation. The specific characteristics of PEG moieties relevant to pharmaceutical applications are: water solubility, high mobility in solution, lack of toxicity and low immunogenicity, ready clearance from the body, and altered distribution in the body.

PEGylation (also often styled pegylation) is the process of both covalent and non-covalent attachment or amalgamation of polyethylene glycol (PEG) polymer chains to molecules and macrostructures, such as a drug, therapeutic protein or vesicle, which is then described as PEGylated (pegylated). PEGylation is routinely achieved by incubation of a reactive derivative of PEG with the target molecule. The covalent attachment of PEG to a drug or therapeutic protein can “mask” the agent from the host's immune system (reduced immunogenicity and antigenicity), and increase the hydrodynamic size (size in solution) of the agent which prolongs its circulatory time by reducing renal clearance. PEGylation can also provide water solubility to hydrophobic drugs and proteins.

PEGylation can improve the safety and efficiency of many therapeutics such as peptides, proteins, and antibody fragments. It produces alterations in the physiochemical properties including changes in conformation, electrostatic binding, hydrophobicity etc. These physical and chemical changes increase systemic retention of the therapeutic agent. Also, it can influence the binding affinity of the therapeutic moiety to the cell receptors and can alter the absorption and distribution patterns.

PEGylation increases molecular weight, defense of a metabolism site and inhibition of an immunogenicity site, increasing in vivo half-life and stability and reducing immunogenicity. Furthermore, kidney excretion of peptides and proteins bound with PEG is reduced due to the increase of molecular weights of peptides and proteins by PEG, so that PEGylation has advantages of increasing effects in both pharmacokinetically and pharmacodynamically.

In some embodiments, the triple agonist peptides are modified with polyethylene glycol, polyethyl imine, or derivatives thereof. In preferred embodiments, the triple agonist peptides are modified by PEGylation at similar sites to biotinylation and lipidation sites. After the triple agonist peptide is PEGylated with polyethylene glycol or the derivative thereof is prepared, the molecular structure of the analogue may be confirmed by a mass spectroscope, a liquid chromatography, an X-ray diffraction analysis, a polarimetry, and comparison between calculated values and measured values of representative elements constituting the PEGylated triple agonist peptide.

In some embodiments, a PEG or a derivative thereof is conjugated to the triple agonist peptides via one or more additional cysteine or lysine residues added to the C-terminus of the polypeptide. In one embodiment, a PEG or a derivative thereof is conjugated to the triple agonist peptides via one additional cysteine residue added to the C-terminus of the amino acid sequence of SEQ ID NOs: 1-53. In some embodiments, the amino acid of the second position of SEQ ID NOs: 1-53 is substituted with 2-aminoisobutyric acid (Aib). In some embodiments, a PEG or a derivative thereof is conjugated to the triple agonist polypeptide via one or more internal lysine residues, for example, lysine at position 10, lysine at position 12, lysine at position 17, lysine at position 20, and/or lysine at position 24 of any of SEQ ID NOs: 1-64.

### B. Pharmaceutical Formulations

In some embodiments, the triple agonist peptides or analogs thereof are formulated with one or more pharmaceutical excipients, additives, or fillers. For example, in some embodiments, the triple agonist peptides or analogs thereof are formulated into pharmaceutical formulations for administration to a subject. Compositions including triple agonist peptides or analogs thereof may be formulated in a conventional manner using one or more physiologically acceptable carriers including excipients and auxiliaries which facilitate processing of the active compounds into preparations which can be used pharmaceutically.

Proper formulation is dependent upon the route of administration chosen. In preferred embodiments, the compositions are formulated for parenteral delivery. In preferred embodiments, the compositions are formulated for subcutaneous delivery. In some embodiments, the compositions are formulated for intravenous injection. Typically, the compositions will be formulated in sterile saline or buffered solution for injection into the tissues or cells to be treated. The compositions can be stored lyophilized in single use vials for rehydration immediately before use. Other means for rehydration and administration are known to those skilled in the art.

Pharmaceutical formulations contain the triple agonist peptides or analogs thereof in combination with one or more pharmaceutically acceptable excipients. Representative excipients include solvents, diluents, pH modifying agents, preservatives, antioxidants, suspending agents, wetting agents, viscosity modifiers, tonicity agents, stabilizing agents, and combinations thereof. Suitable pharmaceutically acceptable excipients are preferably selected from materials which are generally recognized as safe (GRAS), and may be administered to an individual without causing undesirable biological side effects or unwanted interactions.

Generally, pharmaceutically acceptable salts can be prepared by reaction of the free acid or base forms of an agent with a stoichiometric amount of the appropriate base or acid in water or in an organic solvent, or in a mixture of the two; generally, non-aqueous media like ether, ethyl acetate, ethanol, isopropanol, or acetonitrile are preferred. Pharmaceutically acceptable salts include salts of an agent derived from inorganic acids, organic acids, alkali metal salts, and alkaline earth metal salts as well as salts formed by reaction of the drug with a suitable organic ligand (e.g., quaternary ammonium salts). Lists of suitable salts are found, for example, in Remington's Pharmaceutical Sciences, 20th ed., Lippincott Williams & Wilkins, Baltimore, MD, 2000, p. 704. Examples of ophthalmic drugs sometimes administered in the form of a pharmaceutically acceptable salt include timolol maleate, brimonidine tartrate, and sodium diclofenac.

### 1. Dosage Units

The compositions are preferably formulated in dosage unit form for ease of administration and uniformity of dosage. The phrase “dosage unit form” refers to a physically discrete unit of conjugate appropriate for the patient to be treated. It will be understood, however, that the total single administration of the compositions will be decided by the attending physician within the scope of sound medical judgment. The therapeutically effective dose can be estimated initially either in cell culture assays or in animal models, usually mice, rats, rabbits, dogs, or pigs. The animal model is also used to achieve a desirable concentration range and route of administration. Such information should then be useful to determine useful doses and routes for administration in humans.

### 2. Formulations for Administration

In some embodiments, the compositions of triple agonist peptides or analogs thereof are formulated into a pharmaceutically acceptable formulation for administration via a specific route. In some embodiments, the compositions are administered locally, for example, by injection directly into a site to be treated. In some embodiments, the compositions are injected, topically applied, or otherwise administered directly into the vasculature onto vascular tissue at or adjacent to a site of injury, surgery, or implantation. For example, in some embodiments, the compositions are topically applied to vascular tissue that is exposed, during a surgical procedure. Typically, local administration causes an increased localized concentration of the compositions, which is greater than that which can be achieved by systemic administration.

Pharmaceutical compositions formulated for administration by parenteral (intramuscular, intraperitoneal, intravenous (IV) or subcutaneous injection) and enteral routes of administration are described.

a. Enteral Administration

In some embodiments, the triple agonist peptides or analogs thereof are administered orally. For oral administration, suitable formulations include tablets, pellets, hard/soft capsules, liquids, suspensions, emulsifiers, syrups, granules, elixirs, troches, etc., and these formulations can include diluents (for example, lactose, dextrose, sucrose, mannitol, sorbitol, cellulose and/or glycine), slip modifiers (for example, silica, talc, stearate and its magnesium or calcium salt and/or polyethylene glycol) in addition to the active ingredient. Tablets may also include binders such as magnesium aluminum silicate, starch paste, gelatin, methyl cellulose, sodium carboxymethyl cellulose and/or polyvinyl pyrrolidine, and may include disintegrating agents such as starch, agar, alginic acid or sodium salt thereof or boiling mixture and/or absorbents, coloring agents, flavoring agents and sweetening agents if needed.

In preferred embodiments, one or more absorption or permeation enhancers are used for oral formulation. Exemplary permeation enhancers include bile acid, cholic acid, deoxycholic acid, glycocholic acid, glycochonodeoxycholic acid, taurochenodeoxycholic acid, taurocholic acid, chenodeoxycholic acid, ursodeoxycholic acid, lithocholic acid, Labrasol (Caprylocaproyl Polyoxyl-8 glycerides), SNAC (sodium N-(8-[2-hydroxybenzoyl]amino) caprylate, propyl gallate and their salt forms.

b. Parenteral Administration

In some embodiments, the triple agonist peptides or analogs thereof are formulated into a pharmaceutically acceptable formulation for parenteral administration. The phrases “parenteral administration” and “administered parenterally” are art-recognized terms, and include modes of administration other than enteral and topical administration, such as injections, and include without limitation intravenous (i.v.), intramuscular (i.m.), intraperitoneal (i.p.), subcutaneous (s.c.) injection and infusion. The long-acting GLP-1r agonists can be administered parenterally, for example, by intravenous, intraperitoneal, or subcutaneous routes.

For liquid formulations, pharmaceutically acceptable carriers may be, for example, aqueous or non-aqueous solutions, suspensions, emulsions, or oils. Parenteral vehicles (for subcutaneous, intravenous, intraarterial, or intramuscular injection) include, for example, sodium chloride solution, Ringer's dextrose, dextrose and sodium chloride, lactated Ringer's and fixed oils. Examples of non-aqueous solvents are propylene glycol, polyethylene glycol, and injectable organic esters such as ethyl oleate. Aqueous carriers include, for example, water, alcoholic/aqueous solutions, cyclodextrins, emulsions or suspensions, including saline and buffered media. The long-acting triple agonists can also be administered in an emulsion, for example, water in oil. Examples of oils are those of petroleum, animal, vegetable, or synthetic origin, for example, peanut oil, soybean oil, mineral oil, olive oil, sunflower oil, fish-liver oil, sesame oil, cottonseed oil, corn oil, olive, petrolatum, and mineral. Suitable fatty acids for use in parenteral formulations include, for example, oleic acid, stearic acid, and isostearic acid. Ethyl oleate and isopropyl myristate are examples of suitable fatty acid esters.

Formulations suitable for parenteral administration can include antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Intravenous vehicles can include fluid and nutrient replenishers, electrolyte replenishers such as those based on Ringer's dextrose. In general, water, saline, aqueous dextrose and related sugar solutions, and glycols such as propylene glycols or polyethylene glycol are preferred liquid carriers, particularly for injectable solutions.

Injectable pharmaceutical carriers for injectable compositions are well-known to those of ordinary skill in the art (see, e.g., Pharmaceutics and Pharmacy Practice, J.B. Lippincott Company, Philadelphia, PA, Banker and Chalmers, eds., pages 238-250 (1982), and ASHP Handbook on Injectable Drugs, Trissel, 15th ed., pages 622-630 (2009)).

### III. Methods of Making

### A. Methods of Making Triple Agonist Peptide Analogs

Triple agonists can be prepared and modified via a variety of chemical reaction steps. Typically, methods for modifying the triple agonists include biotinylation and/or lipidation. In some embodiments, biotinylation and lipidation can be applied during peptide synthesis (assembly of amino acid by solid-phase-peptide synthesis) using biotin conjugated amino acids or lipid conjugated amino acids.

### 1. Biotinylation and Lipidation

Biotin modifications to exendin derivatives have been previously described, for example, in International Publication Nos. WO2009107900A1, WO2020242268A1, and WO2021107519A1.

In some embodiments, biotinylation and lipidation are carried out simultaneously. In one embodiment, the methods include dissolving the peptide, C18-γGlu-2OEG-MAL (F12), and biotin-N-hydroxysuccinimide ester (B1-NHS, B38) in dimethyl sulfoxide (DMSO) containing 0.3% triethylamine (TEA) (v/v) solution. In one embodiment, the peptide and F12 are mixed at a volume ratio of 1:1. An exemplary concentration of peptide is 5 mg/mL, and the molar ratio is 1:2 (peptide:lipid). In one embodiment, the mixture is reacted at 25° C. for 10 min with gently shaking. In another embodiment, B38 is added at a volume ratio of 1:0.2 and the molar ratio is 1:3 (peptide: biotin). In one embodiment, the mixture is reacted at 25° C. for 60 min with gently shaking.

In another embodiment, the methods include dissolving the peptide, biotin-maleimide (B1-MAL, B1), and C18-γGlu-2OEG-NPC (F16) in DMSO containing 0.3% TEA (v/v) solution. In one embodiment, the peptide and B1 is mixed at a volume ratio of 1:1. An exemplary concentration of peptide is 5 mg/mL, and the molar ratio is 1:2 (peptide: biotin). In one embodiment, the mixture is reacted at 25° C. for 10 min with gently shaking, and then, F16 is added at a volume ratio of 1:0.2 and the molar ratio is 1:2 (peptide:lipid). In one embodiment, the mixture is reacted at 25° C. for 90 min with gently shaking. Lipidated and biotinylated peptides can be purified by Prep-LC and the eluate can be collected in individual fractions. In one embodiment, the ACN contained in the fractionated solution is evaporated using the centrifugal evaporator at 45° C. for 40 min. The solvent can be changed to water by ultrafiltration. The purified samples can be analyzed by reversed phase-HPLC for purity check. In one embodiment, the samples are lyophilized at −88° C. for 18 hr and then stored at −20° C.

### 2. Lipidation

In one embodiment, the methods include dissolving the peptide and C18-γGlu-2OEG-MAL (F12) in DMSO containing 0.3% TEA (v/v) solution; and mixing each solution at a volume ratio of 1:1. In one embodiment, the concentration of peptide is 5 mg/mL, and the molar ratio is 1:2 (peptide:lipid). The mixture is then reacted at 25° C. for 30 min with gently shaking. In one embodiment, lipidated peptides is purified by Prep-LC and the eluate is collected in individual fractions. In one embodiment, the ACN contained in the fractionated solution is evaporated using the centrifugal evaporator at 45° C. for 40 min. The solvent can be changed to water by ultrafiltration. The purified samples can then be analyzed by reversed phase-HPLC for purity check. In one embodiment, the samples are lyophilized at −88° C. for 18 hr and then stored at −20° C.

### IV. Methods of Use

Pharmaceutical formulations containing one or more of the triple agonist peptides as described herein or analogs thereof can be administered to a subject in need thereof to treat or prevent one or more diseases.

### A. Methods of Treatment

Methods of using triple agonist peptides for treating or preventing one or more metabolic diseases are described. Methods are effective in treating or preventing one or more metabolic diseases such as dyslipidemia, fatty liver disease, metabolic syndrome, nonalcoholic fatty liver disease (NAFLD), obesity and type 2 diabetes mellitus (T2DM), preferably with minimal side effects.

In preferred embodiments, the triple agonist peptides or analogs thereof are administered in an amount and with a dosing regimen effective to prevent, inhibit, or reduce one or more symptoms associated with one or more metabolic diseases such as dyslipidemia, fatty liver disease, metabolic syndrome, NAFLD, obesity and T2DM in the subject. In preferred embodiments, the disease is fatty liver disease, obesity, NAFLD or T2DM.

The triple agonist peptides or analogs thereof are administered to a subject in one or multiple doses, at one or multiple time points following an initial dose. The amount of compositions administered to the subject is selected to deliver an effective amount to reduce, prevent, or otherwise alleviate one or more clinical or molecular symptoms of the disease or disorder to be treated compared to a control, for example, a subject treated with an agonist primarily targeting a single receptor of GLP-1 glucagon, or GIP receptors. The compositions and methods are also suitable for prophylactic use.

Methods are also suitable for treating or preventing one or more neurodegenerative diseases such as Alzheimer's Disease (AD) or Parkinson's disease. In some embodiments, methods include a step of administering to an individual in need thereof an effective amount of a triple agonist peptide or analog thereof.

### B. Conditions to be Treated

The compositions and formulations of these triple agonist peptides are effective to alleviate or prevent one or more symptoms of metabolic diseases or neurological diseases with minimal off-target toxicity or side effects.

In some embodiments, the subject to be treated is a human. In some embodiments, the subject to be treated is a child, or an infant. All the methods can include the step of identifying and selecting a subject in need of treatment, or a subject who would benefit from administration with the described compositions.

### 1. Obesity and Type 2 Diabetes Mellitus

Obesity is a medical condition in which excess body fat has accumulated to the extent that it may have an adverse effect on health, leading to reduced life expectancy and/or increased health problems. Body mass index (BMI), a measurement which compares weight and height, defines people as overweight (pre-obese or overweight) if their BMI is between 25 and 30 kg/m², and obese when it is greater than 30 kg/m².

Obesity increases the risk of many physical and mental disorders. Excessive body weight is associated with various diseases, particularly cardiovascular diseases, diabetes mellitus type 2, obstructive sleep apnea, certain types of cancer, and osteoarthritis. These diseases are either directly caused by obesity or indirectly related through mechanisms sharing a common cause such as a poor diet and/or a sedentary lifestyle. One of the strongest links is with type 2 diabetes. Excess body fat underlies 64% of cases of diabetes in men and 77% of cases in women. Increases in body fat alter the body's response to insulin, potentially leading to insulin resistance.

Methods to treat and/or prevent one or more symptoms of obesity and/or T2DM include administering to a subject in a need thereof an effective amount of a composition to treat and/or alleviate one or more symptoms associated with obesity and/or T2DM. In some embodiments, the compositions or pharmaceutical formulations thereof are administered in an amount effective to induce weight loss, reduce body fat, reduce food intake, improve glucose homeostasis, prevent weight gain, and/or prevent an increase in body mass index in a normal, or obese patient, or combinations thereof.

In some embodiments, the pharmaceutical formulations are administered to a patient suffering from obesity, an obesity-related disease or disorder, diabetes, insulin-resistance syndrome, nonalcoholic steatohepatitis, a cardiovascular disease, or a metabolic syndrome.

In some embodiments, the pharmaceutical formulations are administered to normalize blood sugar; the formulations are preferably administered in an amount effective to lower blood glucose levels to less than about 180 mg/dL. The formulations can be co-administered with other anti-diabetic therapies, if necessary, to improve glucose homeostasis.

Pharmaceutical formulations may also be administered to patients suffering from a disease or disorder that causes obesity or predisposes a patient to become obese.

### 2. Non-Alcoholic Fatty Liver Disease (NAFLD)

In preferred embodiments, the methods and compositions are used to treat or prevent non-alcoholic steatohepatitis, liver fibrosis associated with non-alcoholic steatohepatitis, primary biliary cholangitis.

In some embodiments, the compositions are used to treat nonalcoholic fatty liver disease (NAFLD). NAFLD represents a clinico-pathological spectrum of disease that primarily manifests as excessive accumulation of fat in the hepatocyte (steatosis). NAFLD encompasses the entire spectrum of diseases ranging from simple steatosis to non-alcoholic steatohepatitis (NASH), which can lead to life-threatening hepatic cirrhosis and hepatocellular carcinoma in its most severe form. It is considered to be the hepatic manifestation of the metabolic syndrome, whose other pathologies include obesity, insulin resistance, hypertension and hyperlipidemia. Histologically, NASH is characterized by hepatic steatosis and signs of intralobular inflammation with ballooning degeneration of the hepatocytes. The estimated prevalence of NASH is much lower than NAFLD and ranges from 3 to 5%. Twenty percent of NASH patients are reported to develop cirrhosis, and 30-40% of patients with NASH cirrhosis experience a liver related death.

NAFLD is broadly categorized into 2 phenotypes, namely: non-alcoholic fatty liver (NAFL) which is marked by isolated steatosis, while the more aggressive subtype, non-alcoholic steatohepatitis (NASH), is characterized by cell injury, inflammatory cell infiltration and hepatocyte ballooning that may further progress to fibrosis, cirrhosis, and hepatocellular carcinoma (HCC). In some embodiments, the compositions are used in an amount effective for treating or ameliorating one or more symptoms of non-alcoholic steatohepatitis (NASH).

NAFLD shows a close association with the metabolic syndromes including obesity, type II diabetes, dyslipidemia, and the like based on insulin resistance. In fact, it is known that many pre-diabetic and type II diabetic patients have shown to present with non-alcoholic fatty liver/non-alcoholic steatohepatitis, and the rate of progression to liver cirrhosis and liver cancer (i.e., hepatocellular carcinoma) is high in these patients. Meanwhile, the prevalence of diabetes in non-alcoholic fatty liver disease patients is high, and it is evident in non-alcoholic steatohepatitis patients.

The non-alcoholic fatty liver disease may include one or more diseases selected from the group consisting of non-alcoholic fatty liver, non-alcoholic steatohepatitis, liver cirrhosis, and liver cancer.

In some embodiments, the compositions are administered in an amount effective to prevent the transformation of NAFLD into NASH and to improve the pathophysiology of the disease.

Methods to treat and/or prevent one or more symptoms of NAFLD or NASH typically include administering to a subject in a need thereof an effective amount of a composition to treat and/or alleviate one or more symptoms associated with NAFLD or NASH.

In some embodiments, the compositions are administered in an amount effective to inhibit or reduce serum levels of alanine aminotransferase (ALT), aspartate aminotransferase (AST), triglyceride (TG) and total cholesterol (TC), fat accumulation or steatosis, inflammation, ballooning, fibrosis, long-term morbidity, and mortality.

### 3. Neurological and Neurodegenerative Diseases

The compositions and formulations thereof can be used to treat one or more neurological and neurodegenerative diseases. The compositions and methods are particularly suited for treating one or more neurological, or neurodegenerative diseases associated with activation of microglia and/or astrocytes. In some embodiments, the disease or disorder is selected from, but not limited to, neurological disorders (e.g., Alzheimer's disease (AD), Parkinson's disease (PD)). In one embodiment, the compositions are used to treat Alzheimer's Disease (AD) or Parkinson's disease.

Neurodegenerative diseases are chronic progressive disorders of the nervous system that affect neurological and behavioral function and involve biochemical changes leading to distinct histopathologic and clinical syndromes (Hardy H, et al., Science. 1998; 282:1075-9). Abnormal proteins resistant to cellular degradation mechanisms accumulate within the cells. The pattern of neuronal loss is selective in the sense that one group gets affected, whereas others remain intact. Often, there is no clear inciting event for the disease. The diseases classically described as neurodegenerative are Alzheimer's disease, Huntington's disease, and Parkinson's disease.

Neuroinflammation, mediated by activated microglia and astrocytes, is a major hallmark of various neurological disorders making it a potential therapeutic target. Multiple scientific reports suggest that mitigating neuroinflammation in early phase by targeting these cells can delay the onset of disease and can in turn provide a longer therapeutic window for the treatment (Dommergues, M A et al., Neuroscience 2003, 121, 619; Perry, V H et al., Nat Rev Neurol 2010, 6, 193; Kannan, S et al., Sci. Transl. Med. 2012, 4, 130ra46; and Block, M L et al., Nat Rev Neurosci 2007, 8, 57). The delivery of therapeutics across blood brain barrier is a challenging task. The neuroinflammation causes disruption of blood brain barrier (BBB). The impaired BBB in neuroinflammatory disorders can be utilized to transport drug loaded nanoparticles across the brain (Stolp, H B et al., Cardiovascular Psychiatry and Neurology 2011, 2011, 10; and Ahishali, B et al., International Journal of Neuroscience 2005, 115, 151).

The compositions and methods can also be used to for the treatment of a neurological or neurodegenerative disease or disorder or central nervous system disorder. In preferred embodiments, the compositions and methods are effective in treating, and/or alleviating neuroinflammation associated with a neurological or neurodegenerative disease or disorder or central nervous system disorder. The methods typically include administering to the subject an effective amount of the composition to increase cognition or reduce a decline in cognition, increase a cognitive function or reduce a decline in a cognitive function, increase memory or reduce a decline in memory, increase the ability or capacity to learn or reduce a decline in the ability or capacity to learn, or a combination thereof.

Neurodegeneration refers to the progressive loss of structure or function of neurons, including death of neurons. For example, the compositions and methods can be used to treat subjects with a disease or disorder, such as Parkinson's Disease (PD) and PD-related disorders, Huntington's Disease (HD), Amyotrophic Lateral Sclerosis (ALS), Alzheimer's Disease (AD) and other dementias, Prion Diseases such as Creutzfeldt-Jakob Disease, Corticobasal Degeneration, Frontotemporal Dementia, HIV-Related Cognitive Impairment, Mild Cognitive Impairment, Motor Neuron Diseases (MND), Spinocerebellar Ataxia (SCA), Spinal Muscular Atrophy (SMA), Friedreich's Ataxia, Lewy Body Disease, Alpers' Disease, Batten Disease, Cerebro-Oculo-Facio-Skeletal Syndrome, Corticobasal Degeneration, Gerstmann-Straussler-Scheinker Disease, Kuru, Leigh's Disease, Monomelic Amyotrophy, Multiple System Atrophy, Multiple System Atrophy With Orthostatic Hypotension (Shy-Drager Syndrome), Multiple Sclerosis (MS), Neurodegeneration with Brain Iron Accumulation, Opsoclonus Myoclonus, Posterior Cortical Atrophy, Primary Progressive Aphasia, Progressive Supranuclear Palsy, Vascular Dementia, Progressive Multifocal Leukoencephalopathy, Dementia with Lewy Bodies (DLB), Lacunar syndromes, Hydrocephalus, Wernicke-Korsakoff's syndrome, post-encephalitic dementia, cancer and chemotherapy-associated cognitive impairment and dementia, and depression-induced dementia and pseudodementia. In preferred embodiments, the disease or disorder is Alzheimer's Disease (AD) or Parkinson's disease.

Criteria for assessing improvement in a particular neurological factor include methods of evaluating cognitive skills, motor skills, memory capacity or the like, as well as methods for assessing physical changes in selected areas of the central nervous system, such as magnetic resonance imaging (MRI) and computed tomography scans (CT) or other imaging methods. Such methods of evaluation are well known in the fields of medicine, neurology, psychology and the like, and can be appropriately selected to diagnosis the status of a particular neurological impairment. To assess a change in Alzheimer's disease, or related neurological changes, the selected assessment or evaluation test, or tests, are given prior to the start of administration of the compositions. Following this initial assessment, treatment methods for the administration of the compositions are initiated and continued for various time intervals. At a selected time interval subsequent to the initial assessment of the neurological defect impairment, the same assessment or evaluation test(s) is again used to reassess changes or improvements in selected neurological criteria.

The individual is preferably an adult human, and more preferably, a human is over the age of 30, who has lost some amount of neurological function as a result of Alzheimer's disease or dementia. Generally, neural loss implies any neural loss at the cellular level, including loss in neurites, neural organization, or neural networks.

In other embodiments, the methods including selecting a subject who is likely to benefit from treatment with the compositions.

The compositions and formulations are suitable for reducing or preventing one or more pathological processes associated with the development and progression of PD. Thus, methods for treatment, reduction, and prevention of the pathological processes associated with PD include administering the compositions in an amount and dosing regimen effective to reduce microglial activation, abnormal accumulation of alpha-synuclein protein, neurofibrillary tangles in brains, and/or improved shaking, rigidity, slowness of movement and difficulty with walking, in an individual suffering from PD are provided. Methods for reducing, preventing, or reversing the motor dysfunction in an individual suffering from PD are provided. The methods include administering an effective amount of a composition including one or more long-acting GLP-1r agonists to a subject in need thereof. In preferred embodiments, the methods include administering an effective amount of a composition including one or more triple agonist peptides having amino acid sequence of any one of SEQ ID NOs: 1-64, or pharmaceutically acceptable salt thereof to the subject.

### C. Dosage and Effective Amounts

Dosage and dosing regimens are dependent on the severity of the disorder and/or methods of administration, and can be determined by those skilled in the art. A therapeutically effective amount of triple agonist peptides, or pharmaceutical formulation thereof used in the treatment of fatty liver disease, metabolic syndrome, nonalcoholic fatty liver disease (NAFLD), obesity and/or type 2 diabetes mellitus (T2DM) is typically sufficient to reduce or alleviate one or more symptoms associated with the disease or disorder.

Preferably, the compositions do not target or otherwise modulate the activity or quantity of healthy cells not within or associated with the diseased or target tissues, or do so at a reduced level compared to target cells. In this way, by-products and other side effects associated with the compositions are reduced.

The actual effective amounts can vary according to factors including the specific agent administered, the particular composition formulated, the mode of administration, and the age, weight, condition of the subject being treated, as well as the route of administration and the disease or disorder. In some embodiments, the dose of the triple agonist analog, or pharmaceutical formulation thereof can be from about 0.01 to about 100 mg/kg body weight, from about 0.01 mg/kg to about 10 mg/kg, and from about 0.05 mg to about 5 mg/kg body weight. In other embodiments, the dosage is an absolute amount of a triple agonist analog, or pharmaceutical formulation thereof, for a single administration to a subject, such as from about 0.1 mg up to about 100 mg. For example, in some embodiments, the dosage of a triple agonist analog, or pharmaceutical formulation thereof is 0.1 mg, 0.5 mg, 1 mg, 2 mg, 3 mg, 4 mg, 5 mg, 6 mg, 7 mg, 8 mg, 9 mg or 10 mg, or more than 10 mg, for example 20 mg. 30 mg, 40 mg, 50 mg, or 100 mg. In an exemplary 10 embodiment, the dosage of triple agonist analog is 5 mg, administered once a week. Generally, for intravenous injection or infusion, the dosage may be lower than for oral administration.

In general, the timing and frequency of administration will be adjusted to balance the efficacy of a given treatment schedule with the side-effects of the given delivery system. Exemplary dosing frequencies include continuous infusion, single and multiple administrations such as hourly, daily, weekly, monthly, or yearly dosing.

The triple agonist analog, or pharmaceutical formulation thereof can be administered daily, biweekly, weekly, every two weeks, monthly, or less frequently in an amount to provide a therapeutically effective increase in the blood level of the therapeutic agent. Where the administration is by other than an oral route, the compositions may be delivered over a period of more than one hour, e.g., 3-10 hours, to produce a therapeutically effective dose within a 24-hour period. Alternatively, the compositions can be formulated for controlled release, wherein the composition is administered as a single dose that is repeated on a regimen of once a week, or less frequently.

Dosage can vary, and can be administered in one or more doses daily, once daily, twice weekly, once a week, once every two weeks, once monthly, or less frequently. Guidance can be found in the literature for appropriate dosages for given classes of pharmaceutical products. Optimal dosing schedules can be calculated from measurements of drug accumulation in the body of the subject or patient. Persons of ordinary skill can easily determine optimum dosages, dosing methodologies and repetition rates. Optimum dosages can vary depending on the relative potency of individual pharmaceutical compositions, and can generally be estimated based on EC₅₀s found to be effective in in vitro and in vivo animal models.

In some embodiments, the compositions are administered to a subject for between 1 to 20 years, e.g., 1 year, 2 years, 3 years, 4 years, 5 years, 6 years, 7 years, 8 years, 9 years, 10 years, 11 years, 12 years, 13 years, 14 years, 15 years, 16 years, 17 years, 18 years, 19 years, or 20 years. Optionally, the compositions are administered for 10 years. In one embodiment, the effects of treatment last for at least 1 year.

In preferred embodiments, the triple agonist analog is administered orally in an amount between about 1 mg and about 100 mg, inclusive, preferably between about 5 mg and about 50 mg, inclusive. In some embodiments, the triple agonist peptide or analog thereof is administered orally once a week, once every three days, once every two days, once daily, or twice daily.

In other preferred embodiments, the triple agonist analog is administered parentally such as subcutaneously at a concentration between about 0.1 mg/mL and about 10 mg/mL, inclusive, preferably between about 1 mg/mL and about 5 mg/mL, inclusive. In some embodiments, the triple agonist analog is administered parentally once a week, once every three days, once every two days, or once a day.

In some embodiments, the regimen includes one or more cycles of a round of therapy followed by a drug holiday (e.g., no drug). The drug holiday can be 1, 2, 3, 4, 5, 6, or 7 days, or 1, 2, 3, 4 weeks, or 1, 2, 3, 4, 5, or 6 months.

In some embodiments, the amount of a triple agonist analog administered to a subject changes over time following an initial dose. Therefore, in some embodiments, the amount of a triple agonist analog administered to a subject changes over time following an initial dose.

### D. Combination Therapies and Procedures

The compositions can be administered alone or in combination with one or more conventional therapies. Examples of preferred additional therapeutic agents include other conventional therapies known in the art for treating the desired disease, disorder, or condition. The additional therapeutic, prophylactic or diagnostic agent(s) can have the same or different mechanisms of action. In some embodiments, the combination results in an additive effect on the treatment of the disease or condition. In some embodiments, the combinations result in a more than additive effect on the treatment of the disease or disorder.

### E. Controls

The therapeutic result of the triple agonist analog, or pharmaceutical formulation thereof can be compared to a control or reference. The terms “control” or “reference” refer to a standard of comparison. The term “changed as compared to a control” sample or subject is understood as having a level that is statistically different than a sample from a normal, untreated, or control sample. Control samples include, for example, cells in culture, one or more laboratory test animals, or one or more human subjects. Methods to select and test control samples are within the ability of those in the art. An analyte can be a naturally occurring substance that is characteristically expressed or produced by the cell or organism (e.g., an antibody, a protein) or a substance produced by a reporter construct (e.g., β-galactosidase or luciferase). Depending on the method used for detection, the amount and measurement of the change can vary. Determination of statistical significance is within the ability of those skilled in the art, e.g., the number of standard deviations from the mean that constitute a positive result. Suitable controls are known in the art and include, for example, an untreated subject or untreated cells or the same individual prior to treatment.

### V. Kits

The compositions can be packaged in kit. The kit can include a single dose or a plurality of doses of a composition including one or more of the triple agonist peptides or analogs thereof, or pharmaceutical formulation thereof, and instructions for administering the compositions. In preferred embodiments, the triple agonist peptides or analogs thereof have the amino acid sequence of any one of SEQ ID NOs: 1-130. Specifically, the instructions direct that an effective amount of the composition be administered to an individual with a particular symptoms, disease, defect, or impairment as indicated. The composition can be formulated as described above with reference to a particular treatment method and can be packaged in any convenient manner.

The present invention will be further understood by reference to the following non-limiting examples.

### EXAMPLES

### Example 1: Screening for Triple Agonists Using Phage Display

### Methods

### Screening Using Phage Display

Custom peptide library was constructed using phage display. Phage display technology was used to identify peptides bind to human GLP-1 receptors, human glucagon receptors and human GIP receptors. From phage display, 18 unique peptide sequences were identified. Library screened against target using trimer codon. Trimer codon technology allows making random regions with each amino acid position randomized with a defined amino acid composition as well as region length variation. Also, this method produces less bias. Lead peptide sequences identified after 3-4 rounds of biopanning. Biopanning is an affinity selection technique which selects peptides that binds to a given target.

### Camp Screening for GLP-IR, GCGR, GIPR

To determine the activity of triple agonists in the GLP-1 receptor (GLP-1R)-, Glucagon receptor (GCGR)- and Glucose-dependent insulinotropic polypeptide receptor (GIPR)-expressing CHO-K1 cell line, cell lines are purchased from Eurofins, and HITHUNTER® CAMP Assay for small molecules (DiscoverX) were used.

The cells were seeded in a 96-well plate at 7×10³ cells/well using cell plate reagent and incubated the cells overnight at 37° C. and 5% CO₂. To perform the cAMP detection assay, cell plating reagent were replaced with the cAMP Assay buffer. Each of the wells was then treated with the diluted peptide and incubated for 30 minutes under conditions of a temperature of 37° C. in a CO₂ incubator.

After the incubation, cAMP Antibody reagent and Working cAMP detection solution to all wells. Incubate the plate for 1 hour at Room temperature in the dark. Then, cAMP Solution A was added and incubated for 3 hours at room temperature in the dark. Subsequently, the luminescence was measured using the microplate reader device.

### Results

Phage display technology was used to identify peptides bind to human GLP-1 receptors, human glucagon receptors and human GIP receptors. Phage display technology has been previously described for example in U.S. Pat. No. 10,493,125.

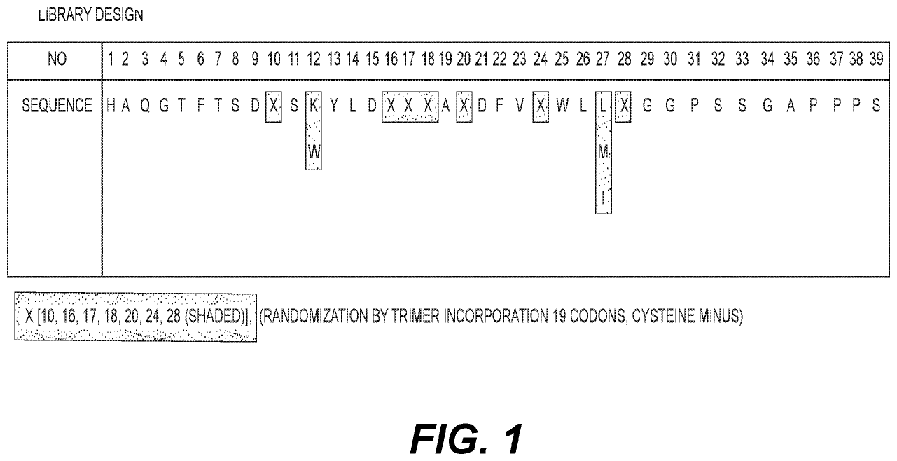

For phage library, the library of peptides was design based on GLP-1, Glucagon and GIP sequence. A custom peptide library was constructed by keeping the hot spot residues the same, while varying other residues as shown in FIG. 1 and Polypeptide Formula II. Amino acid positions at 10, 16, 17, 18, 20, 24, and 28 were randomized by trimer incorporation of 19 codons excluding cysteine. Trimer codon technology allows making random regions with each AA position randomized with a defined AA composition as well as region length variation. Also, this is the best method that produces the least bias. Lead peptide sequences were generated after 3 to 4 rounds of biopanning. Biopanning is an affinity selection technique which selects peptides that are capable of binding to a given target. 1st round cell panning: GLP-1R→GCGR→GIPR; and 2nd round cell panning: GLP-1R→GCGR→GIPR.

The backbone for triple agonists has the amino acid sequence of the following Polypeptide Formula II:

HX₂QGTFTSDX₁₀SX₁₂YLDX₁₆X₁₇X₁₈AX₂₀DFVX₂₄WLX₂₇X₂₈GGPSSGAP
PPSX₄₀,

where X₂ is A or 2-aminoisobutyric acid (Aib);
X₁₂ is K or W;
X₂₇ is L, M, I, G or P;
X₄₀ is absent, C, or K and
X (10, 16, 17, 18, 20, 24, and 28) are randomized by trimer incorporation of 19 codons excluding cysteine.

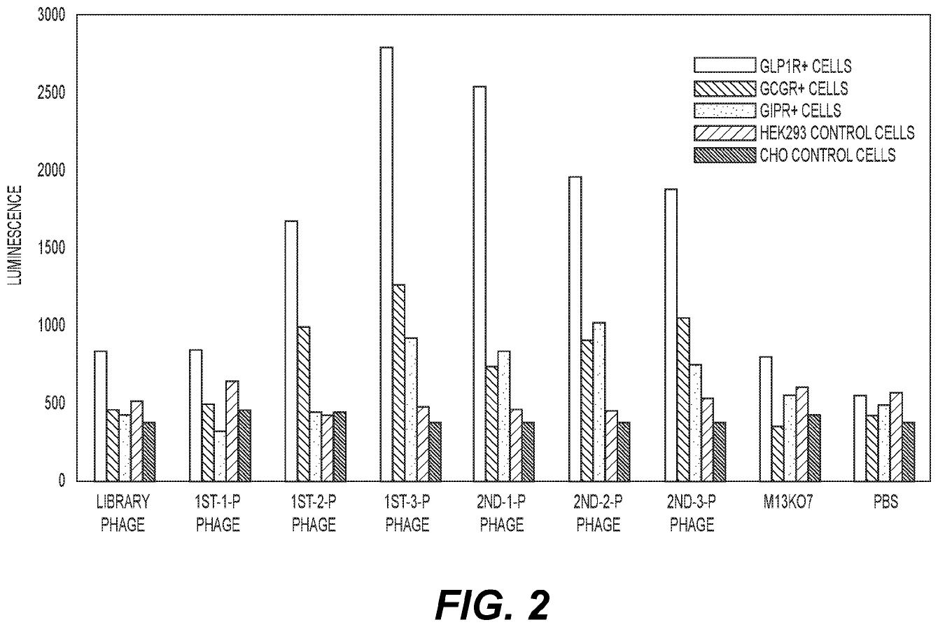

CAMP assay was performed for the 1st and 2nd rounds of enriched phage pools to three target cell lines and two control cell lines. Each enriched pool was tested to its activation to all target cell lines and control cell lines. The unscreened library phage and helper phage M13KO7 were also used as control phages. GLP-1R+ cells were activated from the 1st-2-P enriched pool to 2nd-3-P enriched pool, with the 1st-3-P enriched pool indicated the highest activation effect. GCGR+ cells were also activated from the 1st-2-P enriched pool to 2nd-3-P enriched pool, with the 1st-3-P enriched pool indicated the highest activation effect. GIPR+ cells were activated from the 1st-3-P enriched pool to 2nd-3-P enriched pool, with the 2nd-2-P enriched pool indicated the highest activation effect. There was no apparent activation of six enriched phage pools to the control cells, and no apparent the activation of the control phages to all target cells and control cells (FIG. 2).

Based on the data from cAMP assays, 2nd-2-P enriched pool was selected for carrying out monoclonal phage activation test using cAMP assay kit. From 2nd-2-P phage, monoclonal phage cAMP activity assay validation was conducted. Ninety clones from 2nd-2-P phage were tested, and 29 clones were identified as being able to specifically activate all 3 target cells (GLP-1R+, GCGR+ and GIPR+). After sequencing, 18 unique sequences having activities to all three receptors, GLP-1R, GCGR and GIPR, were identified. Amino acid sequences of the 18 unique peptides are listed in Table 4. A C-terminal cysteine was introduced to each of these 18 peptides to allow incorporation of modification such as lipidation and/or biotinylation and the sequences are listed in Table 5.

TABLE 4
Peptide sequences of 18 candidate peptides

| Peptide | | SEQ |
|---|---|---|
| No. | Amino Acid Sequence | ID NO. |
| 3 | HAQGT FTSDF SKYLD GSWAP DFVLW | 1 |
| | LINGG PSSGA PPPS | |
| 5 | HAQGT FTSDY SKYLD YMMAR DFVQW | 2 |
| | LIEGG PSSGA PPPS | |
| 12 | HAQGT FTSDW SKYLD QQMAQ DFVDW | 3 |
| | LINGG PSSGA PPPS | |
| 18 | HAQGT FTSDH SWYLD KWTAH DFVQW | 4 |
| | LINGG PSSGA PPPS | |
| 19 | HAQGT FTSDW SKYLD KPWAG DFVLW | 5 |
| | LLGGG PSSGA PPPS | |
| 25 | HAQGT FTSDS SWYLD SYWAN DFVNW | 6 |
| | LMGGG PSSGA PPPS | |
| 28 | HAQGT FTSDL SKYLD GPWAG DFVWW | 7 |
| | LIEGG PSSGA PPPS | |
| 32 | HAQGT FTSDA SKYLD RDWTG DFVQW | 8 |
| | LIDGG PSSGA PPPS | |
| 44 | HAQGT FTSDE SWYLD FQDAP DFVQW | 9 |
| | LIHGG PSSGA PPPS | |
| 57 | HAQGT FTSDH SKYLD GAWAA DFVMW | 10 |
| | LLDGG PSSGA PPPS | |
| 58* | HAQGT FTSDF SKYLD PFYAG LRGLA | 11 |
| | DGGGP SSGAP PPS | |
| 60 | HAQGT FTSDE SWYLD PWDAD DFVRW | 12 |
| | LMQGG PSSGA PPPS | |
| 62* | HAQGT FTSDD SWYLD PFYAG LRGLA | 13 |
| | DPGGP SSGAP PPS | |
| 67* | HAQGT FTSDH SKYLD PFYAG LRDLA | 14 |
| | LMGGP SSGAP PPS | |
| 68 | HAQGT FTSDM SKYLD GPWAG DFVQW | 15 |
| | LIDGG PSSGA PPPS | |
| 70 | HAQGT FTSDQ SKYLD APWAG DFVMW | 16 |
| | LIDGG PSSGA PPPS | |
| 76 | HAQGT FTSDD SKYLD KPWAP DFVMW | 17 |
| | LLDGG PSSGA PPPS | |
| 77 | HAQGT FTSDK SWYLD GPWAG DFVLW | 18 |
| | LIEGG PSSGA PPPS | |

*These sequences have an amino acid deletion when aligned
to the sequence of Formula I.

TABLE 5
Peptide sequences of 18 candidate peptides
with C-terminal cysteine

| Peptide | | SEQ |
|---|---|---|
| No. | Amino Acid Sequence | ID NO. |
| 3 | HAibQGT FTSDF SKYLD GSWAP DFVLW | 19 |
| | LINGG PSSGA PPPSC | |
| 5 | HAibQGT FTSDY SKYLD YMMAR DFVQW | 20 |
| | LIEGG PSSGA PPPSC | |
| 12 | HAibQGT FTSDW SKYLD QQMAQ DFVDW | 21 |
| | LINGG PSSGA PPPSC | |
| 18 | HAibQGT FTSDH SWYLD KWTAH DFVQW | 22 |
| | LINGG PSSGA PPPSC | |
| 19 | HAibQGT FTSDW SKYLD KPWAG DFVLW | 23 |
| | LLGGG PSSGA PPPSC | |
| 25 | HAibQGT FTSDS SWYLD SYWAN DFVNW | 24 |
| | LMGGG PSSGA PPPSC | |
| 28 | HAibQGT FTSDL SKYLD GPWAG DFVWW | 25 |
| | LIEGG PSSGA PPPSC | |
| 32 | HAibQGT FTSDA SKYLD RDWTG DFVQW | 26 |
| | LIDGG PSSGA PPPSC | |
| 44 | HAibQGT FTSDE SWYLD FQDAP DFVQW | 27 |
| | LIHGG PSSGA PPPSC | |
| 57 | HAibQGT FTSDH SKYLD GAWAA DFVMW | 28 |
| | LLDGG PSSGA PPPSC | |
| 58* | HAibQGT FTSDF SKYLD PFYAG LRGLA | 29 |
| | DGGGP SSGAP PPSC | |
| 60 | HAibQGT FTSDE SWYLD PWDAD DFVRW | 30 |
| | LMQGG PSSGA PPPSC | |
| 62* | HAibQGT FTSDD SWYLD PFYAG LRGLA | 31 |
| | DPGGP SSGAP PPSC | |
| 67* | HAibQGT FTSDH SKYLD PFYAG LRDLA | 32 |
| | LMGGP SSGAP PPSC | |
| 68 | HAibQGT FTSDM SKYLD GPWAG DFVQW | 33 |
| | LIDGG PSSGA PPPSC | |
| 70 | HAibQGT FTSDQ SKYLD APWAG DFVMW | 34 |
| | LIDGG PSSGA PPPSC | |
| 76 | HAibQGT FTSDD SKYLD KPWAP DFVMW | 35 |
| | LLDGG PSSGA PPPSC | |
| 77 | HAibQGT FTSDK SWYLD GPWAG DFVLW | 36 |
| | LIEGG PSSGA PPPSC | |

*These sequences have an amino acid deletion when aligned
to the sequence of Formula I.

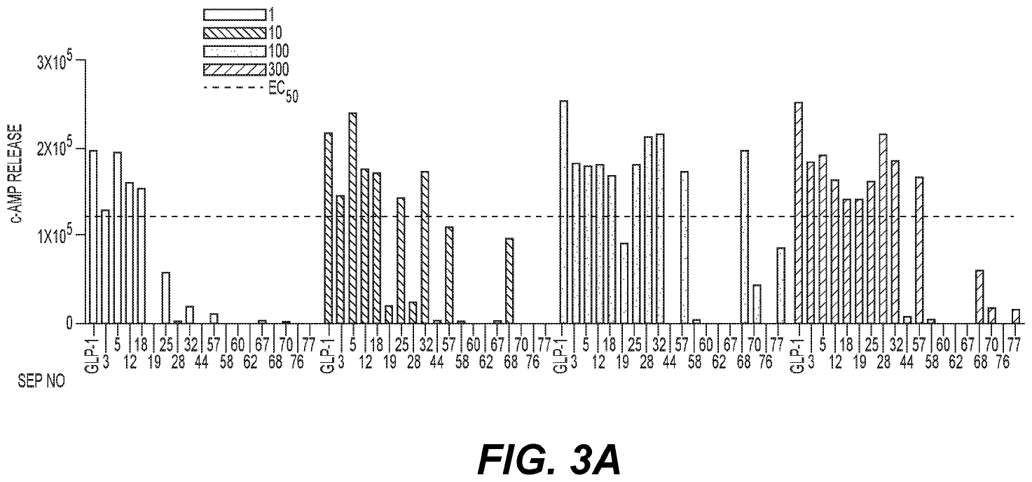

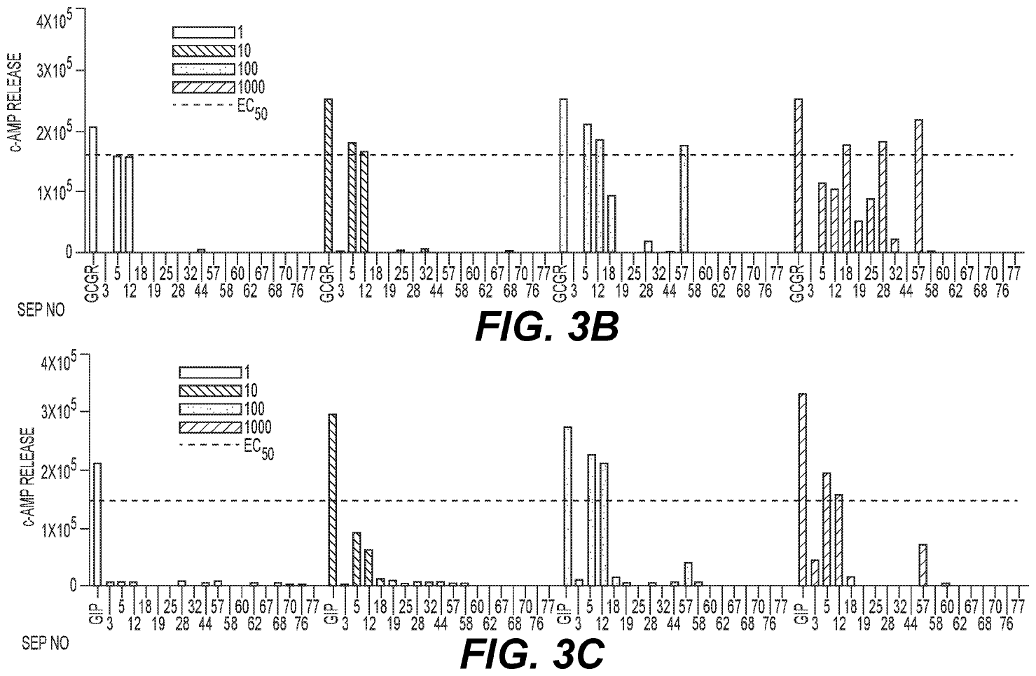

18 individual peptides were again screened using cAMP assay against the three target cell lines GLP-1R+ cells, GCGR+ cells, GIPR+ cells at 4 different concentrations (FIGS. 3A-3C).

### Example 2: Screening for Candidates Suitable for Modifications with Fatty Acids

### Methods

### Lipid Modifications (A Type)

The peptide and C18-γGlu-2OEG-MAL (F12) were dissolved in DMSO containing 0.3% TEA (v/v) solution. Each solution was mixed at a volume ratio of 1:1. The concentration of peptide was 5 mg/mL, and the molar ratio was 1:2 (peptide:lipid). The mixture was reacted at 25° C. for 30 min with gently shaking. Lipidated peptides were purified by Prep-LC and the eluate was collected in individual fractions. The ACN contained in the fractionated solution was evaporated using the centrifugal evaporator at 45° C. for 40 min. The solvent was changed to water by ultrafiltration. The purified samples were analyzed by reversed phase-HPLC for purity check. The samples were lyophilized at −88° C. for 18 hr and then stored at −20° C.

### Lipid and Biotin Modifications (B Type)

The peptide, C18-γGlu-2OEG-MAL (F12), and biotin-N-hydroxysuccinimide ester (B1-NHS, B38) were dissolved in DMSO containing 0.3% TEA (v/v) solution. First, the peptide and F12 were mixed at a volume ratio of 1:1. The concentration of peptide was 5 mg/mL, and the molar ratio was 1:2 (peptide:lipid). The mixture was reacted at 25° C. for 10 min with gently shaking. And then, B38 was added at a volume ratio of 1:0.2 and the molar ratio was 1:3 (peptide: biotin). The mixture was reacted at 25° C. for 60 min with gently shaking. Lipidated and biotinylated peptides were purified by Prep-LC and the eluate was collected in individual fractions. The ACN contained in the fractionated solution was evaporated using the centrifugal evaporator at 45° C. for 40 min. The solvent was changed to water by ultrafiltration. The purified samples were analyzed by reversed phase-HPLC for purity check. The samples were lyophilized at −88° C. for 18 hr and then stored at −20° C.

### Lipid and Biotin Modifications (C Type)

The peptide, biotin-maleimide (B1-MAL, B1) and C18-γGlu-2OEG-NPC (F16) were dissolved in DMSO containing 0.3% TEA (v/v) solution. First, the peptide and B1-MAL were mixed at a volume ratio of 1:1. The concentration of peptide was 5 mg/mL, and the molar ratio was 1:2 (peptide: biotin). The mixture was reacted at 25° C. for 10 min with gently shaking. And then, C18-NPC were added at a volume ratio of 1:0.2 and the molar ratio was 1:2 (peptide:lipid). The mixture was reacted at 25° C. for 90 min with gently shaking. Lipidated and biotinylated peptides were purified by Prep-LC and the eluate was collected in individual fractions. The ACN contained in the fractionated solution was evaporated using the centrifugal evaporator at 45° C. for 40 min. The solvent was changed to water by ultrafiltration. The purified samples were analyzed by reversed phase-HPLC for purity check. The samples were lyophilized at −88° C. for 18 hr and then stored at −20° C.

### Results

Based on the data from cAMP assays of 18 individual peptides, peptide Nos. 5, 12, 18, and 57 were selected for full screening. Peptide Nos. 5, 12, 18, and 57 were screened using cAMP assays to determine their EC₅₀ the three target cell lines GLP-1R+ cells, GCGR+ cells and GIPR+ cells. The relative ratios of EC₅₀ of their respective native ligand over that of the triple agonist peptides are shown in Tables 6-8.

TABLE 6
Percentage change in EC₅₀ relative to GLP-1 at human GLP-1R

| | Percentage change in EC₅₀ |
|---|---|
| | relative to GLP-1 at |
| Peptide No. | human GLP-1R(%) |
| GLP-1 | 100% |
| 5 | 611% |
| 12 | 303% |
| 18 | 40% |
| 57 | 1% |

TABLE 7
Percentage change in EC₅₀ relative to GCG at human GCGR

| | Percentage change in EC₅₀ |
|---|---|
| | relative to GCG at human |
| Peptide No. | GCGR (%) |
| Glucagon | 100% |
| 5 | 725% |
| 12 | 1196% |
| 18 | 1% |
| 57 | 1% |

TABLE 8
Percentage change in EC₅₀ relative to GIP at human GIPR

| | Percentage change in EC₅₀ |
|---|---|
| | relative to GIP at human |
| Peptide No. | GIPR (%) |
| GIP | 100% |
| 5 | 3% |
| 12 | 4% |
| 18 | 0% |
| 57 | 0% |

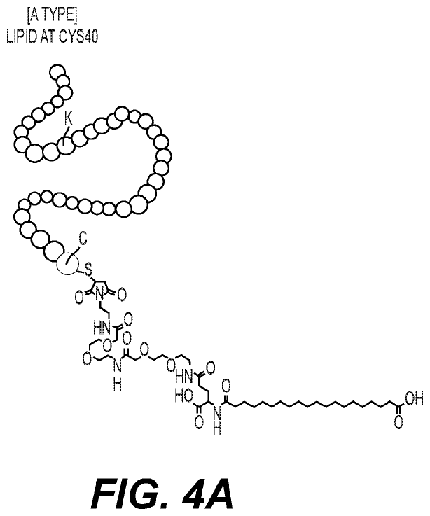

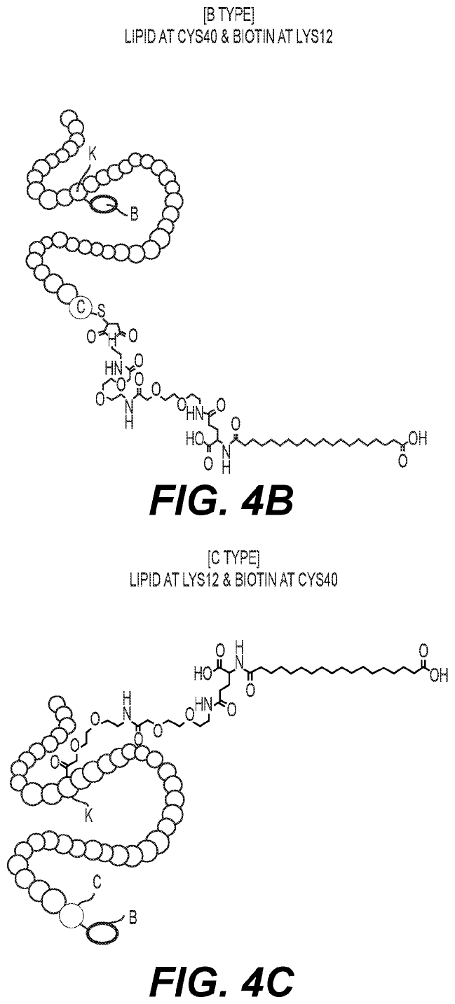

The direct use of native polypeptides as biopharmaceuticals is often limited by their very short systemic half-lives resulting from a rapid metabolism, enzymatic degradation, and, for smaller proteins and peptides, effective renal clearance. Modifications such as biotinylation and lipidation are introduced to improve the stability, bioavailability, and absorption of these peptides in vivo. Exemplary conjugations of a biotin moiety, a fatty acid moiety, or a biotin and fatty acid moiety to triple agonist peptides are shown in FIGS. 4A-4C. For ease of reference, these modifications are termed A type (lipid at Cys40), B type (lipid at Cys40 and biotin at Lys12) and C type (lipid at Lys12 and biotin at Cys40).

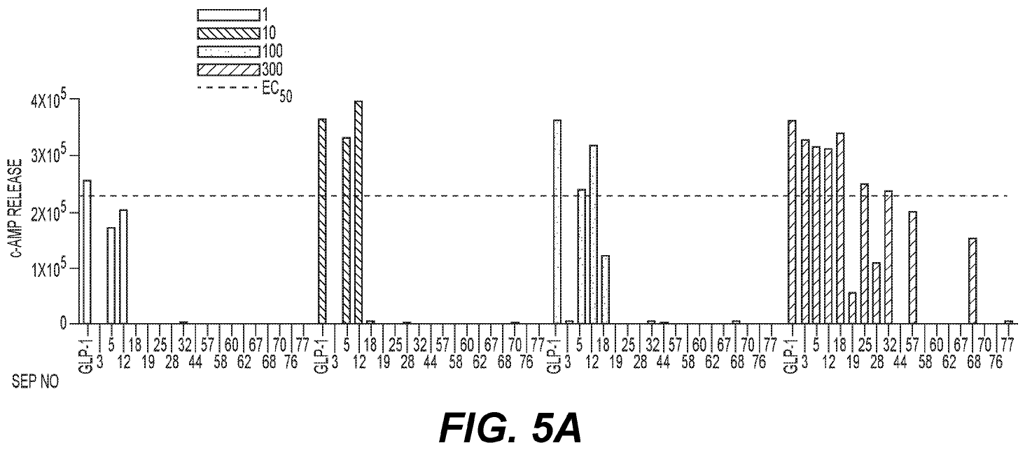

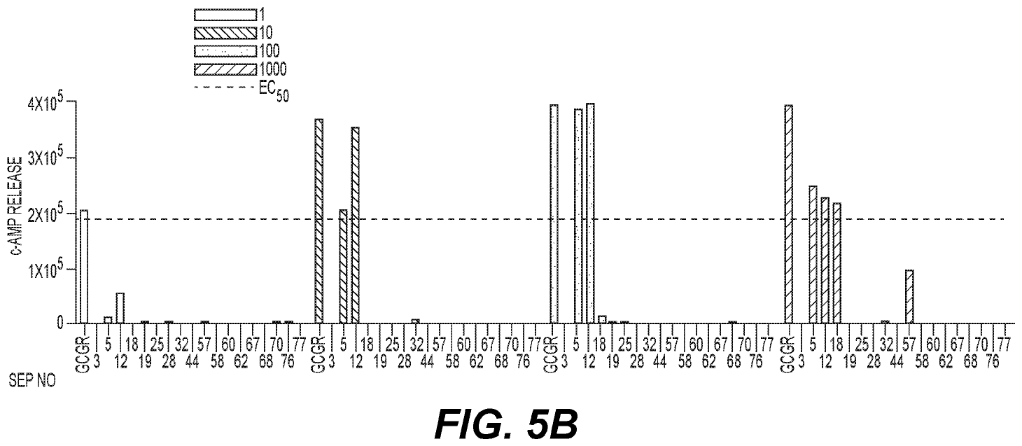

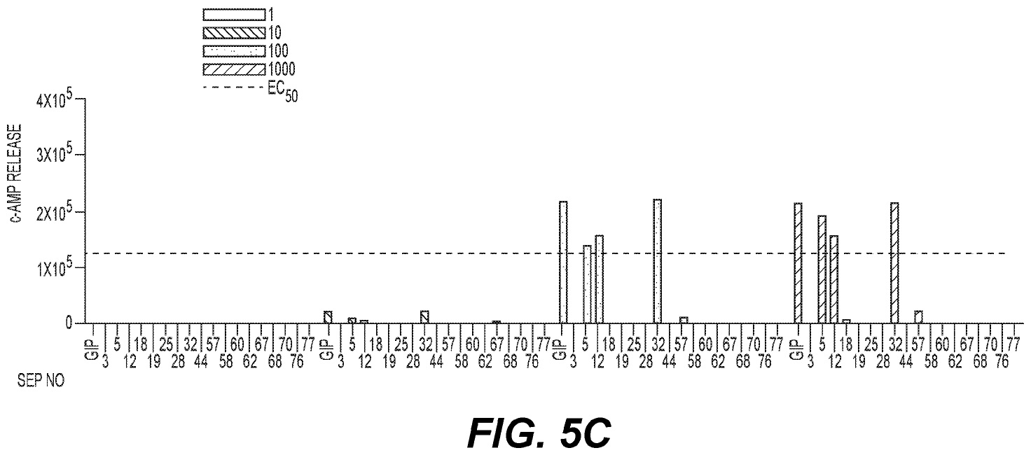

Subsequently, peptides in Table 5 were modified with a lipid molecule conjugated via a cysteine added to the C-terminus of each of the peptides, i.e., A type modification as shown in FIG. 4A. Lipidated version (type A modification) of these peptides were also synthesized to give rise to peptide Nos. 3A, 5A, 12A, 18A, 19A, 25A, 28A, 32A, 44A, 57A, 58A, 60A, 62A, 67A, 68A, 70A, 76A, and 77A. These lipidated peptides were also screened using cAMP assays in three target cell lines GLP-1R+ cells, GCGR+ cells, GIPR+ cells (FIGS. 5A-5C).

Lipidated peptides 5A, 12A, 18A, and 32A were screened using CAMP assays to determine their EC₅₀ the three target cell lines GLP-1R+ cells, GCGR+ cells, GIPR+ cells. The relative ratios of EC₅₀ of their respective native ligand over that of the triple agonist peptides are shown in Tables 9-11.

TABLE 9
Percentage change in EC₅₀ relative to GLP-1 at human GLP-1R

| | Percentage change in EC₅₀ |
|---|---|
| Peptide | relative to GLP-1 at |
| No. | human GLP-1R (%) |
| GLP-1 | 100% |
| 5A | 201% |
| 12A | 296% |
| 18A | 0% |
| 32A | 1% |

TABLE 10
Percentage change in EC₅₀ relative to GCG at human GCGR

| | Percentage change in EC₅₀ |
|---|---|
| Peptide | relative to GCG at human |
| No. | GCGR (%) |
| Glucagon | 100% |
| 5A | 14% |
| 12A | 15% |
| 18A | 0% |
| 32A | 0% |

TABLE 11
Percentage change in EC₅₀ relative to GIP at human GIPR

| | Percentage change in EC₅₀ |
|---|---|
| Peptide | relative to GIP at human |
| No. | GIPR (%) |
| GIP | 100% |
| 5A | 5% |
| 12A | 8% |
| 18A | 0% |
| 32A | 0% |

Based on the CAMP assay results from peptides (5, 12, 18, 57) and lipidated peptides (5A, 12A, 18A, 32A), peptide Nos. 5 and 12 were selected for further study. Lipidation reduced the glucagon and GIP activity.

Peptide No. 5 was further studied with all three types of modifications, A type, B type, and C type (FIGS. 4A-4C). Modified peptides 5A, 5B, and 5C were screened using cAMP assays to determine their EC₅₀ in three target cell lines GLP-1R+ cells, GCGR+ cells, GIPR+ cells. EC₅₀s of peptide No. 5 and its modified forms as well their respective native ligands (STD) are summarized in Table 12. The relative ratios of EC₅₀s of their respective native ligands (STD peptide) over EC₅₀s of each triple agonist peptide are summarized in Table 13. The relative ratios of EC₅₀s of GLP-1 peptide over EC₅₀s of each triple agonist peptide are summarized in Table 14. The data suggest that lipidation at Cys40 reduced the glucagon and GIP activity. C type (Lipidation at Lys 12) show better activity than B type.

TABLE 12
EC₅₀ of peptide No. 5 and its modified forms

| | EC₅₀ (nM) | | | | |
|---|---|---|---|---|---|
| Receptor | STD | 5 | 5A | 5B | 5C |
| GLP-1R | 0.1563 | 0.06051 | 0.1871 | 0.1798 | 0.07725 |
| GCGR | 0.7517 | 0.1556 | 9.286 | 15.1 | 3.229 |
| GIPR | 2.031 | 13.25 | 140 | 97.2 | 24.18 |

TABLE 13
Ratios of EC₅₀ of STD peptide over triple agonist peptides

Activity relative to STD peptide (%)

| | 5 | 5A | 5B | 5C |
|---|---|---|---|---|
| GLP-1 | 258% | 84% | 87% | 202% |
| Glucagon | 483% | 8% | 5% | 23% |
| GIP | 15% | 1% | 2% | 8% |

TABLE 14
Ratios of EC₅₀ of GLP-1 peptide over triple agonist peptides

Activity relative to GLP-1 (%)

| | 5 | 5A | 5B | 5C |
|---|---|---|---|---|
| GLP-1 | 100% | 100% | 100% | 100% |
| Glucagon | 187% | 10% | 6% | 12% |
| GIP | 6% | 2% | 2% | 4% |

### Example 3: In Vivo Efficacy Study for Body Weight Loss in Normal Mice

### Methods

To confirm the therapeutic effect of the peptide in vivo, polypeptides were administered to mice to measure changes in body weight. First, normal C57BL/6 mice were administered with polypeptide by subcutaneous injection at a dose of 20 nmol/kg or 30 nmol/kg at Day 0, 1 and 2 (once daily, QD) or Day 0 and 3 (every other day, Q2D), respectively. The peptides were delivered at 0.02% polysorbate 80 (PS80) in PBS and dosing volume was 10 mL/kg. Body weight was monitored 6 h, QD. Detailed treatment dose and regime are summarized in Table 15.

DD01 peptide is used as a positive control, having the amino acid sequence below with a C-terminal Cysteine that is pegylated (PEG MW 50 kDa):

(SEQ ID NO: 132)
HAibQGT FTSDY SKYLD EQAAK EFVQW LMNTC.

Semaglutide, a GLP-1 receptor agonist from Novo Nordisk, is also used as a positive control.

TABLE 15
Treatment dose and regime.

| Group | Peptide | | Dose | | Dosing |
|---|---|---|---|---|---|
| No. | No. | N | (nmol/kg) | Route | interval |
| 1 | vehicle | 5 | — | SC | QD |
| 2 | 5A | 5 | 20 | SC | QD |
| 3 | 12A | 5 | 20 | SC | QD |
| 4 | 5A | 5 | 30 | SC | Q2D |
| 5 | 12A | 5 | 30 | SC | Q2D |
| 6 | DD01 | 5 | 40 | SC | Q2D |
| 7 | Semaglutide | 5 | 30 | SC | Q2D |

### Results

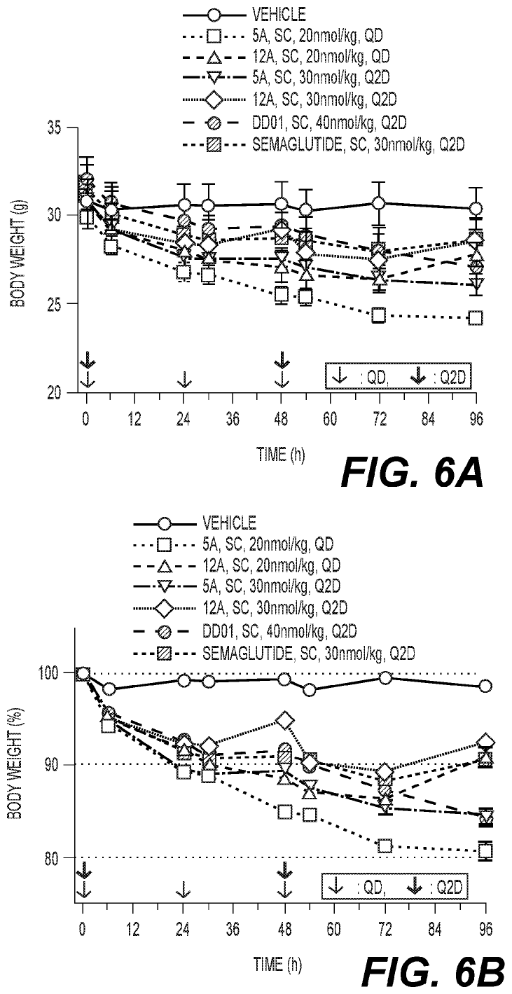

Changes in body weight were measured in all the treatment groups (FIGS. 6A and 6B). Superior body weight loss (BWL) was observed in daily dosing of 5A. 5A showed the similar BWL at a lower dose than DD01. 12A showed similar efficacy to Semaglutide.

### Example 4: Peptide Design and Screen for Increased GIP Activity

### Methods

Peptide Nos. 5 and 12 were used for further peptide design for increasing activity to GIPR. Peptide Nos. 5 and 12, and their derivatives were listed in Table 16. The C-terminal cysteine was introduced to each of these peptides to allow incorporation of modification such as lipidation and/or biotinylation. Lipidated peptides via conjugation to C-terminal cysteine were also synthesized, which are peptide Nos. 5-1A (SEQ ID NO. 50), 5-2A (SEQ ID NO. 51), 5-3A (SEQ ID NO. 52), 5-4A (SEQ ID NO. 53), 5-5A (SEQ ID NO. 54), 5-6A (SEQ ID NO. 55), 12-1A (SEQ ID NO. 56), 12-2A (SEQ ID NO. 57), 12-3A (SEQ ID NO. 58), 12-4A (SEQ ID NO. 59), 12-5A (SEQ ID NO. 60), 12-6A (SEQ ID NO. 61), and 12-7A (SEQ ID NO. 62).

TABLE 16
Peptide sequences derived from peptide Nos. 5

| Peptide | | SEQ |
|---|---|---|
| No. | Amino Acid Sequence | ID NO. |
| 5-1 | YAibQGT FTSDY SKYLD YMMAR DFVQW | 37 |
| | LIEGG PSSGA PPPSC | |
| 5-2 | YAibQGT FTSDY SKYLD YMMQR DFVQW | 38 |
| | LIEGG PSSGA PPPSC | |
| 5-3 | YAibQGT FTSDY SKLLD YMMQR DFVQW | 39 |
| | LLEGG PSSGA PPPSC | |
| 5-4 | HAibQGT FTSDY SKYLD YMMQR DFVQW | 40 |
| | LIEGG PSSGA PPPSC | |
| 5-5 | HAibQGT FTSDY SKLLD YMMQR DFVQW | 41 |
| | LLEGG PSSGA PPPSC | |
| 5-6 | HAibQGT FTSDK SRYLD YMMAR DFVQW | 42 |
| | LIEGG PSSGA PPPSC | |
| 12-1 | YAibQGT FTSDW SKYLD QQMAQ DFVDW | 43 |
| | LINGG PSSGA PPPSC | |
| 12-2 | YAibQGT FTSDW SKYLD QQMQQ DFVDW | 44 |
| | LINGG PSSGA PPPSC | |
| 12-3 | YAibQGT FTSDW SKYLD QQMQQ DFVDW | 45 |
| | LLNGG PSSGA PPPSC | |
| 12-4 | HAibQGT FTSDW SKYLD QQMQQ DFVDW | 46 |
| | LINGG PSSGA PPPSC | |
| 12-5 | HAibQGT FTSDW SKYLD QQMQQ DFVDW | 47 |
| | LLNGG PSSGA PPPSC | |
| 12-6 | HAibQGT FTSDK SRYLD QQMAQ DFVDW | 48 |
| | LINGG PSSGA PPPSC | |
| 12-7 | HAibEGT FTSDW SKYLD QQMAQ DFVDW | 49 |
| | LINGG PSSGA PPPSC | |

### Results

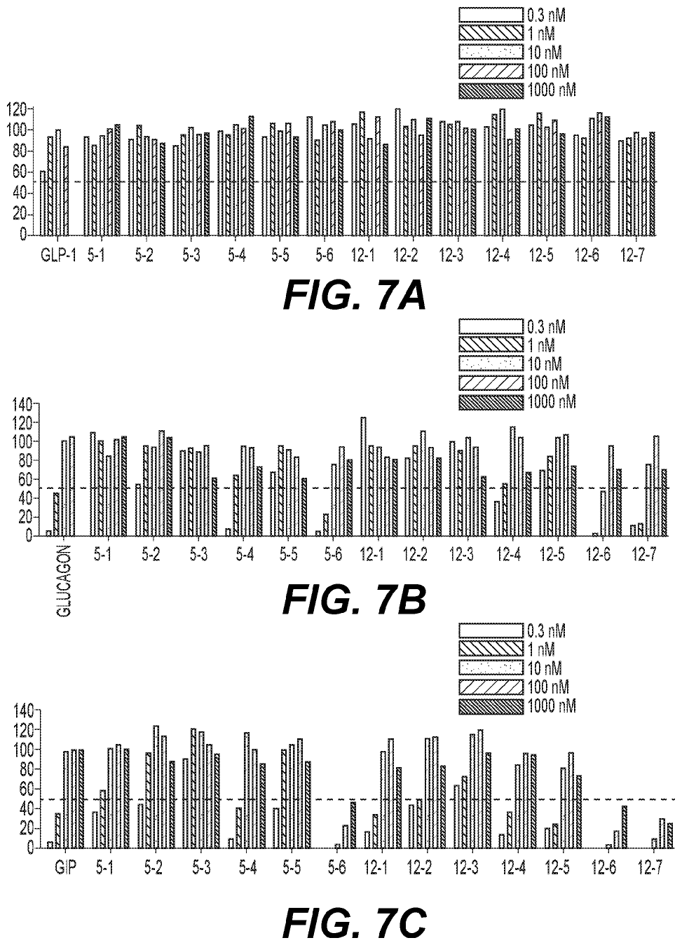

Peptide Nos. 5-1, 5-2, 5-3, 5-4, 5-5, 5-6, 12-1, 12-2, 12-3, 12-4, 12-5, 12-6, and 12-7 were screened using cAMP assays in three target cell lines GLP-1R+ cells, GCGR+ cells, GIPR+ cells (FIGS. 7A-7C).

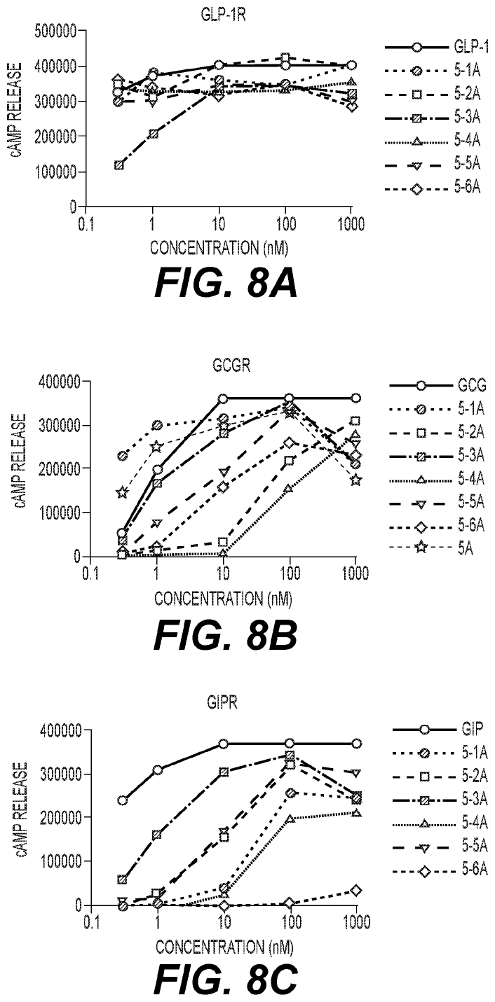

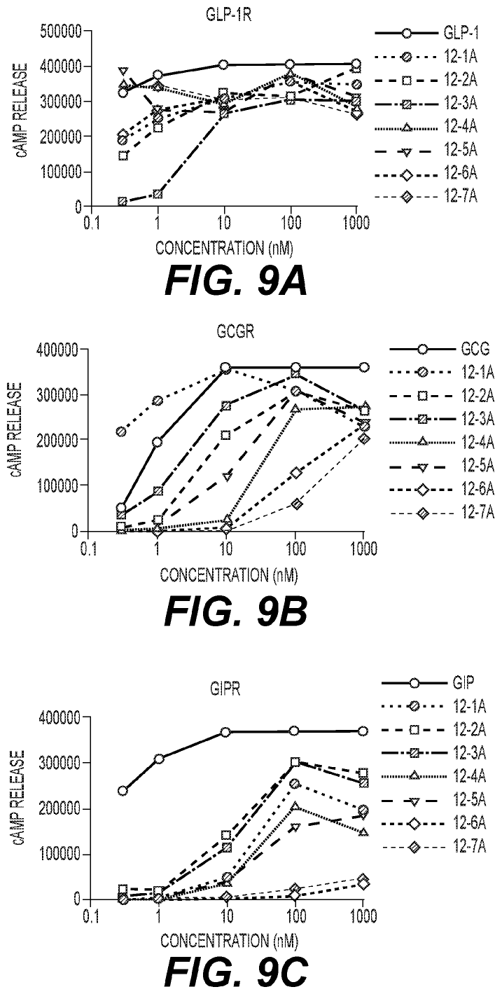

Lipidated version (type A modification) of these peptides were also synthesized to give rise to peptide Nos. 5-1A, 5-2A, 5-3A, 5-4A, 5-5A, 5-6A, 12-1A, 12-2A, 12-3A, 12-4A, 12-5A, 12-6A, and 12-7A (SEQ ID NOs. 50-62). These lipidated peptides were also screened using cAMP assays in three target cell lines GLP-1R+ cells, GCGR+ cells, GIPR+ cells (FIGS. 8A-8C and 9A-9C).

Peptide Nos. 5A, 5-1A, 5-2A, 5-5A were further were screened using CAMP assays to determine their EC₅₀ in three target cell lines GLP-1R+ cells, GCGR+ cells, GIPR+ cells. EC₅₀s of peptide No. 5A, 5-1A, 5-2A, 5-5A as well their respective native ligands (STD) are summarized in Table 17. The relative ratios of EC₅₀s of their respective native ligands (STD peptide) over EC₅₀s of each triple agonist peptide are summarized in Table 18. The relative ratios of EC₅₀s of GLP-1 peptide over EC₅₀s of each triple agonist peptide are summarized in Table 19.

TABLE 17
EC₅₀s of Peptide Nos. 5A, 5-1A, 5-2A, 5-5A

| | EC₅₀ (nM) | | | | |
|---|---|---|---|---|---|
| | STD | 5A | 5-1A | 5-2A | 5-5A |
| GLP-1R | 0.5028 | 0.0967 | 0.269 | 0.3477 | 0.0747 |
| GCGR | 0.1406 | 1.71 | 0.5762 | 22.98 | 9.429 |
| GIPR | 0.7323 | 157.4 | 53.88 | 24.71 | 5.175 |

TABLE 18
Ratios of EC₅₀ of STD peptide over triple agonist peptides

Activity relative to STD peptide (%)

| | 5A | 5-1A | 5-2A | 5-5A |
|---|---|---|---|---|
| GLP-1R | 520% | 187% | 145% | 673% |
| GCGR | 8% | 24% | 1% | 1% |
| GIPR | 0% | 1% | 3% | 14% |

TABLE 19
Ratios of EC₅₀ of GLP-1 peptide over triple agonist peptides

Activity relative to GLP-1 (%)

| | 5A | 5-1A | 5-2A | 5-5A |
|---|---|---|---|---|
| GLP-1R | 100% | 100% | 100% | 100% |
| GCGR | 2% | 13% | 0% | 0% |
| GIPR | 0% | 1% | 2% | 2% |

### Example 5: In Vivo Efficacy Study for Body Weight Loss in Normal Mice

### Methods

To confirm the therapeutic effect of the peptide in obesity, diabetes, or NASH, polypeptides were administered to mice to measure changes in food intake, blood glucose and body weight. Frist, normal C57BL/6 mice were administered with polypeptide by subcutaneous injection at a dose of 20 nmol/kg or 30 nmol/kg at Day 0, 1 and 2 (once daily, QD) or Day 0 and 3 (every other day, Q2D), respectively. In addition, a NASH animal model was prepared by feeding a high fat, high fructose, high cholesterol diet to normal C57BL/6 mice (approximately 6 weeks old) for approximately 21 weeks, and increasing the body weights of the mice to approximately 50 g on average. Thereafter, polypeptides were administered by subcutaneous injection at dose of 20 nmol/kg once every other day for 2 weeks. Body weight and food intake were measured once every other day given points of time. After 2 weeks of treatment period, mice were sacrificed, then liver weight, hepatic triglyceride, and NASH related biomarkers were measured.

The peptides 5A, 5-1A, 5-2A, 5-3A, 5-4A, and 5-5A administered at 20 nmol/kg, s.c. QD as shown in Table 20. DD01 administered at 40 nmol/kg, s.c. Q2D as positive control was delivered at 0.02% PS80 in PBS and dosing volume was 10 mL/kg. Body weight was monitored QD for 4 days.

TABLE 20
Treatment dose and regime.

| Group | Peptide | | Dose | | Dosing |
|---|---|---|---|---|---|
| No. | No. | N | (nmol/kg) | Route | interval |
| 1 | 5A | 5 | 20 | SC | QD |
| 2 | 5-1A | 5 | 20 | SC | QD |
| 3 | 5-2A | 5 | 20 | SC | QD |
| 4 | 5-3A | 5 | 20 | SC | QD |
| 5 | 5-4A | 5 | 20 | SC | QD |
| 6 | 5-5A | 5 | 20 | SC | QD |
| 7 | DD01 | 5 | 40 | SC | Q2D |

Peptides 5A, 12-1A, 12-2A, 12-3A, 12-4A, 12-5A, and 12-7A were administered at 20 nmol/kg, s.c. QD, as summarized in Table 21. The peptides were delivered at 0.02% PS80 in PBS and dosing volume was 10 mL/kg. Food intake, blood glucose, and body weight changes were monitored QD.

TABLE 21
Treatment dose and regime.

| Group | Peptide | | Dose | | Dosing |
|---|---|---|---|---|---|
| No. | No. | N | (nmol/kg) | Route | interval |
| 1 | 5A | 5 | 20 | SC | QD |
| 2 | 12-1A | 5 | 20 | SC | QD |
| 3 | 12-2A | 5 | 20 | SC | QD |
| 4 | 12-3A | 5 | 20 | SC | QD |
| 5 | 12-4A | 5 | 20 | SC | QD |
| 6 | 12-5A | 5 | 20 | SC | QD |
| 7 | 12-7A | 5 | 40 | SC | QD |

### Results

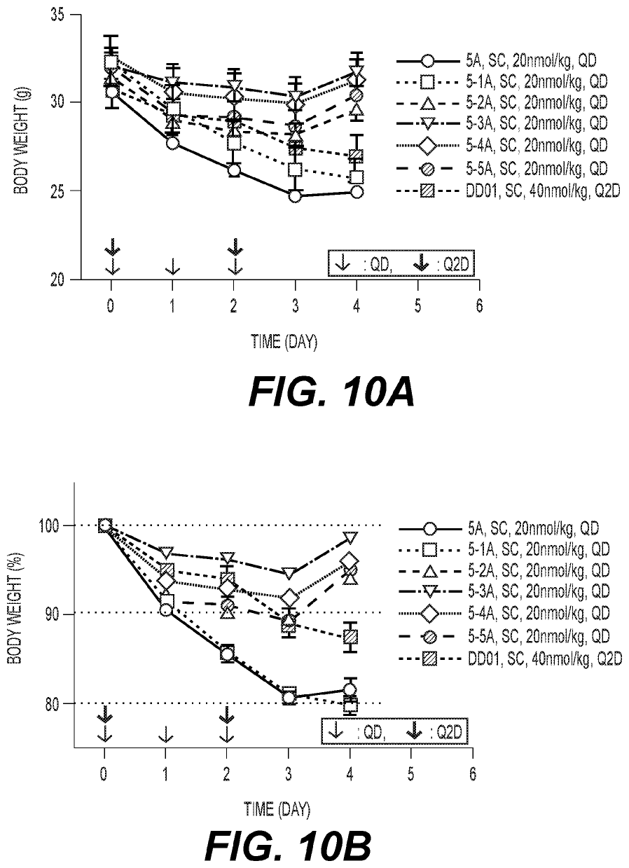

The therapeutic effects of the peptides were assessed in mice. Body weight was monitored over a period of 4 days in mice treated with peptides 5A, 5-1A, 5-2A, 5-3A, 5-4A, and 5-5A (FIGS. 10A-10B). Superior body weight loss effects were observed in 5A and 5-1A treated groups. Based on the body weight measurement, the therapeutic efficacy was most profound in groups treated with peptides 5A and 5-1A, followed by 5-2A and 5-5A, and then 5-4A, with least effect observed in 5-3A.

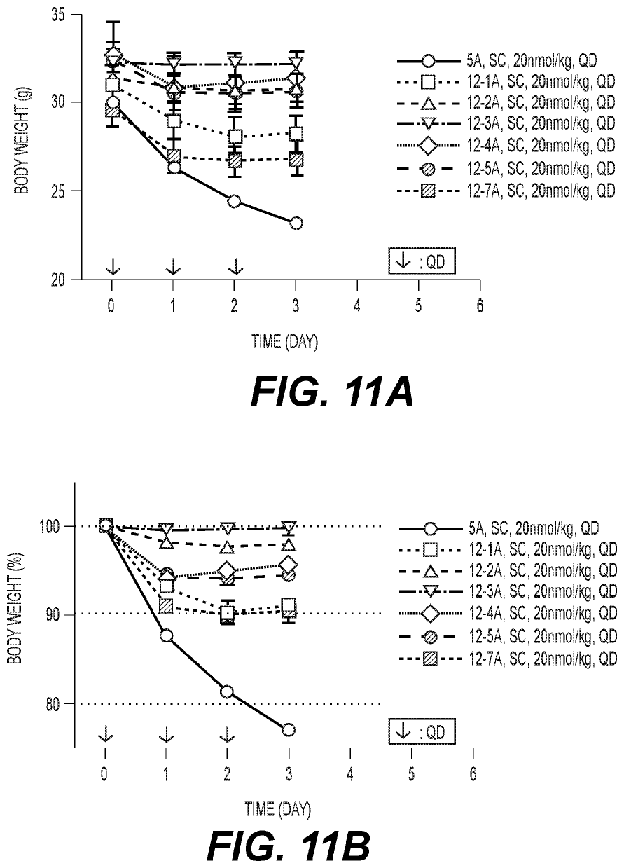

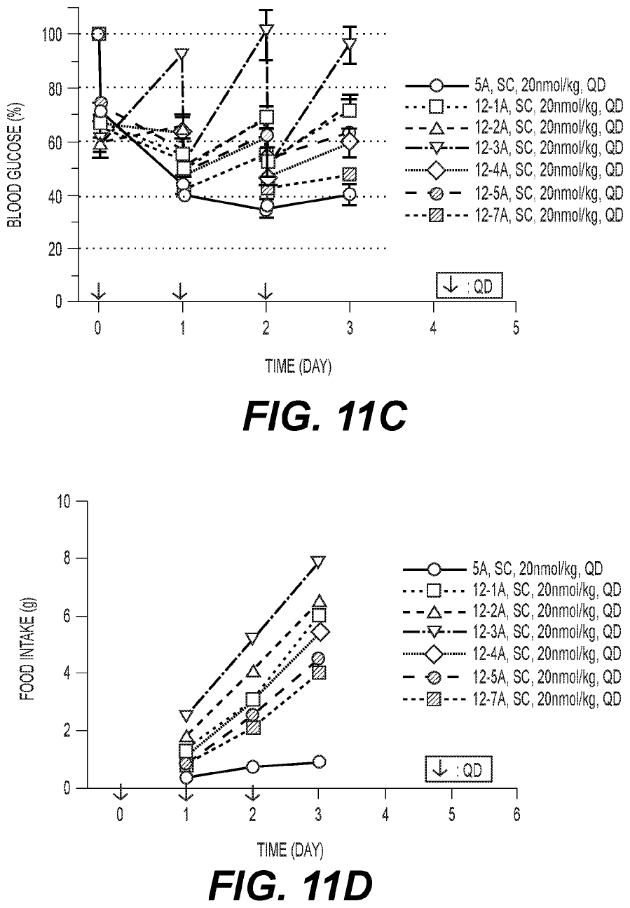

Food intake, blood glucose, and body weight changes were monitored over a period of 3 days in mice treated with peptides 5A, 12-1A, 12-2A, 12-3A, 12-4A, 12-5A, and 12-7A (FIGS. 11A-11D).

### Example 6: Peptide Screening for Improved Sites for Lipidation

### Methods

Peptides 5, 5-1, 5-2, 5-3, 5-4, and 5-5 were synthesized with lipidation at new positions to further improve activities of these peptides when lipidated. Three additional sequences were also synthesized as peptide Nos. 5-6D, 5-7D, and 5-3F. Sequences are in Table 22 with lipidation at indicated residue (K*) without lipidation at Cys40. The fatty acid derivative used for conjugation here is 2OEG-γGlu-C18. Sites and residues suitable for lipidating these peptides are summarized in Table 23.

TABLE 22
Peptide sequences

| Peptide | | SEQ |
|---|---|---|
| No. | Amino Acid Sequence | ID NO. |
| 5D | HAibQGT FTSDY SK*YLD YMMAR DFVQW | 74 |
| | LIEGG PSSGA PPPS | |
| 5-1D | YAibQGT FTSDY SK*YLD YMMAR DFVQW | 75 |
| | LIEGG PSSGA PPPS | |
| 5-2D | YAibQGT FTSDY SK*YLD YMMQR DFVQW | 76 |
| | LIEGG PSSGA PPPS | |
| 5-3D | YAibQGT FTSDY SK*LLD YMMQR DFVQW | 77 |
| | LLEGG PSSGA PPPS | |
| 5-4D | HAibQGT FTSDY SK*YLD YMMQR DFVQW | 78 |
| | LIEGG PSSGA PPPS | |
| 5-5D | HAibQGT FTSDY SK*LLD YMMQR DFVQW | 79 |
| | LLEGG PSSGA PPPS | |
| 5-1E | YAibQGT FTSDK* SKYLD YMMAR DFVQW | 80 |
| | LIEGG PSSGA PPPS | |
| 5-1F | YAibQGT FTSDY SKYLD YK*MAR DFVQW | 81 |
| | LIEGG PSSGA PPPS | |
| 5-6D | YAibQGT FTSDY SK*YLD YMMQR DFVQW | 66 |
| | LLEGG PSSGA PPPS | |
| 5-7D | HAibQGT FTSDY SK*YLD YMMQR DFVQW | 69 |
| | LLEGG PSSGA PPPS | |
| 5-3F | YAibQGT FTSDY SKLLD YK*MQR DFVQW | 73 |
| | LLEGG PSSGA PPPS | |

TABLE 23
Residues and positions suitable for modifications

Modification

| Type | Lipidation | Biotinylation |
|---|---|---|
| A | Cys40 | — |
| B | Cys40 | Lys12 |
| C | Lys12 | Cys40 |
| D | Lys12 | — |
| E | Lys10 | — |
| F | Lys17 | — |

### Results

The second peptide screening was designed to screen for peptides with increased activity towards GIPR using peptide Nos. 5 and 12 as lead sequences, without decreases in their glucagon and GLP-1 activity. The was achieved at peptide level but when lipidated at C-terminus. Thus, the third screen was designed to maintain the potency of those peptide derivatives based on peptide Nos. 5 from 2nd screening but change the position for lipidation for improved activities.

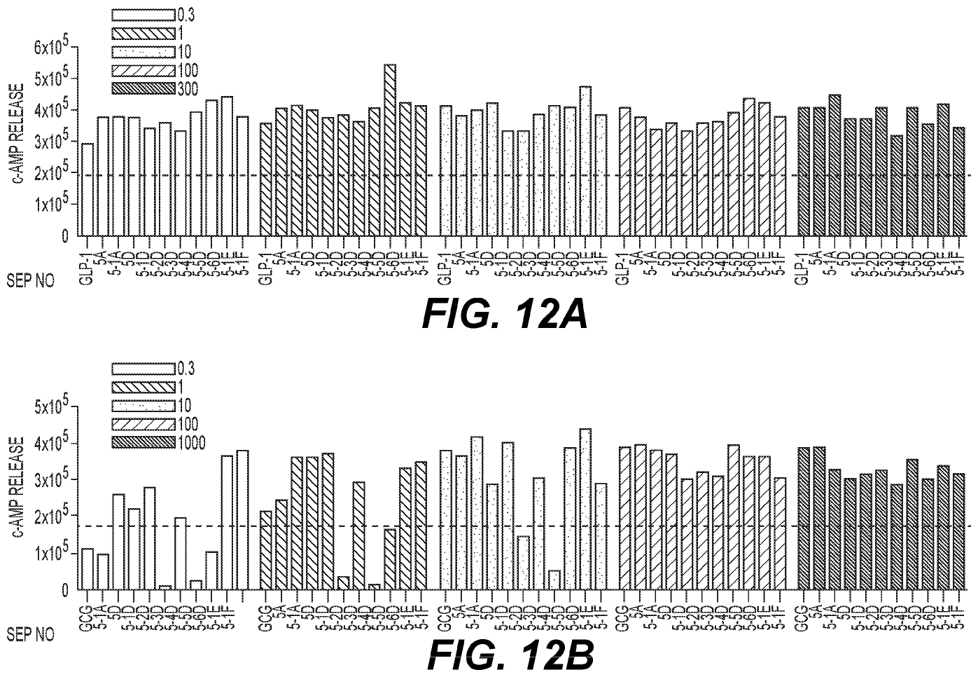

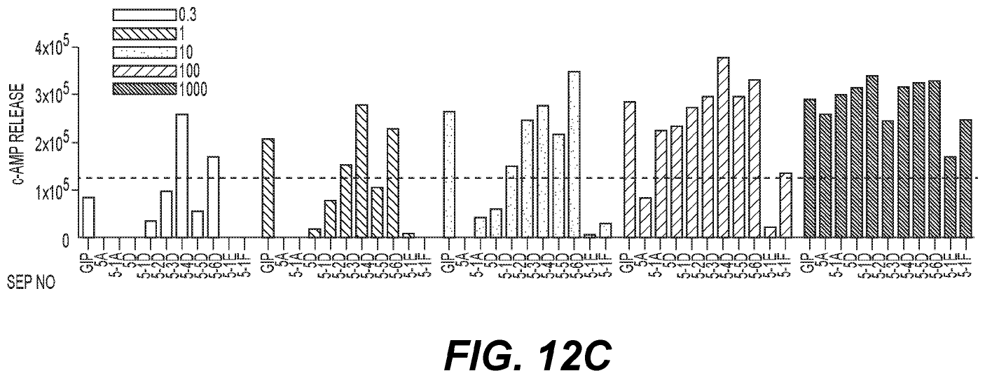

Peptide Nos. 5A, 5-1A, 5D, 5-1D, 5-2D, 5-3D, 5-4D, 5-5D, 5-1E, and 5-1F were screened using cAMP assays in three target cell lines GLP-1R+ cells, GCGR+ cells, GIPR+ cells, at five different concentrations of 0.3 nM, 1 nM, 10 nM, 100 nM, and 300 nM or 1000 nM (FIGS. 12A-12C). The relative ratios of EC₅₀s of their respective native ligands over EC₅₀s of each of the peptides used in the cAMP assays are summarized in Table 24. The relative ratios of EC₅₀s of GLP-1 peptide over EC₅₀s of each of the peptides used in the cAMP assays are summarized in Table 25. The relative ratios of EC₅₀s over EC₅₀s of their respective non-lipidated peptides are summarized in Table 26. Based on the CAMP assays, the activity of these peptides is maintained towards GLP-1R when lipidated at K10, K12, K17, or C40; the activity is maintained towards GCGR when lipidated at K17; and the activity least decreased when lipidated at K12.

TABLE 24
Ratios of EC₅₀ of native ligands over triple agonist peptides

| | Lipidation | relative % to native ligand | | |
|---|---|---|---|---|
| Peptide No. | position | GLP-1R | GCGR | GIPR |
| 5 | — | 611 | 725 | 3 |
| 5A | Cys40 | 201 | 14 | 5 |
| 5D | Lys12 | 35 | 72 | 3 |
| 5-1 | — | 117 | 6654 | 13 |
| 5-1A | Cys40 | 187 | 24 | 1 |
| 5-1D | Lys12 | 151 | 55 | 41 |
| 5-1E | Lys10 | 57 | 74 | 1 |
| 5-1F | Lys17 | 61 | 5030 | 2 |
| 5-2 | — | 495 | 91 | 738 |
| 5-2A | Cys40 | 145 | 1 | 3 |
| 5-2D | Lys12 | 174 | 3 | 93 |
| 5-3 | — | 638 | 969 | 7122 |
| 5-3D | Lys12 | 119 | 11 | 1050 |
| 5-5 | — | 1687 | 260 | 526 |
| 5-5A | Cys40 | 673 | 1 | 14 |
| 5-5D | Lys12 | 305 | 2 | 212 |

TABLE 25
Ratios of EC₅₀ of GLP-1 over triple agonist peptides

| | Lipidation | Ratio vs. GLP-1R | | |
|---|---|---|---|---|
| Peptide No. | position | GLP-1R | GCGR | GIPR |
| 5 | — | 100 | 119 | 0.5 |
| 5A | Cys40 | 100 | 7 | 2 |
| 5D | Lys12 | 100 | 204 | 8 |
| 5-1 | — | 100 | 5665 | 11 |
| 5-1A | Cys40 | 100 | 13 | 1 |
| 5-1D | Lys12 | 100 | 36 | 27 |
| 5-1E | Lys10 | 100 | 130 | 1 |
| 5-1F | Lys17 | 100 | 8247 | 3 |
| 5-2 | — | 100 | 18 | 149 |
| 5-2A | Cys40 | 100 | 0 | 2 |
| 5-2D | Lys12 | 100 | 1 | 54 |
| 5-3 | — | 100 | 152 | 1117 |
| 5-3D | Lys12 | 100 | 9 | 880 |
| 5-5 | — | 100 | 15 | 31 |
| 5-5A | Cys40 | 100 | 0 | 2 |
| 5-5D | Lys12 | 100 | 1 | 70 |

TABLE 26
Ratios of EC₅₀ of candidate triple agonist peptides
over their respective non-lipidated form

| | Lipidation | Relative % to non-lipidated peptide | | |
|---|---|---|---|---|
| Peptide No. | position | GLP-1R | GCGR | GIPR |
| 5 | — | 100 | 100 | 100 |
| 5A | Cys40 | 33 | 2 | 167 |
| 5D | Lys12 | 6 | 10 | 93 |
| 5-1 | — | 100 | 100 | 100 |
| 5-1A | Cys40 | 159 | 0 | 11 |
| 5-1D | Lys12 | 129 | 1 | 323 |
| 5-1E | Lys10 | 48 | 1 | 5 |
| 5-1F | Lys17 | 52 | 76 | 14 |
| 5-2 | — | 100 | 100 | 100 |
| 5-2A | Cys40 | 29 | 1 | 0.4 |
| 5-2D | Lys12 | 35 | 3 | 13 |
| 5-3 | — | 100 | 100 | 100 |
| 5-3D | Lys12 | 19 | 1 | 15 |
| 5-5 | — | 100 | 100 | 100 |
| 5-5A | Cys40 | 40 | 1 | 3 |
| 5-5D | Lys12 | 18 | 1 | 40 |

### Example 7: In Vivo Efficacy Study for Body Weight Loss in Normal Mice

### Methods

Peptides Nos. 5D, 5-1D, 5-2D, 5-3D, 5-4D, 5-5D, 5-1E, and 5-1F were administered at 20 nmol/kg, s.c. QD as shown in Table 27. The peptides were delivered at 0.02% PS80 in PBS and dosing volume was 10 mL/kg. Food intake, blood glucose, and body weight changes were monitored QD.

EL.EX.14, a triple agonist from published PCT application WO2019/125938 was used as a control having the following amino acid sequence:

(SEQ ID NO: 133).
Y-Aib-QGTFTSDYSI-αMeL-LDKK((2-[2-(2-amino-
ethoxy)-ethoxy]-acetyl)-(γGlu)-CO-(CH₂)₁₈-
CO₂H)AQ-Aib-AFIEYLLE-Aib-GPSS-Aib-APPPS-NH2.

TABLE 27
Treatment dose and regime.

| Group | | | Dose | | Dosing |
|---|---|---|---|---|---|
| No. | Peptide No. | N | (nmol/kg) | Route | interval |
| 1 | 5D | 4 | 20 | SC | QD |
| 2 | 5-1D | 4 | 20 | SC | QD |
| 3 | 5-2D | 4 | 20 | SC | QD |
| 4 | 5-3D | 4 | 20 | SC | QD |
| 5 | 5-4D | 4 | 20 | SC | QD |
| 6 | 5-5D | 4 | 20 | SC | QD |
| 7 | 5-1E | 4 | 20 | SC | QD |
| 8 | 5-1F | 4 | 20 | SC | QD |

### Results

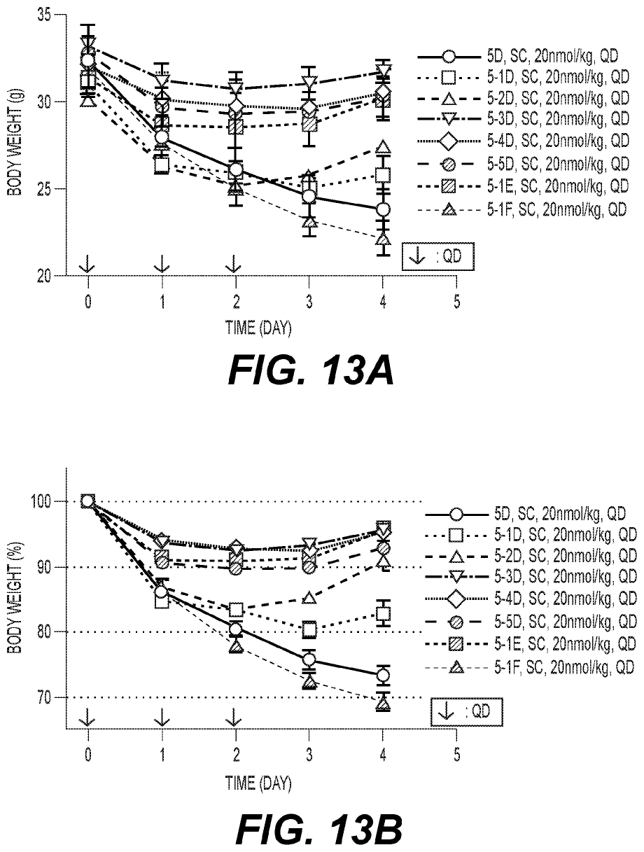

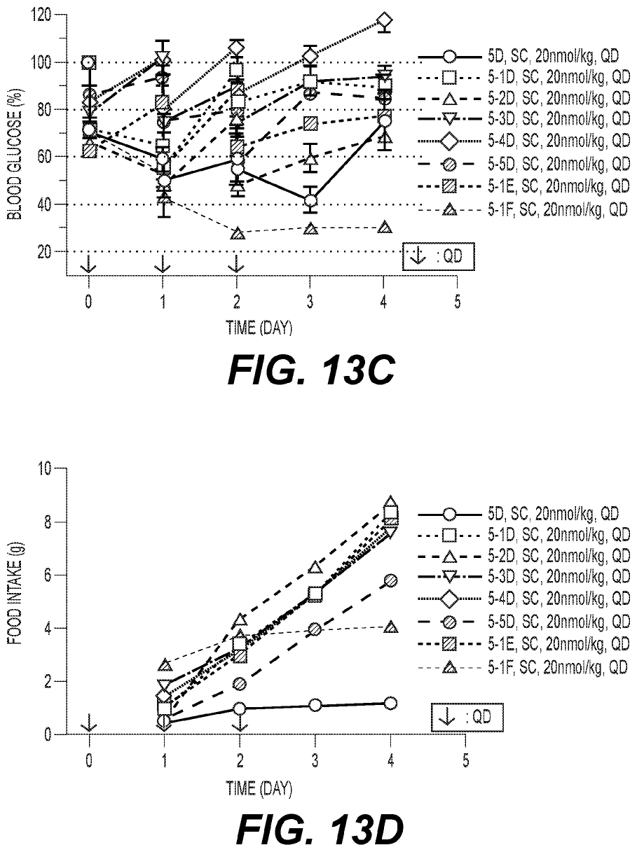

Food intake, blood glucose, and body weight changes were monitored over a period of 4 days in mice treated with peptides 5D, 5-1D, 5-2D, 5-3D, 5-4D, 5-5D, 5-1E, and 5-1F (FIGS. 13A-13D).

Peptides showed significant weight loss effects.

TABLE 28
Body weight loss after 48 or 72 hours in normal mice

| | Dose | Dosing | Weight loss | |
|---|---|---|---|---|
| Peptide No. | (nmol/kg) | interval | (%) | |
| 5A | 10 | QD | 15.7 ± 2.2 | |
| | 20 | QD | 18.8 ± 0.9 | |
| | | | 19.3 ± 1.9 | |
| | | | 22.9 ± 1.5 | |
| | | | 20.1 ± 1.3 | |
| | 30 | Q2D | 14.6 ± 1.8 | |
| 5-1A | 10 | QD | 20.0 ± 2.1 | |
| | 20 | QD | 18.9 ± 2.0 | |
| 5-2A | 20 | QD | 10.4 ± 1.2 | |
| 5-3A | 20 | QD | 5.5 ± 1.6 | |
| 5-4A | 20 | QD | 8.3 ± 0.9 | |
| 5-5A | 20 | QD | 10.6 ± 1.0 | |
| 5C | 10 | QD | 9.6 ± 1.0 | |
| 5C | 20 | QD | 14.1 ± 1.9 | |
| 5D | 20 | QD | 19.4 ± 2.2 | Weight loss |
| 5-1D | 20 | QD | 16.7 ± 1.3 | at 48 hr |
| 5-2D | 20 | QD | 16.6 ± 0.9 | |
| 5-3D | 20 | QD | 7.6 ± 1.3 | |
| 5-4D | 20 | QD | 7.2 ± 0.9 | |
| 5-5D | 20 | QD | 10.3 ± 1.3 | |
| 5-1E | 20 | QD | 9.2 ± 1.8 | |
| 5-1F | 20 | QD | 21.9 ± 2.5 | |
| 12A | 20 | QD | 13.6 ± 1.0 | |
| | 30 | Q2D | 10.5 ± 0.8 | |
| 12-1A | 20 | QD | 9.0 ± 2.2 | |
| 12-2A | 20 | QD | 2.0 ± 0.9 | |
| 12-3A | 20 | QD | 0.2 ± 1.8 | |
| 12-4A | 20 | QD | 4.3 ± 0.9 | |
| 12-5A | 20 | QD | 5.4 ± 0.9 | |
| 12-7A | 20 | QD | 9.5 ± 3.0 | |
| DD01 | 40 | Q2D | 12.7 ± 1.2 | |
| | | | 11.0 ± 3.6 | |
| Semaglutide | 30 | Q2D | 11.6 ± 2.0 | |
| Tirzepatide | 10 | QD | 10.0 ± 0.7 | |
| EL.EX.14 | 10 | QD | 11.0 ± 2.4 | |

### Example 8: In Vivo Efficacy Study in mAMLN Mice Model

### Methods

Peptides 5-1A, 5D, 5-2D, 5-1D, 5-2D, DD01, and EL.EX.14 were administered at 20 nmol/kg, s.c. Q2D for 2 weeks as shown in Table 29. The peptides were delivered at 0.02% PS80 in PBS (only DD01: F1). Food intake and body weight changes were monitored Q2D; blood glucose (0 h, 4 h, 1 d, Q6D, end); serum chemistry (end); hepatic TG (end); inflammation markers (end). Animals were fasted at least 4 hours prior to blood glucose measurement. Control group was administered by vehicle without peptides.

TABLE 29
Treatment dose and regime.

| Group | | | Dose | | Dosing |
|---|---|---|---|---|---|
| No. | Peptide No. | N | (nmol/kg) | Route | interval |
| 1 | Vehicle (CHOW) | 5 | — | SC | Q2D |
| 2 | Vehicle (mAMLN) | 5 | — | SC | Q2D |
| 3 | 5-1A | 5 | 20 | SC | Q2D |
| 4 | 5D | 5 | 20 | SC | Q2D |
| 5 | 5-2D | 5 | 20 | SC | Q2D |
| 6 | 5-1D | 5 | 20 | SC | Q2D |
| 7 | 5-3D | 5 | 20 | SC | Q2D |
| 8 | DD01 | 5 | 20 | SC | Q2D |
| 9 | EL.EX.14 | 5 | 20 | SC | Q2D |

### Results

Non-alcoholic steatohepatitis (NASH) is an obesity-associated liver disease with marked unmet medical need. Various diet-induced obese animal models of NASH have been employed in preclinical research, target discovery and drug development. The trans-fat containing amylin liver NASH (AMLN) diet, high in fat, fructose and cholesterol, has been widely used in ob/ob and C57BL/6J mice for reliably inducing metabolic and liver histopathological changes recapitulating hallmarks of NASH.

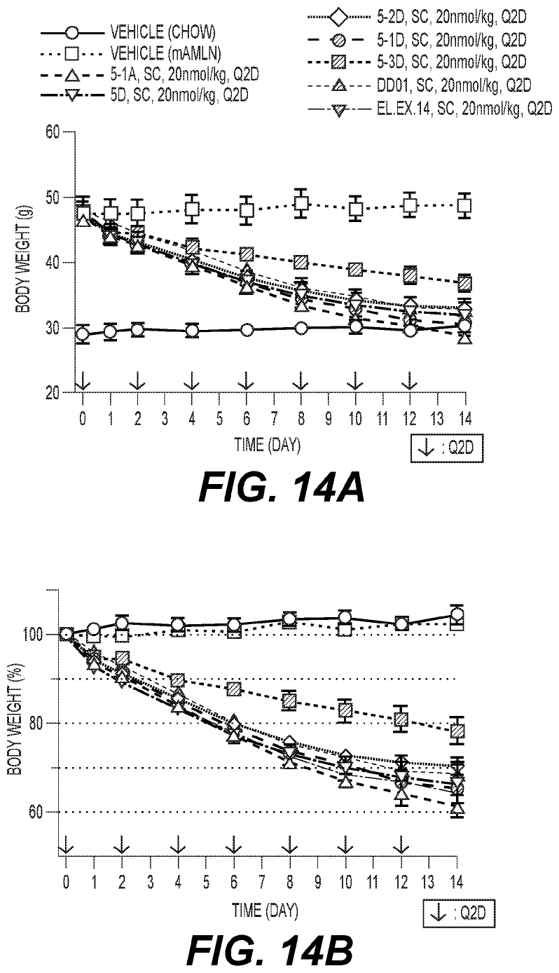

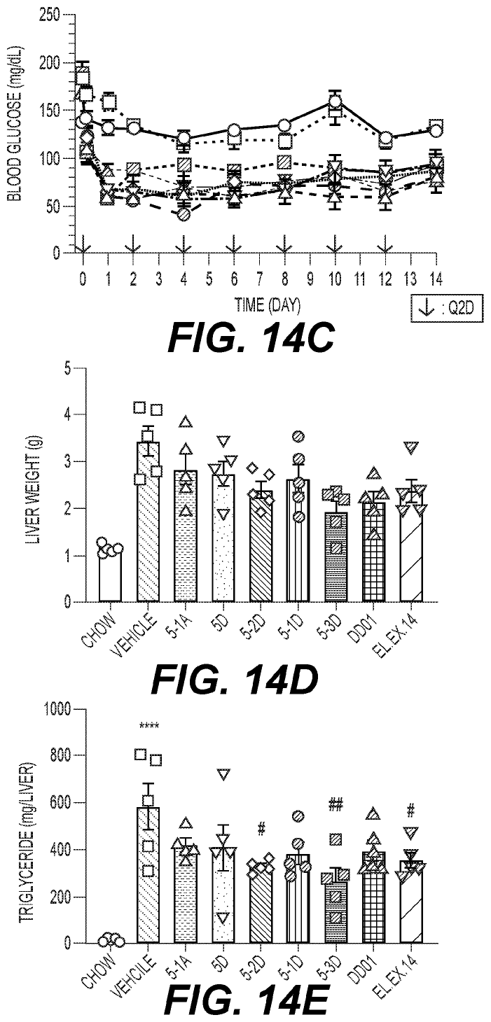

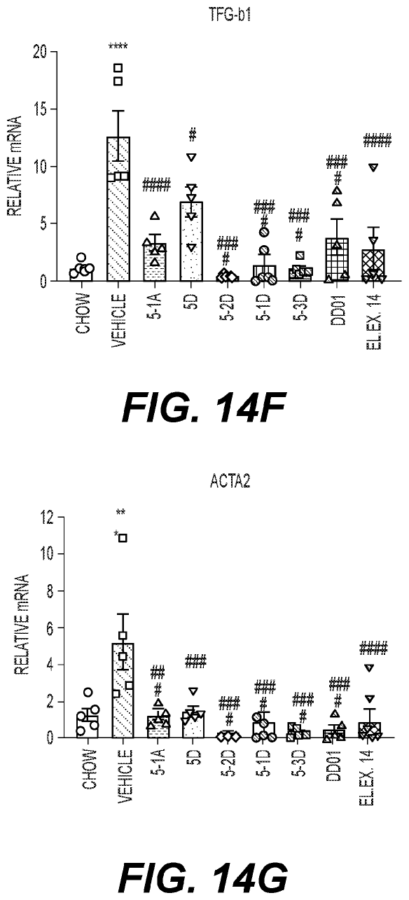

Changes in body weight and in blood glucose in AMLN mice treated with peptides 5-1A, 5D, 5-2D, 5-1D, and 5-3D as well as positive controls DD01 and EL.EX.14 over a period of 2 weeks are shown in FIGS. 14A-14C. All the tested peptides 5-1A, 5D, 5-2D, 5-1D, and 5-3D were able to reduce body weight and reduce blood glucose in the NASH mice.

Liver weight and triglyceride content in the liver at the end of the treatment were determined and shown in FIGS. 14D-14E. All the tested peptides 5-1A, 5D, 5-2D, 5-1D, and 5-3D were able to reduce liver weight and reduce hepatic triglyceride content.

Additionally, expression levels of inflammation marker (TGF-β1) and fibrosis marker (ACTA2, a-SMA) as measured by mRNA levels indicate that the tested peptides 5-1A, 5D, 5-2D, 5-1D, and 5-3D were able to reduce both TGF-β1 and ACTA2 as shown in FIGS. 14F-14G.

### Example 9: Peptide Screening for Improved Sites for Biotinylation

### Methods

The direct use of native polypeptides as biopharmaceuticals is often limited by their very short systemic half-lives resulting from a rapid metabolism, enzymatic degradation, and, for smaller proteins and peptides, effective renal clearance. Modifications such as biotinylation and lipidation are introduced to improve the stability, bioavailability, and absorption of these peptides in vivo. Peptides derived from SEQ ID NOs. 66 and 77 were synthesized with substitution at several amino acids and biotinylation at five different positions to enhance the stability and oral absorption of these peptides. Sequences are in Table 30 with lipidation at indicated Lysine residue (K*), biotinylation at indicated Lysine residue (B*) or Cysteine residue (B^{#}) and substitution of methionine with methoxinine (m*). This modification with methoxinine is to enhance the stability against oxidation. The fatty acid derivative used for conjugation here is OEG2-γGlu-C18. The biotin derivative used for conjugation here is biotin monomer.

In vitro activity study was performed to determine the biotinylation site by conforming the activity change for each receptor according to the biotin conjugation position. The peptides were tested in vitro for activity using cAMP assays on GLP-1R, GCGR and GIPR as previously described. Their signals from cAMP production were compared to native ligand peptide (GLP-1, GCG or GIP).

TABLE 30
Peptide sequences optimized biotinylation site.

| SEQ | |
|---|---|
| ID NO. | Amino Acid Sequence |
| 82 | HAibQGT FTSDY SB*YLD YMMAR DFVQW LIEGG |
| | PSSGA PPPSC* |
| 83 | HAibQGT FTSDY SK*YLD YMMAR DFVQW LIEGG |
| | PSSGA PPPSB# |
| 84 | YAibQGT FTSDY SK*LLD YMMQAib DFVQW |
| | LLEGG PSSGA PPPS |
| 85 | YAibQGT FTSDY SK*LLD YMMQAib DFVQY |
| | LLEGG PSSGA PPPS |
| 86 | YAibQGT FTSDY SK*LLD B*MMQR DFVQW |
| | LLEGG PSSGA PPPS |
| 87 | YAibQGT FTSDY SK*LLD YB*MQR DFVQW |
| | LLEGG PSSGA PPPS |
| 88 | YAibQGT FTSDY SK*LLD YMMQB* DFVQW |
| | LLEGG PSSGA PPPS |
| 89 | YAibQGT FTSDY SK*LLD YMMQR DFVB*W |
| | LLEGG PSSGA PPPS |
| 90 | YAibQGT FTSDY SK*LLD YMMQR DFVQW LLEGG |
| | PSSGA PPPSB* |
| 91 | YAibQGT FTSDY SK*LLD YB*MQAib DFVQW |
| | LLEGG PSSGA PPPS |
| 92 | YAibQGT FTSDY SK*LLD YMMQAib DFVQY |
| | LLEGG PSSGA PPPSB* |
| 93 | YAibQGT FTSDY SK*LLD YYAQR DFVQW LLEGG |
| | PSSGA PPPS |
| 94 | YAibQGT FTSDY SK*LLD YYIQR DFVQW LLEGG |
| | PSSGA PPPS |
| 95 | YAibQGT FTSDY SK*LLD QQAQR DFVQW LLEGG |
| | PSSGA PPPS |
| 96 | YAibQGT FTSDY SK*LLD Ym*m*QR DFVQW |
| | LLEGG PSSGA PPPS |
| 97 | YAibQGT FTSDY SK*YLD YMMQR DFVB*W |
| | LLEGG PSSGA PPPS |
| 98 | YAibQGT FTSDY SK*YLD YMMQR DFVQW |
| | LLEGG PSSGA PPPSB* |

### Results

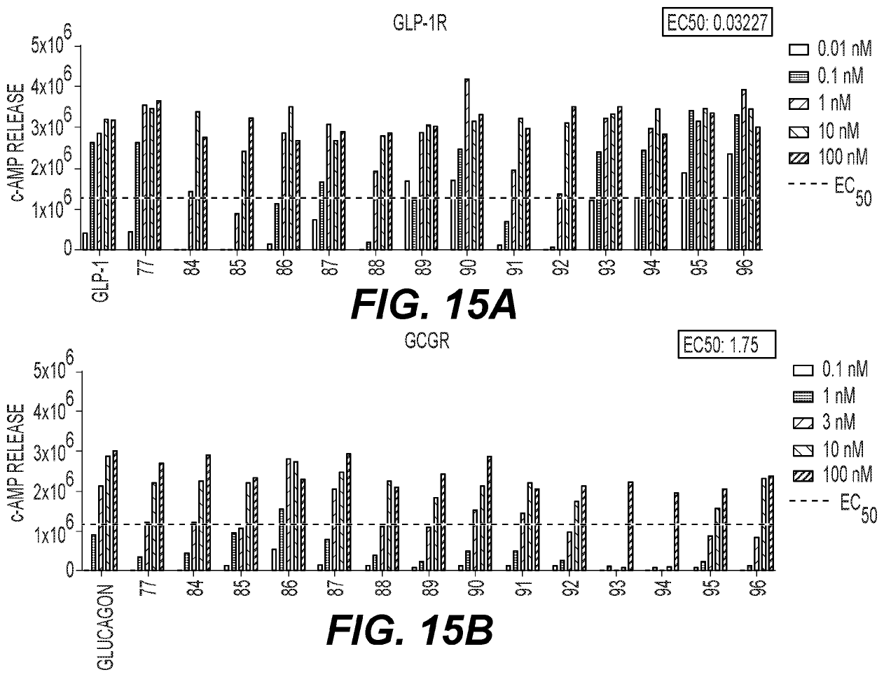

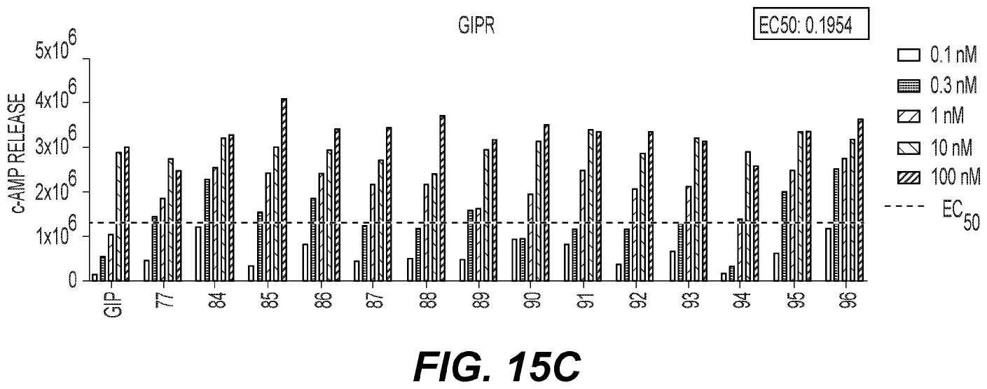

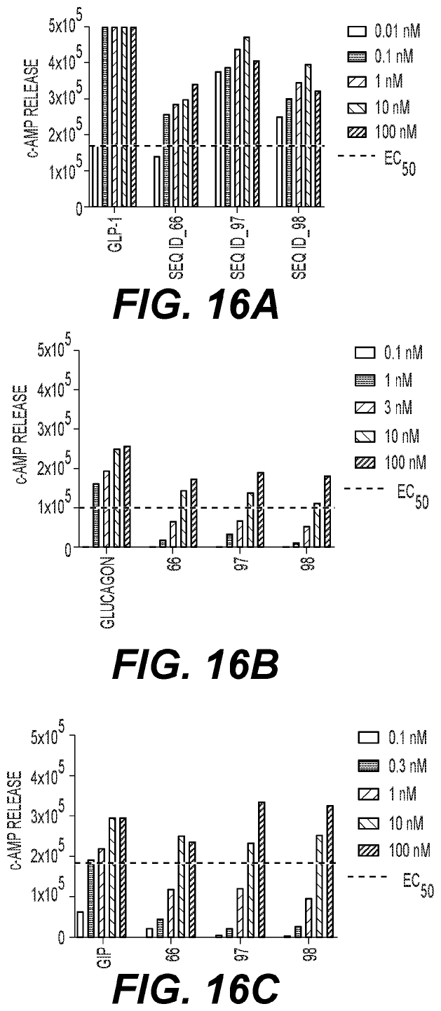

SEQ ID NOs. 77, 84, 85, 86, 87, 88, 89, 90, 91, 92, 93, 94, 95, and 96 were screened using cAMP assay in three target cell lines, GLP-1R+ cells, GCGR+ cells, GIPR+ cells, at five different concentrations of 0.01 nM, 0.1 nM, 1 nM, 10 nM, and 100 nM (FIGS. 15A-15C). SEQ ID NOs. 66, 97, and 98 were screened using cAMP assay in three target cell lines, GLP-1R+ cells, GCGR+ cells, GIPR+ cells, at five different concentrations of 0.1 nM, 0.3 nM, 1 nM, 10 nM, and 100 nM (FIGS. 16A-16C).

SEQ ID NOs. 84, 85, 86, 87, 88, 89, 90, 91, 92, 95, and 96 were selected for full screening and screened using cAMP assay to determine their EC₅₀s the three target cell lines, GLP-1R+ cells, GCGR+ cells, GIPR+ cells. The relative ratios of EC₅₀ of their respective native ligand and GLP-1 over triple agonist peptides are shown in Table 31.

As a result of comparing their activities by biotinylation to SEQ ID NO. 77 at five different positions, biotinylation site that can minimize the loss on peptide activity was determined. The biotinylation site was confirmed again using additional peptide, SEQ ID NOs. 66. Based on these results in cAMP assay, the activity is maintained towards three target receptors when biotinylated at K16, K17, K24, or K40.

TABLE 31
Ratios of EC₅₀ of native ligands and
GLP-1 over triple agonist peptides.

| SEQ ID | Relative % to native ligand | | | Ratio vs. GLP-1R | | |
|---|---|---|---|---|---|---|
| NO. | GLP-1R | GCGR | GIPR | GLP-1R | GCGR | GIPR |
| 84 | 71 | 20 | 335 | 100 | 28 | 470 |
| 85 | 12 | 38 | 143 | 100 | 305 | 1150 |
| 86 | 90 | 142 | 575 | 100 | 157 | 639 |
| 87 | 110 | 66 | 308 | 100 | 60 | 281 |
| 88 | 16 | 20 | 71 | 100 | 120 | 432 |
| 89 | 38 | 45 | 154 | 100 | 117 | 399 |
| 90 | 50 | 19 | 154 | 100 | 39 | 311 |
| 91 | 19 | 26 | 184 | 100 | 135 | 970 |
| 92 | 8 | 19 | 120 | 100 | 243 | 1526 |
| 95 | 1632 | 14 | 316 | 100 | 1 | 19 |
| 96 | 181 | 17 | 102 | 100 | 10 | 56 |
| 97 | 106 | 30 | 123 | 100 | 28 | 117 |
| 98 | 61 | 17 | 205 | 100 | 28 | 338 |

### Example 10: In Vivo Efficacy Study for Body Weight Loss in HFD Mice

### Methods

This study was conducted to confirm the body weight loss effect and select the peptides to be evaluated in high fat diet (HFD) treated mouse, which is widely used as experimental animal model in obesity and diabetes studies. SEQ ID NOs. 86, 87, 89, and 90 were administrated at 20 nmol/kg, s.c. Q2D for 2 weeks as shown in Table 32. The peptides were delivered at 0.02% PS80 in PBS and dosing volume was 10 mL/kg. Body weight changes, blood glucose, and food intake were monitored Q2D. Animals were fasted at least 4 hours prior to blood glucose measurement.

TABLE 32
Treatment dose and regime.

| Group | SEQ ID | | Dose | | Dosing |
|---|---|---|---|---|---|
| No. | NO. | N | (nmol/kg) | Route | interval |
| 1 | 77 | 5 | 20 | SC | Q2D |
| 2 | 86 | 5 | 20 | SC | Q2D |
| 3 | 87 | 5 | 20 | SC | Q2D |
| 4 | 89 | 5 | 20 | SC | Q2D |
| 5 | 90 | 5 | 20 | SC | Q2D |

### Results

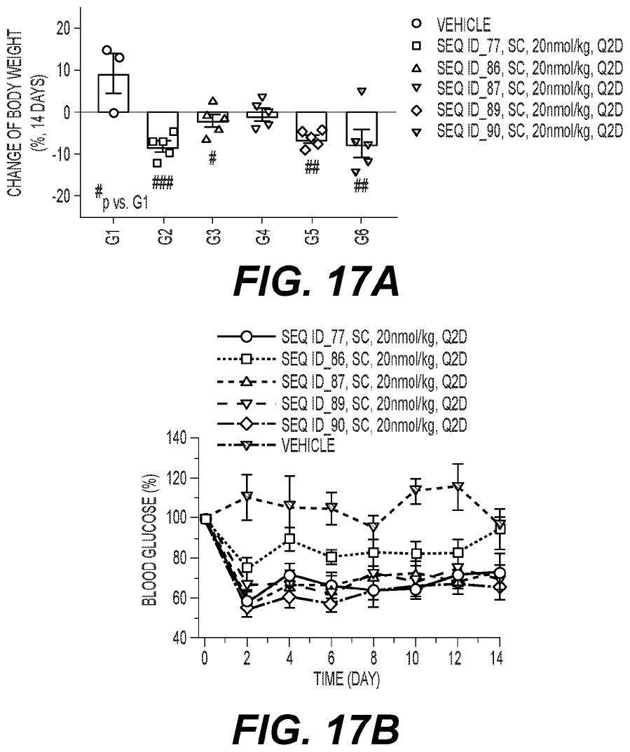

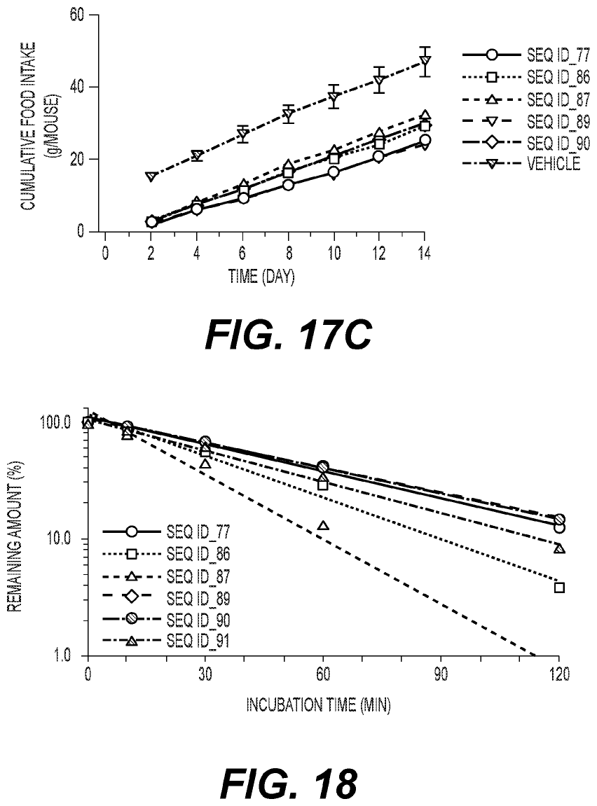

Body weight changes, blood glucose, and food intake were monitored over a period of 2 weeks in HFD mice treated with SEQ ID NOs. 77, 86, 87, 89, and 90 (FIGS. 17A and 17B).

Body weight loss of SEQ ID NOs. 86, 87, 89, and 90, which were biotinylated at different site on SEQ ID NO. 77, was compared to non-biotinylated SEQ ID NO. 77. SEQ ID NOs. 86 and 87 were inferior in body weight loss effect and SEQ ID NOs. 89 and 90 were comparable to SEQ ID NO. 77.

### Example 11: In Vitro Stability Study Against FaSSIF/P

### Methods

This study was conducted to determine the enzymatic stability of SEQ ID NOs. 77, 86, 87, 89, 90, and 91 after biotinylation in artificial intestinal environment. The test samples were prepared by mixing stock solutions of peptide and pancreatin dissolved in FaSSIF solution (pH 6.5) in a weight ratio of 25:1. The test samples were incubated at 37° C. for 120 min. The sampling from test samples was proceeded at the predetermined time points (10, 30, 60, and 120 min) and stopped by 10% TFA solution.

Analytical HPLC was performed to determine the remaining amount on an ACQUITY Premier system (Waters, USA) with an ACQUITY CSH C18 column (Waters, USA) at 35° C. using 0.1% TFA in water and acetonitrile. The remaining amount was determined as a percentage of the peak area at the sampling time point relative to the peak area of the initial sample.

### Results

The enzymatic stability of SEQ ID NOs. 86, 87, 89, 90, and 91 was determined in artificial intestinal environment compared with SEQ ID NO. 77. The half-lives determined by remaining amount in FaSSIF/P are summarized in Table 33. As a result of this study, SEQ ID NOs. 89 and 90 showed intestinal stability not inferior to that of SEQ ID No. 77 (FIG. 18).

TABLE 33
In vitro stability study of SEQ ID NOs. 77,
86, 87, 89, 90, and 91 against FaSSIF/P.

| SEQ ID NO. | Half-life (min) |
|---|---|
| 77 | 39.4 |
| 86 | 25.4 |
| 87 | 16.4 |
| 89 | 43.0 |
| 90 | 42.1 |
| 91 | 33.3 |

### Example 12: In Vivo Efficacy Study in CDA-HFD Mice

### Methods

This study was conducted to evaluate the efficacy of SEQ ID NOs. 77, 87, 89, 90, 95, and 96 to determine the effect of biotinylation and amino acid substitution on efficacy in choline-deficient, L-amino acid-defined, high-fat diet (CDA-HFD) treated mice, which are widely used as experimental animal model in NAFLD/NASH studies. SEQ ID NOs. 77, 87, 89, 90, 95, and 96 were administered at 20 nmol/kg, s.c. Q2D for 4 weeks as shown in Table 34. The peptides were delivered at 0.02% PS80 in PBS and dosing volume was 10 mL/kg. Control groups were administered by vehicle without peptide.

Body weight changes were monitored Q2D. After 4 weeks of treatment period, CDA-HFD mice were sacrificed, then, serum chemistry analysis was conducted with HITACHI 7180 (Hitachi High-Tech Korea, Seongnam, Gyeonggi, Korea) in Genia (Seongnam, Gyeonggi, Korea). ALT and AST for hepatocellular and several lipid markers in serum (LDL and TG) were analyzed.

TABLE 34
Treatment dose and regime.

| | | | Dose | | Dosing |
|---|---|---|---|---|---|
| Group No. | SEQ ID NO. | N | (nmol/kg) | Route | interval |
| 1 | Vehicle (CHOW) | 3 | — | SC | Q2D |
| 2 | Vehicle (CDA-HFD) | 6 | — | SC | Q2D |
| 3 | 77 | 6 | 20 | SC | Q2D |
| 4 | 87 | 6 | 20 | SC | Q2D |
| 5 | 89 | 6 | 20 | SC | Q2D |
| 6 | 90 | 6 | 20 | SC | Q2D |
| 7 | 95 | 6 | 20 | SC | Q2D |
| 8 | 96 | 5 | 20 | SC | Q2D |

### Results

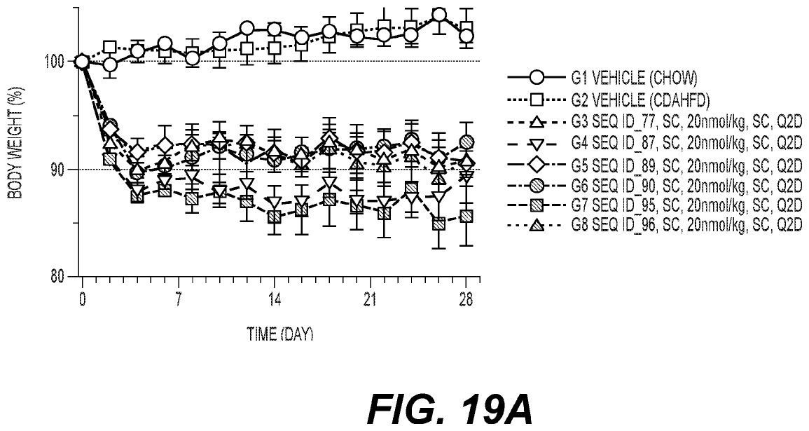

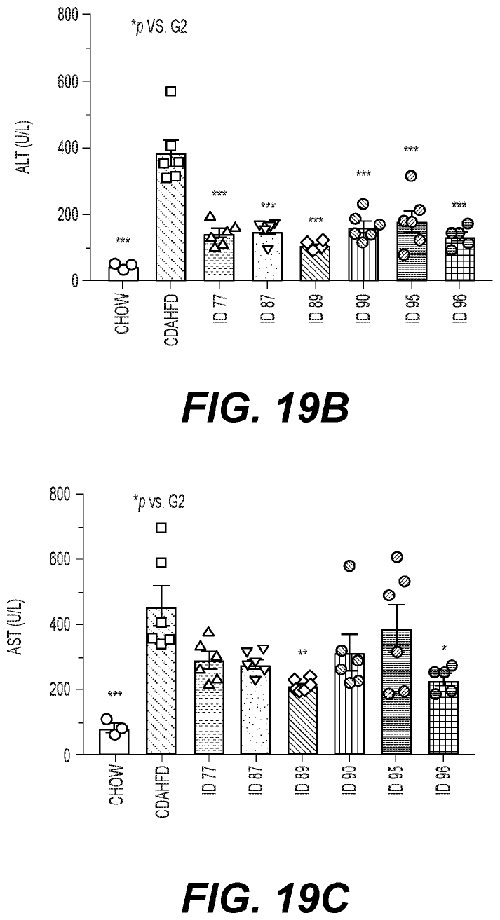

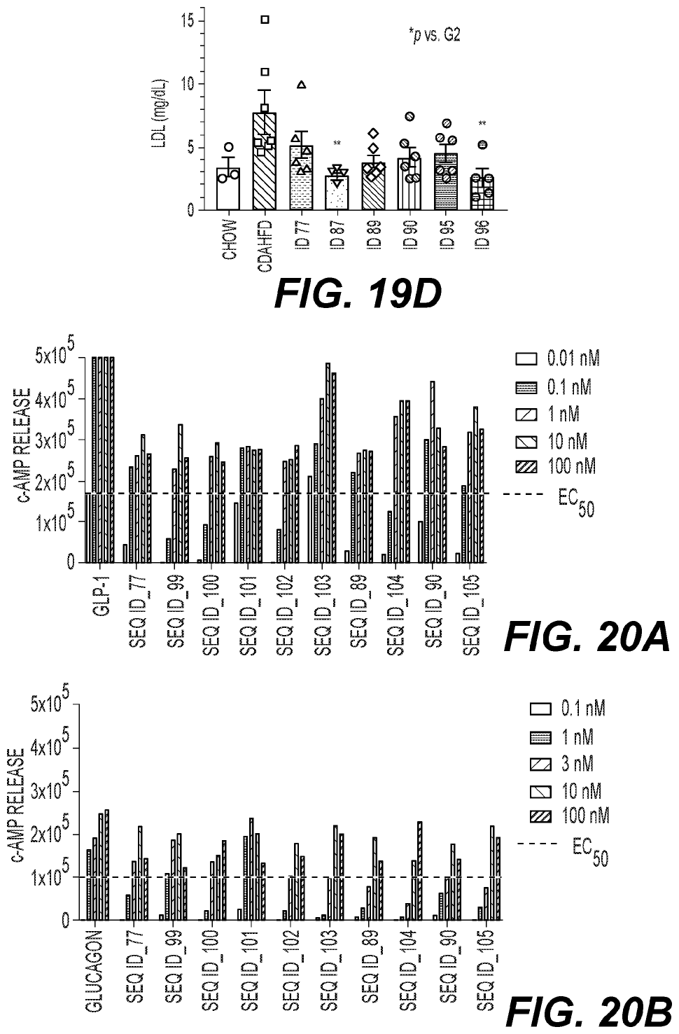

Body weight changes in CDA-HFD mice treated with SEQ ID NOs. 77, 87, 89, 90, 95, and 96 over a period of 4 weeks are shown in FIG. 19A. All the tested peptides were able to reduce body weight loss in the NASH mice model.

At the end of treatment, ALT, AST and LDL for hepatocellular and several lipid markers in serum were analyzed and shown in FIGS. 19B-19D. All test peptides were shown to improve the serum lipid profiles.

### Example 13: Peptide Screening for Improved Stability by Substitution of 20^{th} Amino Acid and Optimization of Lipid Structure

### Methods

It is necessary to optimize the peptide sequence for the oral delivery study by changing the amino acid vulnerable to intestinal enzymes. In addition, the prolonged plasma half-life helps to reduce variabilities on systemic exposure at steady state when administered orally.

Peptides derived from SEQ ID NO. 77 were synthesized with the substitution of 20th amino acid, arginine, to increase the intestinal stability and the optimization of lipid structure to extend in vivo half-life. Sequences are in Table 35 with lipidation at indicated lysine residue (K*, K** or K***), biotinylation at indicated lysine residue (B*) and α-methyl-arginine (αR). The fatty acid derivatives used for conjugation here are 2OEG-γGlu-C18 (K*), 2OEG-γGlu-C20 (K**) or OEG-γGlu-C20 (K***). The biotin derivative used for conjugation here is biotin monomer.

In vitro activity study was performed to determine the substitution of amino acid and lipid structure by conforming the activity change for each receptor. The peptides were tested in vitro for activity using cAMP assays on GLP-1R, GCGR, and GIPR as previously described. Their signals from CAMP production were compared to native ligand peptide (GLP-1, GCG or GIP).

TABLE 35
Peptide sequence

| SEQ | |
|---|---|
| ID NO. | Amino Acid Sequence |
| 99 | YAibQGT FTSDY SK*LLD YMMQH DFVQW |
| | LLEGG PSSGA PPPS |
| 100 | YAibQGT FTSDY SK*LLD YMMQQ DFVQW |
| | LLEGG PSSGA PPPS |
| 101 | YAibQGT FTSDY SK*LLD YMMQαR DFVQW |
| | LLEGG PSSGA PPPS |
| 102 | YAibQGT FTSDY SK***LLD YMMQR DFVQW |
| | LLEGG PSSGA PPPS |
| 103 | YAibQGT FTSDY SK**LLD YMMQR DFVQW |
| | LLEGG PSSGA PPPS |
| 104 | YAibQGT FTSDY SK**LLD YMMQR DFVB*W |
| | LLEGG PSSGA PPPS |
| 105 | YAibQGT FTSDY SK**LLD YMMQR DFVQW |
| | LLEGG PSSGA PPPSB* |

### Results

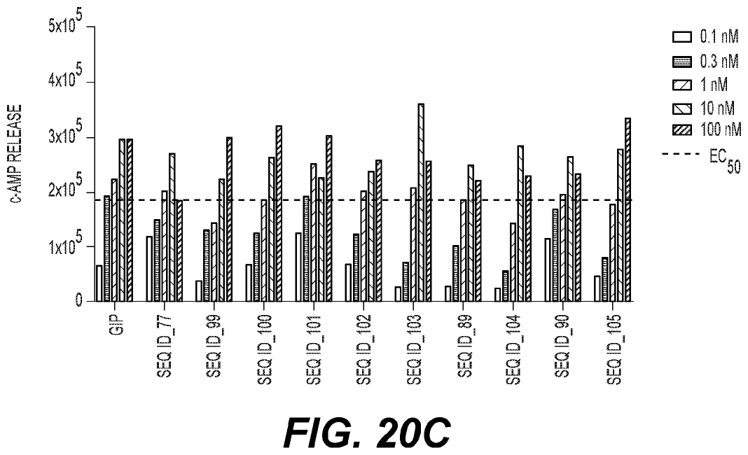

SEQ ID NOs. 99, 100, 101, 102, 103, 104, and 105 were screened using cAMP assay in three target cell lines, GLP-1R+ cells, GCGR+ cells, GIPR+ cells, at five different concentrations compared with SEQ ID NOs. 77, 89, and 90 (FIGS. 20A-20C).

Full screening of SEQ ID NOs. 99, 100, and 101 in cAMP assay were conducted to determine their EC₅₀ the three target cell lines, GLP-1R+ cells, GCGR+ cells, GIPR+ cells compared with SEQ ID NO. 77. The relative ratios of EC₅₀ of their respective native ligand over that of the triple agonist peptides are shown in Table 36.

SEQ ID NOs. 99, 100, and 101 were synthesized with the substitution of 20^{th} amino acid from SEQ ID NO. 77 to improve the stability in the intestinal environment. When compared to SEQ ID NO. 77, in vitro activity of SEQ ID NOs. 99, 100, and 101 was changed according to the amino acid substitution. The activity on GLP-1R was slightly decreased in SEQ ID NO. 99 and the activity of SEQ ID NO. 100 on both GLP-1R and GIPR was decreased compared to SEQ ID NO. 77. In the case of SEQ ID NO. 101, the activity on GLP-1R and GIPR was increased.

TABLE 36
In vitro activities of SEQ ID NOs. 77, 99, 100, and 101.

| SEQ ID | Relative % to native ligand | | | Ratio vs. GLP-1R | | |
|---|---|---|---|---|---|---|
| NO. | GLP-1R | GCGR | GIPR | GLP-1R | GCGR | GIPR |
| 77 | 219 | 56 | 230 | 100 | 26 | 105 |
| 99 | 35 | 28 | 1132 | 100 | 80 | 3237 |
| 100 | 54 | 30 | 55 | 100 | 56 | 103 |
| 101 | 722 | 89 | 472 | 100 | 12 | 65 |

### Example 14: Pharmacokinetic Study in Rats

### Methods

This study was conducted to evaluate the PK profile of SEQ ID NOs. 77, 102, and 103 and compare the PK profile by the different lipidated structure of peptides in rat plasma by IV administration. SEQ ID NOs. 77, 102, and 103 were administrated at 100 nmol/kg, i.v. as shown in Table 37. The peptides were delivered at 0.02% PS80 in PBS and dosing volume was 2.5 mL/kg.

The animals were not fasted prior to dosing. 0.2-0.4 mL of whole blood was taken from vein at each time point (0.167, 0.5, 1, 2, 4, 6, 8, 24, and 48 hr after administration) and transferred into the heparin coated tube.

The resulting plasma of 0.1-0.2 mL obtained by centrifugation of the blood samples was transferred into the tube and stored at −70° C. until analysis. The concentrations of peptides in plasma were determined by LC-MS/MS analysis.

TABLE 37
Treatment dose and regime.

| | | | Dose | |
|---|---|---|---|---|
| Group No. | SEQ ID NO. | N | (nmol/kg) | Route |
| 1 | 77 | 4 | 100 | IV |
| 2 | 102 | 4 | 100 | IV |
| 3 | 103 | 4 | 100 | IV |

### Results

The PK parameters were presented following single IV administration of SEQ ID NOs. 77, 102, and 103 are shown in Table 38.

According to the mean value of PK parameters, SEQ ID NOs. 102 and 103 showed similar PK parameter values although there was a difference in lipid structure, and SEQ ID NO. 77 showed the significantly low PK parameter values in half-life and AUC_{inf}/dose compared with the SEQ ID NOs. 102 and 103.

TABLE 38
Mean PK parameters of SEQ ID NOs. 77, 102, and
103 in rat following single IV administration

| Parameter | SEQ ID NO. 77 | SEQ ID NO. 102 | SEQ ID NO. 103 |
|---|---|---|---|
| Dose (mg/kg) | 0.507 | 0.495 | 0.510 |
| t_{1/2} (hr) | 7.11 ± 0.4 | 9.77 ± 0.89 | 9.59 ± 1.32 |
| | (6%)^{a} | (9%)*^{, b} | (14%)*^{, b} |
| AUC_{inf}/dose | 156.76 ± 27.16 | 672.93 ± 32.13 | 629.51 ± 76.87 |
| (hr · kg/L) | (17%)^{a} | (5%)^{b} | (12%)^{b} |

Each value represents the mean ± SD (CV %) of provided replicates.
*PK parameters calculated with four individual subjects.
Different letters (^{a} and ^{b}) indicate statistically differences (p < 0.05) in the results of PK parameters.

### Example 15: In Vitro Stability Study Against FaSSIF/P and Trypsin

### Methods

This study was conducted to determine the enzymatic stability of SEQ ID NOs. 77, 99, 100, and 101 after substitution of 20th amino acid in artificial intestinal environment.

For the stability against FaSSIF/P, the test samples were prepared by mixing stock solutions of peptide and pancreatin dissolved in FaSSIF solution (pH 6.5) in a weight ratio of 19:1. The test samples were incubated at 37° C. for 120 min. The sampling from test samples was proceeded at the predetermined time points (10, 30, 60, and 120 min) and stopped by 10% TFA solution.

For the stability against trypsin, the test samples were prepared by mixing stock solutions of peptide and trypsin dissolved in 50 mM PBS (pH 7.8) containing 0.02% PS80 in a weight ratio of 10:1. The test samples were incubated at 37° C. for 180 min. The sampling from test samples was proceeded at the predetermined time points (10, 30, 60, 90, 120, and 180 min) and stopped by 10% TFA solution.

Analytical HPLC was performed to determine the remaining amount on an ACQUITY Premier system (Waters, USA) with an ACQUITY CSH C18 column (Waters, USA) at 35° C. using 0.1% TFA in water and acetonitrile. The remaining amount was determined as a percentage of the peak area at the sampling time point relative to the peak area of the initial sample.

### Results

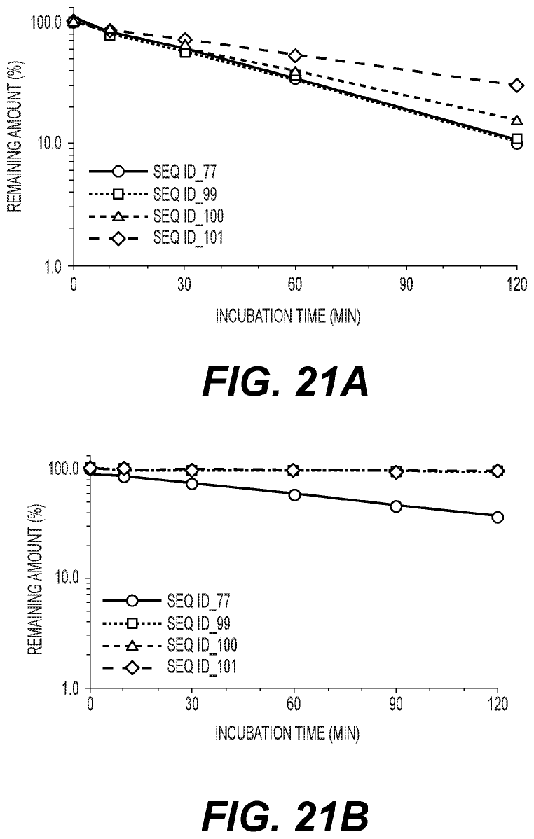

The enzymatic stability of SEQ ID NOs. 99, 100, and 101 was determined in artificial intestinal environment compared with SEQ ID NO. 77. The half-lives determined by remaining amount in FaSSIF/P and trypsin are summarized in Table 39. As a result of this study, SEQ ID NOs. 99 and 100 showed the similar stability against FaSSIF/P and the increased half-lives against trypsin compared with SEQ ID NO. 77. SEQ ID NO. 101 showed the improved stability against both FaSSIF/P and trypsin compared with SEQ ID NO. 77. (FIGS. 21A and 21B).

TABLE 39
In vitro stability study of SEQ ID NOs. 77, 99,
100, and 101 against FaSSIF/P and trypsin.

Half-life (min)

| SEQ ID NO. | FaSSIF/P | Trypsin |
|---|---|---|
| 77 | 36.9 | 92.5 |
| 99 | 37.9 | >433.7 |
| 100 | 45.2 | >433.7 |
| 101 | 70.7 | >433.7 |

### Example 16: In Vivo Efficacy Study in CDA-HFD Mice

### Methods

This study was conducted to evaluate the efficacy of SEQ ID NOs. 77, 99, 100, 101, 102, and 103 to determine the effect of amino acid substitution and lipid structure on efficacy in CDA-HFD mice, the NASH model. SEQ ID NOs. 77, 99, 100, 101, 102, and 103 were administered at 20 nmol/kg, s.c. Q2D for 4 weeks as shown in Table 40. The peptides were delivered at 0.02% PS80 in PBS and dosing volume was 10 mL/kg. Control group was administered by vehicle without peptide.

Body weight changes were monitored Q2D; serum chemistry (end); hepatic TG (end); and histological analysis (end).

TABLE 40
Treatment dose and regime.

| | | | Dose | | Dosing |
|---|---|---|---|---|---|
| Group No. | SEQ ID NO. | N | (nmol/kg) | Route | interval |
| 1 | Vehicle (CHOW) | 5 | — | SC | Q2D |
| 2 | Vehicle (CDA-HFD) | 6 | — | SC | Q2D |
| 3 | 77 | 6 | 20 | SC | Q2D |
| 4 | 99 | 6 | 20 | SC | Q2D |
| 5 | 100 | 6 | 20 | SC | Q2D |
| 6 | 101 | 6 | 20 | SC | Q2D |
| 7 | 102 | 6 | 20 | SC | Q2D |
| 8 | 103 | 6 | 20 | SC | Q2D |

### Results

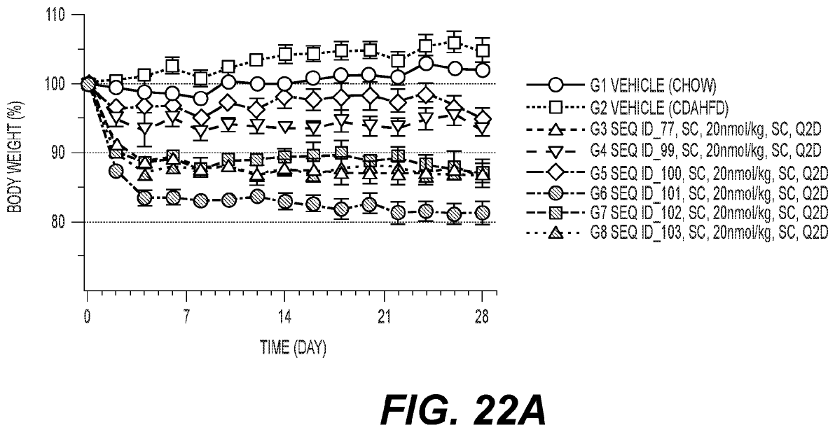

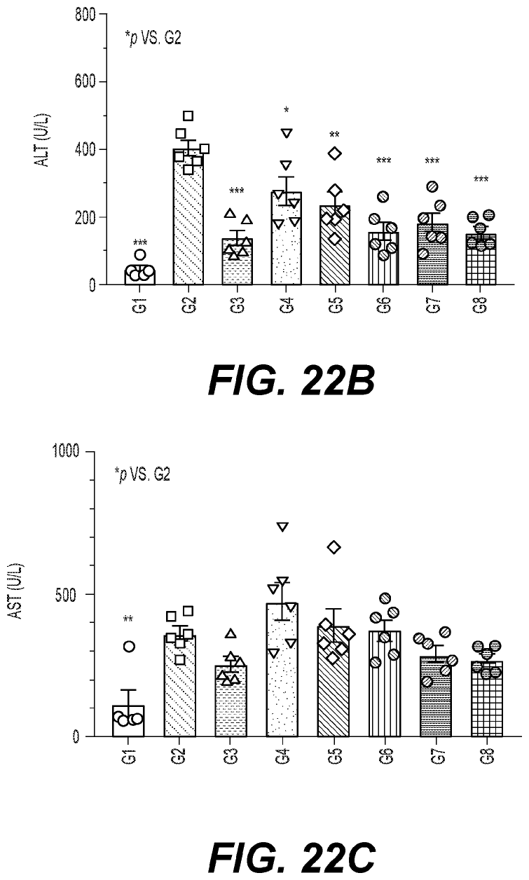

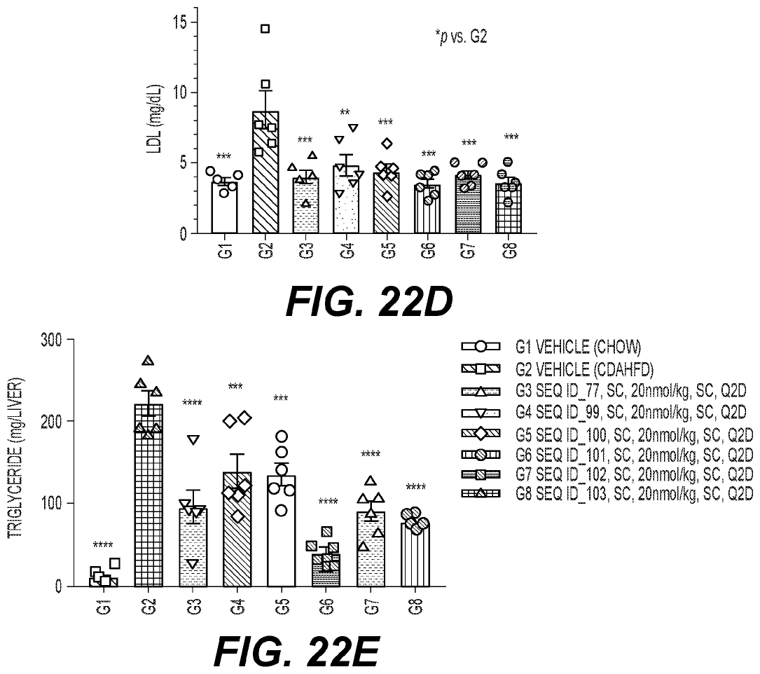

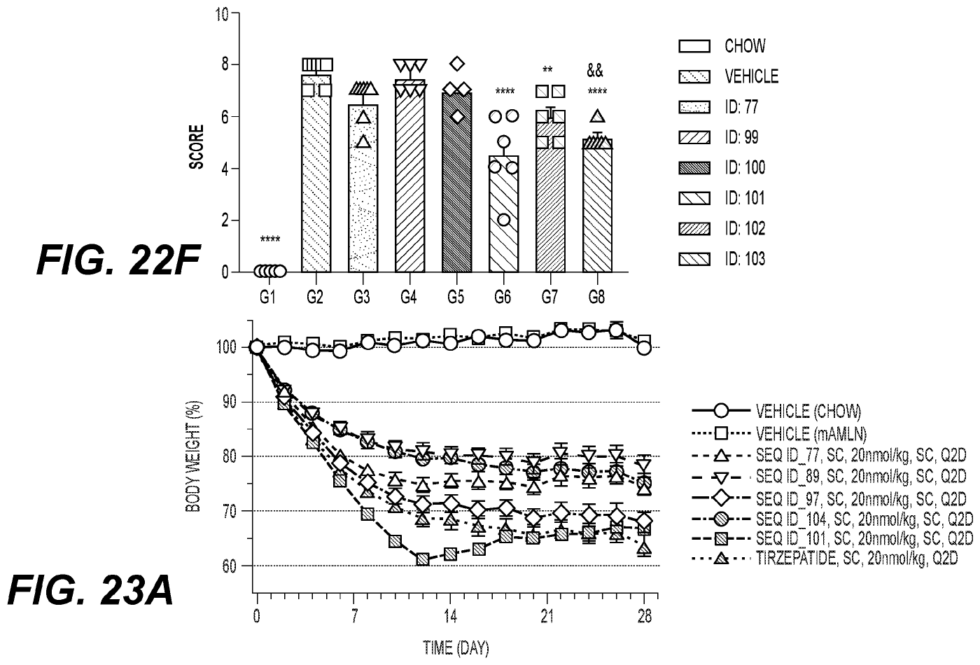

Body weight changes in CDA-HFD mice treated with SEQ ID NOs. 77, 99, 100, 101, 102, and 103 over a period of 4 weeks are shown in FIG. 22A. SEQ ID NOs. 101, 102, and 103 were shown to reduce body weight compared to vehicle-treated control in the NASH mice model. In particular, the greatest weight loss was observed in SEQ ID NO. 101.

At the end of treatment, ALT and AST for hepatocellular, several lipid markers in serum (LDL and TG), histology score were analyzed and shown in FIG. 22B-22F. SEQ ID NOs. 101, 102, and 103 were shown to reduce liver enzymes and liver steatosis significantly compared to vehicle-treated control. The hepatic steatosis score was significantly reduced in SEQ ID NOs. 101 and 103 compared to CDA-HFD control. The scores of hepatocytes inflammation and NAS score were statistically reduced in SEQ ID NOs. 101, 102, and 103. SEQ ID NO. 101 was the most decreased the NAS score.

Among the tested groups, SEQ ID NO. 101 had the most excellent efficacy in the NASH model. The lipid structure of SEQ ID NO. 103 had the superior efficacy compared to SEQ ID NO. 77.

### Example 17: In Vivo Efficacy Study in mAMLN Mice

### Methods

This study was conducted to evaluate the efficacy of SEQ ID NOs. 77, 89, 97, 104, and 101 for conformation the effects of biotinylation, amino acid substitution and changed lipid structure on efficacy in mAMLN mice, the obese and NASH model. Tirzepatide, GLP-1 and GIP dual agonist from Eli Lilly, was used as a positive control. SEQ ID NOs. 77, 89, 97, 104, 101, and Tirzepatide were administered at 20 nmol/kg, s.c. Q2D for 4 weeks as shown in Table 41. The peptides were delivered at 0.02% PS80 in PBS and dosing volume was 10 mL/kg. Control groups were administered by the solution without peptide.

Body weight changes were monitored Q2D; liver weight (end); visceral fat weight (end); serum chemistry (end); hepatic TG (end); inflammation markers (end); and histological analysis (end).

TABLE 41
Treatment dose and regime.

| | | | Dose | | Dosing |
|---|---|---|---|---|---|
| Group No. | SEQ ID NO. | N | (nmol/kg) | Route | interval |
| 1 | Vehicle (CHOW) | 5 | — | SC | Q2D |
| 2 | Vehicle (CDA-HFD) | 6 | — | SC | Q2D |
| 3 | 77 | 6 | 20 | SC | Q2D |
| 4 | 89 | 6 | 20 | SC | Q2D |
| 5 | 97 | 6 | 20 | SC | Q2D |
| 6 | 104 | 6 | 20 | SC | Q2D |
| 7 | 101 | 6 | 20 | SC | Q2D |
| 8 | Tirzepatide | 6 | 20 | SC | Q2D |

### Results

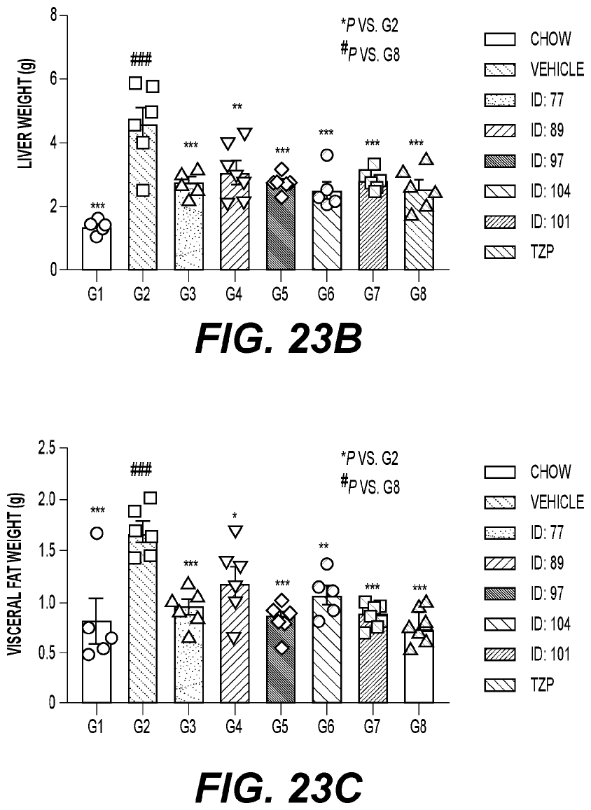

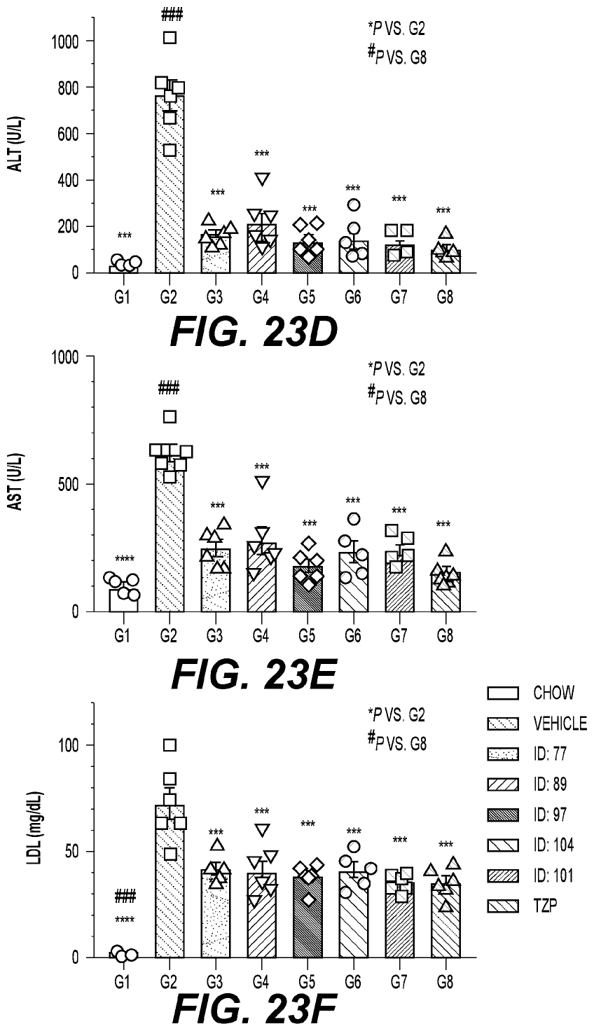

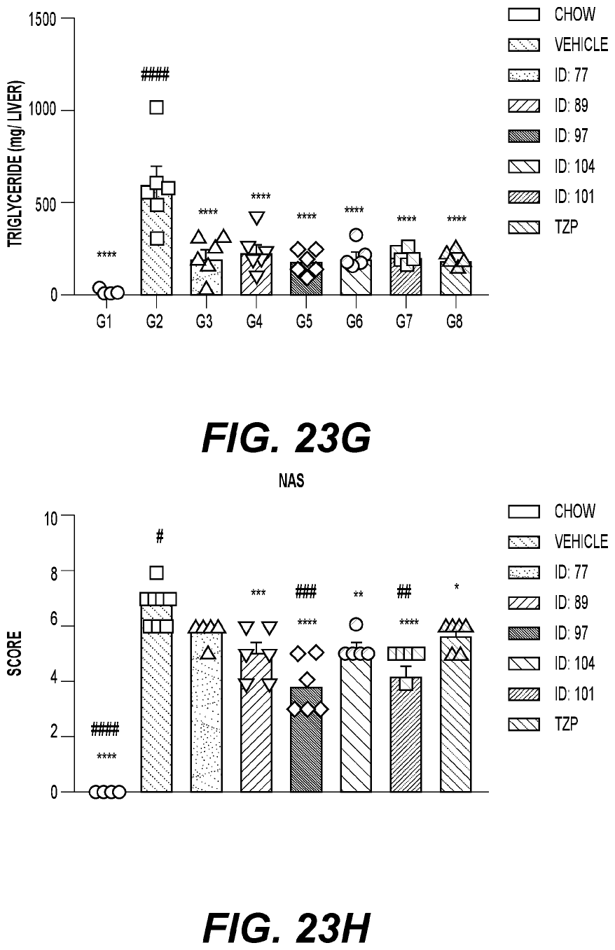

Body weight changes, liver weight, visceral fat weight, ALT, AST, LDL, hepatic TG and histological scores in mAMLN mice treated with SEQ ID NOs. 77, 87, 97, 104, and 101 as well as tirzepatide as positive control over a period of 4 weeks are shown in FIG. 23A-23H.

After 4 weeks of administration, SEQ ID NOs. 97 and 101 as well as tirzepatide showed similar body weight loss to the CHOW control group. Additionally, SEQ ID NOs. 97 and 101 significantly reduced liver weight, visceral fat weight, liver enzymes, and liver steatosis compared to vehicle-treated control, and most of these parameters reduced to similar level as the positive control, tirzepatide.

In the histological score, hepatocytes ballooning was significantly decreased in all treated groups and hepatic steatosis was significantly reduced in SEQ ID NOs. 89, 97, 104, and 101 treated group. Overall, the NAS score in SEQ ID NOs. 97 and 101 were statistically decreased more than the level of tirzepatide.

### Example 18: Peptide Screening for Optimization of Sequence

### Methods

Based on the results of previous studies, SEQ ID NOs. 66 and 101 were selected for further optimization of peptide sequence. Peptides derived from SEQ ID NOs. 66 and 101 were synthesized by substituting the 20th amino acid with αR to improve the intestinal stability. Sequences are in Table 42 with lipidation at indicated lysine residue (K* or K**), biotinylation at indicated lysine residue (B*) and α-methyl-arginine (αR). The fatty acid derivatives used for conjugation here are 2OEG-γGlu-C18 (K*) or 2OEG-γGlu-C20 (K**). The biotin derivative used for conjugation here is biotin monomer.

In vitro activity study was performed to determine the optimal sequence by conforming the activity for each receptor. The peptides were tested in vitro for activity using cAMP assays on GLP-1R, GCGR, and GIPR as previously described. Their signals from cAMP production were compared to native ligand peptide (GLP-1, GCG or GIP).

TABLE 42
Peptide sequence

| SEQ ID | |
|---|---|
| NO. | Amino Acid Sequence |
| 106 | YAibQGT FTSDY SK*YLD YMMQαR DFVQW LLEGG |
| | PSSGA PPPS |
| 107 | Y AibQGT FTSDY SK**YLD YMMQαR DFVQW |
| | LLEGG PSSGA PPPS |
| 108 | YAibQGT FTSDY SK**LLD YMMQαR DFVQW |
| | LLEGG PSSGA PPPS |
| 109 | Y AibQGT FTSDY SK*YLD YMMQαR DFVB*W |
| | LLEGG PSSGA PPPS |
| 110 | YAibQGT FTSDY SK**YLD YMMQαR DFVB*W |
| | LLEGG PSSGA PPPS |
| 111 | YAibQGT FTSDY SK*LLD YMMQαR DFVB*W LLEGG |
| | PSSGA PPPS |
| 112 | Y AibQGT FTSDY SK**LLD YMMQαR DFVB*W |
| | LLEGG PSSGA PPPS |

### Results

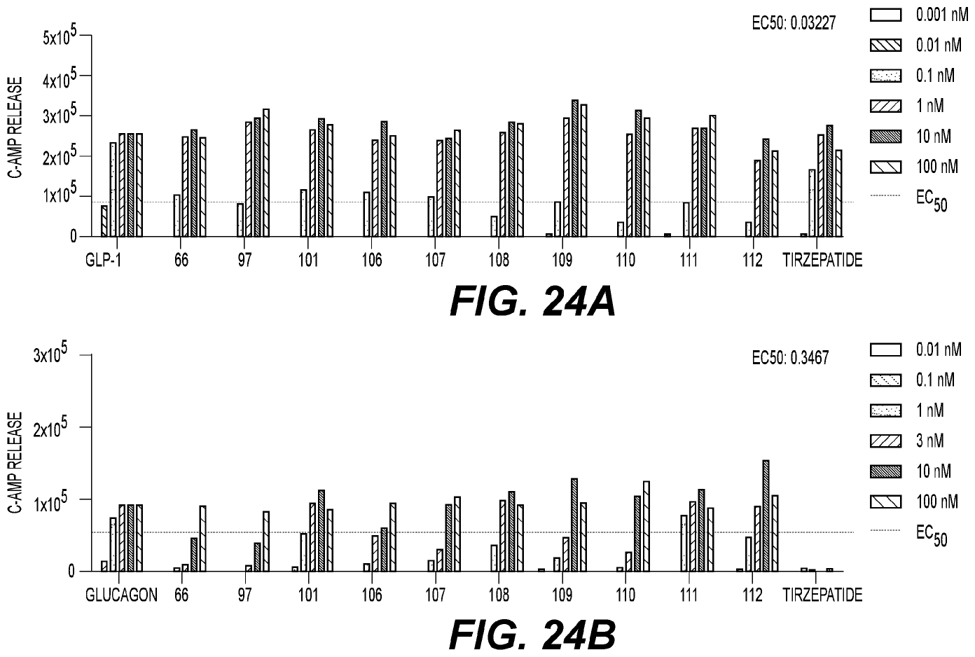

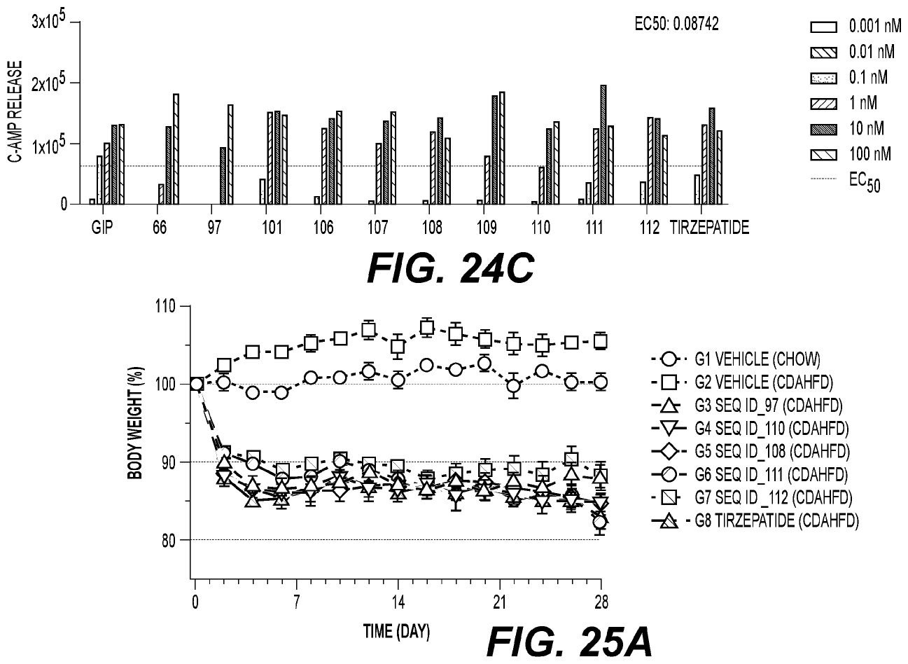

SEQ ID NOs. 106, 107, 108, 109, 110, 111, and 112 were screened using cAMP assay in three target cell lines, GLP-1R+ cells, GCGR+ cells, GIPR+ cells, at six different concentrations compared with SEQ ID NOs. 66, 97, and 101 as well as tirzepatide (FIGS. 24A-24C).

Full screening of SEQ ID NOs. 107, 108, 110, and 112 in cAMP assay were conducted to determine their EC₅₀ the three target cell lines, GLP-1R+ cells, GCGR+ cells, GIPR+ cells. The relative ratios of EC₅₀ of their respective native ligand over that of the triple agonist peptides are shown in Table 43.

TABLE 43
In vitro activities of SEQ ID NOs. 107, 108, 110, and 112.

| SEQ ID | Relative % to native ligand | | | Ratio vs. GLP-1R | | |
|---|---|---|---|---|---|---|
| NO. | GLP-1R | GCGR | GIPR | GLP-1R | GCGR | GIPR |
| 107 | 37 | 14 | 200 | 100 | 37 | 537 |
| 108 | 46 | 39 | 451 | 100 | 83 | 971 |
| 110 | 39 | 11 | 105 | 100 | 29 | 272 |
| 112 | 39 | 20 | 279 | 100 | 51 | 715 |

### Example 19: In Vivo Efficacy Study in CDA-HFD Mice

### Methods

This study was conducted to evaluate the efficacy of SEQ ID NOs. 97, 110, 108, 111 and 112 to determine the effect of lipid structure and biotinylation on efficacy in CDA-HFD treated mice. SEQ ID NOs. 97, 110, 108, 111 and 112 were administered at 20 nmol/kg, s.c. Q2D for 4 weeks compared to tirzepatide as shown in Table 44. The peptides were delivered at 0.02% PS80 in PBS and dosing volume was 10 mL/kg. Control groups were administered by vehicle without peptide.

Body weight changes were monitored Q2D; liver weight (end); serum chemistry (end) and hepatic TG (end).

TABLE 44
Treatment dose and regime.

| | | | Dose | | Dosing |
|---|---|---|---|---|---|
| Group No. | SEQ ID NO. | N | (nmol/kg) | Route | interval |
| 1 | Vehicle (CHOW) | 5 | — | SC | Q2D |
| 2 | Vehicle (CDA-HFD) | 6 | — | SC | Q2D |
| 3 | 97 | 6 | 20 | SC | Q2D |
| 4 | 110 | 6 | 20 | SC | Q2D |
| 5 | 108 | 6 | 20 | SC | Q2D |
| 6 | 111 | 6 | 20 | SC | Q2D |
| 7 | 112 | 6 | 20 | SC | Q2D |
| 8 | Tirzepatide | 6 | 20 | SC | Q2D |

### Results

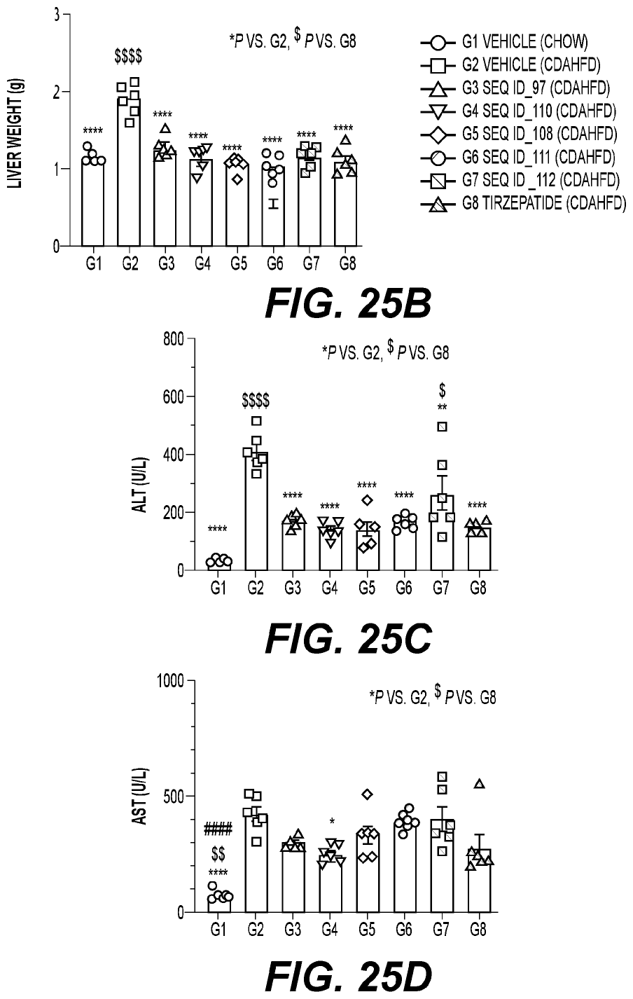

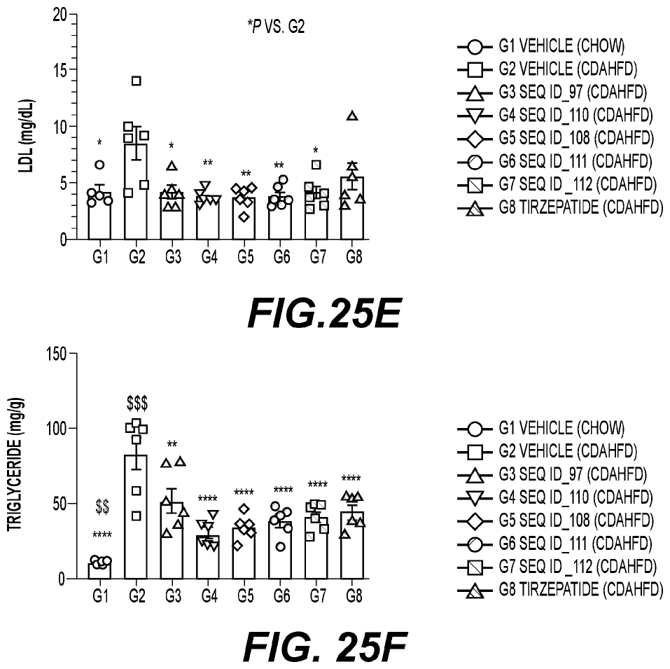

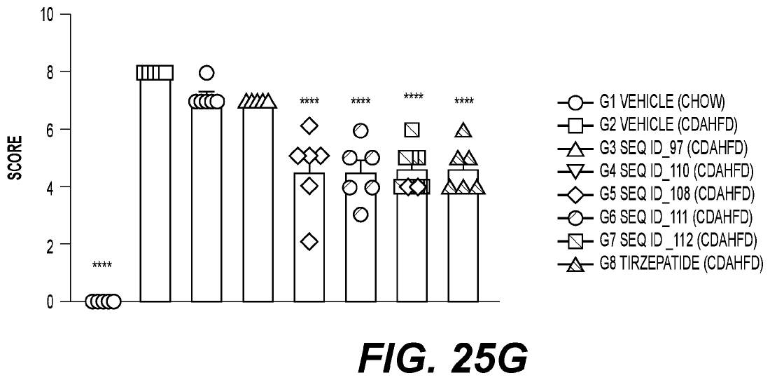

Body weight changes, liver weight, ALT, AST, LDL, and hepatic TG in CDA-HFD mice treated with NOs. 97, 108, 110, 111 and 112 as well as tirzepatide as positive control over a period of 4 weeks are shown in FIG. 25A-25G.

After 4 weeks of administration, all treated peptides and tirzepatide showed similar body weight loss, liver weight loss and liver-related markers improvement.

### Example 20: Intestinal Absorption Following Enteral Administration in Rats

### Methods

This study was conducted to evaluate the intestinal absorption of peptides following enteral administration of oral pharmaceutical formulations. The pharmaceutical formulation was administered in a form of suspensions by intraduodenal (ID) injection in rats. SEQ ID_87, 89 and 90 was accurately weighed and dissolved in 10 mM PBS containing polysorbate 80 (pH 7.4, vehicle I). The mixture was vortexed for 5 min to get a clear solution. Then, 2.5 mL of vehicle was added to the mixture and vortexed for 30 min to get homogenous opaque suspension. The composition of Vehicle II was sodium chenodeoxycholate, and propyl gallate in polysorbate 80 in PBS. The animals were not fasted prior to dosing. 0.2˜ 0.4 mL of whole blood was taken from vein at each time point (0, 0.167, 0.5, 1, 2, 4, 8, 24, and 48 hrs after IV or ID administration). The collected whole blood was incubated at room temperature for 20 min and tubes were centrifugated at 13,000 rpm for 10 min at 4° C. The resulting serum 0.1˜ 0.2 mL was transferred into the tube and stored at −70° C. until analysis. The concentrations of peptides in serum were determined by LC-MS/MS analysis. PK parameters were analyzed by non-compartmental analysis using Phoenix WinNonlin 5.0.1. software (Pharsight Corporation, Mountain View, CA, USA).

TABLE 45
Treatment dose and regime.

| | | | Dose | | Dosing |
|---|---|---|---|---|---|
| Group No. | SEQ ID NO. | N | (nmol/kg) | Route | interval |
| 1 | SEQ ID_87 | 4 | 300 | intraduodenal | Single |
| 2 | SEQ ID_89 | 5 | 300 | intraduodenal | Single |
| 3 | SEQ ID_90 | 5 | 300 | intraduodenal | Single |

### Results

The PK parameters were presented following single intraduodenal administration of SEQ ID NOs. 87, 89 and 90 are shown in Table 46. Intestinal absorption was confirmed after enteral administration of the oral pharmaceutical formulations.

According to the mean value of PK parameters, Following the ID administration of SEQ ID_87, 89, and 90 in rats, the mean Cₘₐₓ values were 289.53, 331.73, and 281.58 ng/ml, while Cₘₐₓ was reached at 0.25, 0.43 and 0.30 hr, respectively. The t1/2 were 6.17, 7.24, and 8.37 and AUCinf values were 1,861.06, 2,793.64, and 2,488.01 ng·hr/mL after ID administration for SEQ ID_87, 89, and 90, respectively.

TABLE 46
Mean PK parameters of SEQ ID NOs. 87, 89, and 90 following
single intraduodenal administration in rats.

| PK parameters | SEQ ID_87 | SEQ ID_89 | SEQ ID_90 |
|---|---|---|---|
| Group | 1 | 2 | 3 |
| Replicates (n) | 4 | 5 | 5 |
| Dose Route | intraduodenal | intraduodenal | intraduodenal |
| Cₘₐₓ (ng/mL) | 289.53 ± 109.9 | 331.73 ± 124.32 | 281.58 ± 88.52 |
| Tₘₐₓ (hr) | 0.25 ± 0.17 | 0.43 ± 0.15 | 0.30 ± 0.18 |
| t_{1/2} (hr) | 6.17 ± 1.36 | 7.24 ± 2.05 | 8.37 ± 0.92 |
| MRT_{inf} (hr) | 8.19 ± 1.92 | 9.89 ± 3.91 | 11.84 ± 1.48 |
| AUC_{inf} | 1,861.06 ± 1,056.95 | 2,793.64 ± 1,464.66 | 2,488.01 ± 824.48 |
| (ng · hr/mL) | | | |

Each value represents the mean ± SD of provided replicates.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize or be able to ascertain using no more than routine experimentation, many equivalents to the specific embodiments of the invention described herein. Such equivalents are intended to be encompassed by the following claims.

1. A triple agonist peptide or analog thereof having activities at each of a glucagon-like peptide-1 (GLP-1) receptor, a glucagon receptor, and a glucose-dependent insulinotropic polypeptide (GIP) receptor, comprising
(SEQ ID NO: 131)
X₁X₂X₃GTFTSDX₁₀SX₁₂X₁₃LDX₁₆X₁₇X₁₈X₁₉X₂₀X₂₁X₂₂X₂₃X₂₄X₂₅X₂₆X₂₇
X₂₈GX₃₀X₃₁SX₃₃X₃₄X₃₅PP X₃₈ X₃₉X₄₀,
wherein X₁ is H or Y;
X₂ is A or 2-aminoisobutyric acid (Aib);
X₃ is Q or E;
X₁₂ is R, W or K;
X₁₃ is L or Y;
X₁₉ is Q, A or T;
X₂₁ is D or L;
X₂₂ is F or R;
X₂₃ is V, G or D;
X₂₅ is W, Y or A;
X₂₆ is L or D;
X₂₇ is I, L, M, G or P;
X₃₀ is G or P;
X₃₁ is P or S;
X₃₃ is S or G;
X₃₄ is G or A;
X₃₅ is A or P;
X₃₈ is P or S;
X₃₉ is absent, S, or C;
X₄₀ is absent, or C;
with an optional amide modification of the C-terminus and
X₁₀, X₁₆, X₁₇, X₁₈, X₂₀, X₂₄, and X₂₈, any one of the 20 amino acids except cysteine, alternatively, X₁₇, X₁₈, X₂₀ are non-natural amino acids, independently selected from the group consisting of methoxinine, 2-aminoisobutyric acid, and alpha-methyl-arginine.

2. The triple agonist peptide or analog of claim 1, wherein
X₁₀ is Y, W, K, F, H, S, L, A, E, M, Q, or D;
X₁₆ is Y, Q, G, K, S, R, F, P, or A;
X₁₇ is M, Y, Q, K, S, W, P, D, A, F, or methoxinine;
X₁₈ is A, I, M, W, T, D, Y, or methoxinine;
X₂₀ is R, Q, H, G, A, P, N, K, Aib, or alpha-methyl-arginine;
X₂₄ is Q, D, K, L, N, W, or M; and
X₂₈ is N, E, G, D, H, or Q.

3. The triple agonist peptide or analog of claim 1, having amino acid sequence of any one of SEQ ID NOs: 1-130.

4. The triple agonist peptide or analog of claim 1, wherein the peptide or analog is conjugated to one or more of biotin moieties, fatty acids, or polyethylene glycols, and derivatives thereof, optionally via one or more spacers.

5. The triple agonist peptide or analog of claim 4, wherein the one or more biotin moieties, fatty acids, or polyethylene glycols, and derivatives thereof, are conjugated to the amino acid sequence of any one of SEQ ID NOs: 1-130 via one or more amino acid residues selected from the group consisting of cysteine and lysine.

6. The triple agonist peptide or analog of claim 5, wherein one or more amino acid residues of cysteine and lysine are introduced to the amino acid sequence of any one of SEQ ID NOs: 1-130 by substitution or insertion to allow conjugation to the one or more biotin moieties, fatty acids, or polyethylene glycols, and derivatives thereof.

7. The triple agonist peptide or analog of claim 3, wherein one or more amino acid residues of lysine at position 10, lysine at position 12, lysine at position 17, lysine at position 20, lysine at position 24, one or more C-terminal cysteine residues are introduced to the amino acid sequence of any one of SEQ ID NOs: 1-130 by substitution or insertion to allow conjugation to the one or more biotin moieties, fatty acids, or polyethylene glycols, and derivatives thereof.

8. The triple agonist peptide or analog of claim 4, wherein the biotin moieties and derivatives thereof suitable for conjugation are selected from the group consisting of Biotin N-hydroxysuccinimide ester, N-Biotinoyl-N′-(6-maleimidohexanoyl)hydrazide, 3-Maleimidopropionate-Lys(Biotin)-Lys(Biotin)-CONH₂, 3-Maleimidopropionate-Lys(Biotin)-Lys(Biotin)-Lys(Biotin)-CONH₂, propionate-N-hydroxysuccinimide ester-PEG-Lys(Biotin)-Lys(Biotin)-Lys(Biotin)-CONH₂ and 3-Maleimidopropionate-PEG-Lys(Biotin)-Lys(Biotin)-Lys(Biotin)-CONH₂.

9. The triple agonist peptide or analog of claim 4, wherein the fatty acids and derivatives thereof suitable for conjugation are C16-C22 fatty acids via one or more hydrophilic spacers.

10. The triple agonist peptide or analog of claim 9, wherein the hydrophilic spacers are γGlu or 8-amino-3,6-dioxaoctanoic acid.

11. The triple agonist peptide or analog of claim 4, wherein the fatty acids or derivatives thereof suitable for conjugation are selected from the group consisting of C16-NHS, C16-MAL, C18-NHS, C18-MAL, C16-γGlu-NHS, C16-γGlu-MAL, C18-γGlu-NHS, C18-γGlu-MAL, C18-γGlu-NHS, C18-γGlu-OEG-MAL, C18-γGlu-2OEG-NHS, C18-γGlu-2OEG-MAL, C20-γGlu-2OEG-NHS, C20-γGlu-2OEG-MAL, C18-γGlu-2OEG-TFP, C18-γGlu-2OEG-NPC, and C20-γGlu-2OEG-NPC.

12. A pharmaceutical formulation comprising the triple agonist peptide or analog of claim 1.

13. A method of treating one or more diseases selected from the group consisting of obesity, diabetes, and non-alcoholic fatty liver disease in a subject in need thereof, comprising
administering to an effective amount of the pharmaceutical formulation of claim 12 to treat or alleviate one or more symptom of the one or more diseases.

14. The method of claim 13, wherein the pharmaceutical formulation is administered in an amount effective to induce weight loss, reduce the body fat, reduce food intake, improve glucose homeostasis, or combinations thereof, in a normal or obese patient.

15. The method of claim 13, wherein the subject is suffering from non-alcoholic fatty liver disease.

16. The method of claim 15, wherein the non-alcoholic fatty liver disease is one or more diseases selected from the group consisting of non-alcoholic fatty liver, non-alcoholic steatohepatitis, liver cirrhosis, and liver cancer.

17. The method of claim 15, wherein the pharmaceutical formulation is administered in an amount effective to inhibit or reduce serum levels of one or more of alanine aminotransferase, aspartate aminotransferase, triglyceride, gamma-glutamyl transferase, total cholesterol, low density lipoprotein, fasting blood sugar or combinations thereof.

18. The method of claim 15, wherein the pharmaceutical formulation is administered in an amount effective to reduce one or more of steatosis, inflammation, ballooning, fibrosis, cirrhosis, or combinations thereof.

19. The method of claim 13, wherein the pharmaceutical formulation is administered via a route selected from the group consisting of enteral administration and parenteral administration.

20. The method of claim 13, wherein the pharmaceutical formulation is administered via oral administration or subcutaneous administration.

21. The method of claim 13, wherein the pharmaceutical formulation is administered in a form selected from the group consisting of pills, capsules, tablets, liquids, and suspensions.

22. The method of claim 13, wherein the pharmaceutical formulation is administered at an interval selected from the group consisting of once a month, once every two weeks, once a week, once every three days, once every two days, once daily, or twice daily.

23. The method of claim 13, wherein the pharmaceutical formulation is administered the subject once a week for up to 6 months.

24. The method of claim 13, wherein the pharmaceutical formulation is administered to the subject for a duration of between one and 10 days, weeks, or months, inclusive.

25. The method of claim 13, wherein the pharmaceutical formulation is administered to a human subject at a dose of between 0.001 mg/kg body weight of the subject and 10 mg/kg body weight of the subject, inclusive.

26. The method of claim 13, wherein the pharmaceutical formulation is administered to a human subject at a dose of between 0.01 mg/kg body weight of the subject and 1 mg/kg body weight of the subject, inclusive.

27. The method of claim 13, wherein the pharmaceutical formulation is administered to a human subject at a dose of between 1.0 mg and 100 mg, inclusive.
